SEQUENCE LISTING (577 sequences)

SEQ ID NO: 1 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 2 - RNA, 25 nt, synthetic construct
  misc_feature 1..25: note = oligonucleotide
  source 1..25: mol_type = other RNA
gtacctccaacatcaaggaagatgg

SEQ ID NO: 3 - RNA, 25 nt, synthetic construct
  misc_feature 1..25: note = oligonucleotide
  source 1..25: mol_type = other RNA
tacctccaacatcaaggaagatggc

SEQ ID NO: 4 - RNA, 25 nt, synthetic construct
  misc_feature 1..25: note = oligonucleotide
  source 1..25: mol_type = other RNA
acctccaacatcaaggaagatggca

SEQ ID NO: 5 - RNA, 25 nt, synthetic construct
  misc_feature 1..25: note = oligonucleotide
  source 1..25: mol_type = other RNA
cctccaacatcaaggaagatggcat

SEQ ID NO: 6 - RNA, 25 nt, synthetic construct
  misc_feature 1..25: note = oligonucleotide
  source 1..25: mol_type = other RNA
ctccaacatcaaggaagatggcatt

SEQ ID NO: 7 - RNA, 25 nt, synthetic construct
  misc_feature 1..25: note = oligonucleotide
  source 1..25: mol_type = other RNA
tccaacatcaaggaagatggcattt

SEQ ID NO: 8 - RNA, 25 nt, synthetic construct
  misc_feature 1..25: note = oligonucleotide
  source 1..25: mol_type = other RNA
ccaacatcaaggaagatggcatttc

SEQ ID NO: 9 - RNA, 25 nt, synthetic construct
  misc_feature 1..25: note = oligonucleotide
  source 1..25: mol_type = other RNA
caacatcaaggaagatggcatttct

SEQ ID NO: 10 - RNA, 25 nt, synthetic construct
  misc_feature 1..25: note = oligonucleotide
  source 1..25: mol_type = other RNA
aacatcaaggaagatggcatttcta

SEQ ID NO: 11 - RNA, 25 nt, synthetic construct
  misc_feature 1..25: note = oligonucleotide
  source 1..25: mol_type = other RNA
acatcaaggaagatggcatttctag

SEQ ID NO: 12 - RNA, 25 nt, synthetic construct
  misc_feature 1..25: note = oligonucleotide
  source 1..25: mol_type = other RNA
catcaaggaagatggcatttctagt

SEQ ID NO: 13 - RNA, 25 nt, synthetic construct
  misc_feature 1..25: note = oligonucleotide
  source 1..25: mol_type = other RNA
atcaaggaagatggcatttctagtt

SEQ ID NO: 14 - RNA, 25 nt, synthetic construct
  misc_feature 1..25: note = oligonucleotide
  source 1..25: mol_type = other RNA
tcaaggaagatggcatttctagttt

SEQ ID NO: 15 - RNA, 25 nt, synthetic construct
  misc_feature 1..25: note = oligonucleotide
  source 1..25: mol_type = other RNA
caaggaagatggcatttctagtttg

SEQ ID NO: 16 - RNA, 25 nt, synthetic construct
  misc_feature 1..25: note = oligonucleotide
  source 1..25: mol_type = other RNA
aaggaagatggcatttctagtttgg

SEQ ID NO: 17 - RNA, 25 nt, synthetic construct
  misc_feature 1..25: note = oligonucleotide
  source 1..25: mol_type = other RNA
aggaagatggcatttctagtttgga

SEQ ID NO: 18 - RNA, 25 nt, synthetic construct
  misc_feature 1..25: note = oligonucleotide
  source 1..25: mol_type = other RNA
ggaagatggcatttctagtttggag

SEQ ID NO: 19 - RNA, 25 nt, synthetic construct
  misc_feature 1..25: note = oligonucleotide
  source 1..25: mol_type = other RNA
gaagatggcatttctagtttggaga

SEQ ID NO: 20 - RNA, 25 nt, synthetic construct
  misc_feature 1..25: note = oligonucleotide
  source 1..25: mol_type = other RNA
aagatggcatttctagtttggagat

SEQ ID NO: 21 - RNA, 25 nt, synthetic construct
  misc_feature 1..25: note = oligonucleotide
  source 1..25: mol_type = other RNA
agatggcatttctagtttggagatg

SEQ ID NO: 22 - RNA, 25 nt, synthetic construct
  misc_feature 1..25: note = oligonucleotide
  source 1..25: mol_type = other RNA
gatggcatttctagtttggagatgg

SEQ ID NO: 23 - RNA, 25 nt, synthetic construct
  misc_feature 1..25: note = oligonucleotide
  source 1..25: mol_type = other RNA
atggcatttctagtttggagatggc

SEQ ID NO: 24 - RNA, 25 nt, synthetic construct
  misc_feature 1..25: note = oligonucleotide
  source 1..25: mol_type = other RNA
tggcatttctagtttggagatggca

SEQ ID NO: 25 - RNA, 25 nt, synthetic construct
  misc_feature 1..25: note = oligonucleotide
  source 1..25: mol_type = other RNA
ggcatttctagtttggagatggcag

SEQ ID NO: 26 - RNA, 25 nt, synthetic construct
  misc_feature 1..25: note = oligonucleotide
  source 1..25: mol_type = other RNA
gcatttctagtttggagatggcagt

SEQ ID NO: 27 - RNA, 25 nt, synthetic construct
  misc_feature 1..25: note = oligonucleotide
  source 1..25: mol_type = other RNA
catttctagtttggagatggcagtt

SEQ ID NO: 28 - RNA, 25 nt, synthetic construct
  misc_feature 1..25: note = oligonucleotide
  source 1..25: mol_type = other RNA
atttctagtttggagatggcagttt

SEQ ID NO: 29 - RNA, 25 nt, synthetic construct
  misc_feature 1..25: note = oligonucleotide
  source 1..25: mol_type = other RNA
tttctagtttggagatggcagtttc

SEQ ID NO: 30 - RNA, 25 nt, synthetic construct
  misc_feature 1..25: note = oligonucleotide
  source 1..25: mol_type = other RNA
ttctagtttggagatggcagtttcc

SEQ ID NO: 31 - RNA, 25 nt, synthetic construct
  misc_feature 1..25: note = oligonucleotide
  source 1..25: mol_type = other RNA
tctagtttggagatggcagtttcct

SEQ ID NO: 32 - RNA, 25 nt, synthetic construct
  misc_feature 1..25: note = oligonucleotide
  source 1..25: mol_type = other RNA
ctagtttggagatggcagtttcctt

SEQ ID NO: 33 - RNA, 25 nt, synthetic construct
  misc_feature 1..25: note = oligonucleotide
  source 1..25: mol_type = other RNA
tagtttggagatggcagtttcctta

SEQ ID NO: 34 - RNA, 25 nt, synthetic construct
  misc_feature 1..25: note = oligonucleotide
  source 1..25: mol_type = other RNA
agtttggagatggcagtttccttag

SEQ ID NO: 35 - RNA, 25 nt, synthetic construct
  misc_feature 1..25: note = oligonucleotide
  source 1..25: mol_type = other RNA
gtttggagatggcagtttccttagt

SEQ ID NO: 36 - RNA, 25 nt, synthetic construct
  misc_feature 1..25: note = oligonucleotide
  source 1..25: mol_type = other RNA
tttggagatggcagtttccttagta

SEQ ID NO: 37 - RNA, 25 nt, synthetic construct
  misc_feature 1..25: note = oligonucleotide
  source 1..25: mol_type = other RNA
ttggagatggcagtttccttagtaa

SEQ ID NO: 38 - RNA, 25 nt, synthetic construct
  misc_feature 1..25: note = oligonucleotide
  source 1..25: mol_type = other RNA
tggagatggcagtttccttagtaac

SEQ ID NO: 39 - RNA, 25 nt, synthetic construct
  misc_feature 1..25: note = oligonucleotide
  source 1..25: mol_type = other RNA
gagatggcagtttccttagtaacca

SEQ ID NO: 40 - RNA, 25 nt, synthetic construct
  misc_feature 1..25: note = oligonucleotide
  source 1..25: mol_type = other RNA
agatggcagtttccttagtaaccac

SEQ ID NO: 41 - RNA, 25 nt, synthetic construct
  misc_feature 1..25: note = oligonucleotide
  source 1..25: mol_type = other RNA
gatggcagtttccttagtaaccaca

SEQ ID NO: 42 - RNA, 25 nt, synthetic construct
  misc_feature 1..25: note = oligonucleotide
  source 1..25: mol_type = other RNA
atggcagtttccttagtaaccacag

SEQ ID NO: 43 - RNA, 25 nt, synthetic construct
  misc_feature 1..25: note = oligonucleotide
  source 1..25: mol_type = other RNA
tggcagtttccttagtaaccacagg

SEQ ID NO: 44 - RNA, 25 nt, synthetic construct
  misc_feature 1..25: note = oligonucleotide
  source 1..25: mol_type = other RNA
ggcagtttccttagtaaccacaggt

SEQ ID NO: 45 - RNA, 25 nt, synthetic construct
  misc_feature 1..25: note = oligonucleotide
  source 1..25: mol_type = other RNA
gcagtttccttagtaaccacaggtt

SEQ ID NO: 46 - RNA, 25 nt, synthetic construct
  misc_feature 1..25: note = oligonucleotide
  source 1..25: mol_type = other RNA
cagtttccttagtaaccacaggttg

SEQ ID NO: 47 - RNA, 25 nt, synthetic construct
  misc_feature 1..25: note = oligonucleotide
  source 1..25: mol_type = other RNA
agtttccttagtaaccacaggttgt

SEQ ID NO: 48 - RNA, 25 nt, synthetic construct
  misc_feature 1..25: note = oligonucleotide
  source 1..25: mol_type = other RNA
gtttccttagtaaccacaggttgtg

SEQ ID NO: 49 - RNA, 25 nt, synthetic construct
  misc_feature 1..25: note = oligonucleotide
  source 1..25: mol_type = other RNA
tttccttagtaaccacaggttgtgt

SEQ ID NO: 50 - RNA, 25 nt, synthetic construct
  misc_feature 1..25: note = oligonucleotide
  source 1..25: mol_type = other RNA
ttccttagtaaccacaggttgtgtc

SEQ ID NO: 51 - RNA, 25 nt, synthetic construct
  misc_feature 1..25: note = oligonucleotide
  source 1..25: mol_type = other RNA
tccttagtaaccacaggttgtgtca

SEQ ID NO: 52 - RNA, 25 nt, synthetic construct
  misc_feature 1..25: note = oligonucleotide
  source 1..25: mol_type = other RNA
ccttagtaaccacaggttgtgtcac

SEQ ID NO: 53 - RNA, 25 nt, synthetic construct
  misc_feature 1..25: note = oligonucleotide
  source 1..25: mol_type = other RNA
cttagtaaccacaggttgtgtcacc

SEQ ID NO: 54 - RNA, 25 nt, synthetic construct
  misc_feature 1..25: note = oligonucleotide
  source 1..25: mol_type = other RNA
ttagtaaccacaggttgtgtcacca

SEQ ID NO: 55 - RNA, 25 nt, synthetic construct
  misc_feature 1..25: note = oligonucleotide
  source 1..25: mol_type = other RNA
tagtaaccacaggttgtgtcaccag

SEQ ID NO: 56 - RNA, 25 nt, synthetic construct
  misc_feature 1..25: note = oligonucleotide
  source 1..25: mol_type = other RNA
agtaaccacaggttgtgtcaccaga

SEQ ID NO: 57 - RNA, 25 nt, synthetic construct
  misc_feature 1..25: note = oligonucleotide
  source 1..25: mol_type = other RNA
gtaaccacaggttgtgtcaccagag

SEQ ID NO: 58 - RNA, 25 nt, synthetic construct
  misc_feature 1..25: note = oligonucleotide
  source 1..25: mol_type = other RNA
taaccacaggttgtgtcaccagagt

SEQ ID NO: 59 - RNA, 25 nt, synthetic construct
  misc_feature 1..25: note = oligonucleotide
  source 1..25: mol_type = other RNA
aaccacaggttgtgtcaccagagta

SEQ ID NO: 60 - RNA, 25 nt, synthetic construct
  misc_feature 1..25: note = oligonucleotide
  source 1..25: mol_type = other RNA
accacaggttgtgtcaccagagtaa

SEQ ID NO: 61 - RNA, 25 nt, synthetic construct
  misc_feature 1..25: note = oligonucleotide
  source 1..25: mol_type = other RNA
ccacaggttgtgtcaccagagtaac

SEQ ID NO: 62 - RNA, 25 nt, synthetic construct
  misc_feature 1..25: note = oligonucleotide
  source 1..25: mol_type = other RNA
cacaggttgtgtcaccagagtaaca

SEQ ID NO: 63 - RNA, 25 nt, synthetic construct
  misc_feature 1..25: note = oligonucleotide
  source 1..25: mol_type = other RNA
acaggttgtgtcaccagagtaacag

SEQ ID NO: 64 - RNA, 25 nt, synthetic construct
  misc_feature 1..25: note = oligonucleotide
  source 1..25: mol_type = other RNA
caggttgtgtcaccagagtaacagt

SEQ ID NO: 65 - RNA, 25 nt, synthetic construct
  misc_feature 1..25: note = oligonucleotide
  source 1..25: mol_type = other RNA
aggttgtgtcaccagagtaacagtc

SEQ ID NO: 66 - RNA, 25 nt, synthetic construct
  misc_feature 1..25: note = oligonucleotide
  source 1..25: mol_type = other RNA
ggttgtgtcaccagagtaacagtct

SEQ ID NO: 67 - RNA, 25 nt, synthetic construct
  misc_feature 1..25: note = oligonucleotide
  source 1..25: mol_type = other RNA
gttgtgtcaccagagtaacagtctg

SEQ ID NO: 68 - RNA, 25 nt, synthetic construct
  misc_feature 1..25: note = oligonucleotide
  source 1..25: mol_type = other RNA
ttgtgtcaccagagtaacagtctga

SEQ ID NO: 69 - RNA, 25 nt, synthetic construct
  misc_feature 1..25: note = oligonucleotide
  source 1..25: mol_type = other RNA
tgtgtcaccagagtaacagtctgag

SEQ ID NO: 70 - RNA, 25 nt, synthetic construct
  misc_feature 1..25: note = oligonucleotide
  source 1..25: mol_type = other RNA
gtgtcaccagagtaacagtctgagt

SEQ ID NO: 71 - RNA, 25 nt, synthetic construct
  misc_feature 1..25: note = oligonucleotide
  source 1..25: mol_type = other RNA
tgtcaccagagtaacagtctgagta

SEQ ID NO: 72 - RNA, 25 nt, synthetic construct
  misc_feature 1..25: note = oligonucleotide
  source 1..25: mol_type = other RNA
gtcaccagagtaacagtctgagtag

SEQ ID NO: 73 - RNA, 25 nt, synthetic construct
  misc_feature 1..25: note = oligonucleotide
  source 1..25: mol_type = other RNA
tcaccagagtaacagtctgagtagg

SEQ ID NO: 74 - RNA, 25 nt, synthetic construct
  misc_feature 1..25: note = oligonucleotide
  source 1..25: mol_type = other RNA
caccagagtaacagtctgagtagga

SEQ ID NO: 75 - RNA, 25 nt, synthetic construct
  misc_feature 1..25: note = oligonucleotide
  source 1..25: mol_type = other RNA
accagagtaacagtctgagtaggag

SEQ ID NO: 76 - RNA, 25 nt, synthetic construct
  misc_feature 1..25: note = oligonucleotide
  source 1..25: mol_type = other RNA
tttgccgctgcccaatgccatcctg

SEQ ID NO: 77 - RNA, 25 nt, synthetic construct
  misc_feature 1..25: note = oligonucleotide
  source 1..25: mol_type = other RNA
attcaatgttctgacaacagtttgc

SEQ ID NO: 78 - RNA, 25 nt, synthetic construct
  misc_feature 1..25: note = oligonucleotide
  source 1..25: mol_type = other RNA
ccagttgcattcaatgttctgacaa

SEQ ID NO: 79 - RNA, 22 nt, synthetic construct
  misc_feature 1..22: note = oligonucleotide
  source 1..22: mol_type = other RNA
cagttgcattcaatgttctgac

SEQ ID NO: 80 - RNA, 20 nt, synthetic construct
  misc_feature 1..20: note = oligonucleotide
  source 1..20: mol_type = other RNA
agttgcattcaatgttctga

SEQ ID NO: 81 - RNA, 21 nt, synthetic construct
  misc_feature 1..21: note = oligonucleotide
  source 1..21: mol_type = other RNA
gattgctgaattatttcttcc

SEQ ID NO: 82 - RNA, 25 nt, synthetic construct
  misc_feature 1..25: note = oligonucleotide
  source 1..25: mol_type = other RNA
gattgctgaattatttcttccccag

SEQ ID NO: 83 - RNA, 25 nt, synthetic construct
  misc_feature 1..25: note = oligonucleotide
  source 1..25: mol_type = other RNA
attgctgaattatttcttccccagt

SEQ ID NO: 84 - RNA, 25 nt, synthetic construct
  misc_feature 1..25: note = oligonucleotide
  source 1..25: mol_type = other RNA
ttgctgaattatttcttccccagtt

SEQ ID NO: 85 - RNA, 25 nt, synthetic construct
  misc_feature 1..25: note = oligonucleotide
  source 1..25: mol_type = other RNA
tgctgaattatttcttccccagttg

SEQ ID NO: 86 - RNA, 25 nt, synthetic construct
  misc_feature 1..25: note = oligonucleotide
  source 1..25: mol_type = other RNA
gctgaattatttcttccccagttgc

SEQ ID NO: 87 - RNA, 25 nt, synthetic construct
  misc_feature 1..25: note = oligonucleotide
  source 1..25: mol_type = other RNA
ctgaattatttcttccccagttgca

SEQ ID NO: 88 - RNA, 25 nt, synthetic construct
  misc_feature 1..25: note = oligonucleotide
  source 1..25: mol_type = other RNA
tgaattatttcttccccagttgcat

SEQ ID NO: 89 - RNA, 25 nt, synthetic construct
  misc_feature 1..25: note = oligonucleotide
  source 1..25: mol_type = other RNA
gaattatttcttccccagttgcatt

SEQ ID NO: 90 - RNA, 25 nt, synthetic construct
  misc_feature 1..25: note = oligonucleotide
  source 1..25: mol_type = other RNA
aattatttcttccccagttgcattc

SEQ ID NO: 91 - RNA, 25 nt, synthetic construct
  misc_feature 1..25: note = oligonucleotide
  source 1..25: mol_type = other RNA
attatttcttccccagttgcattca

SEQ ID NO: 92 - RNA, 25 nt, synthetic construct
  misc_feature 1..25: note = oligonucleotide
  source 1..25: mol_type = other RNA
ttatttcttccccagttgcattcaa

SEQ ID NO: 93 - RNA, 25 nt, synthetic construct
  misc_feature 1..25: note = oligonucleotide
  source 1..25: mol_type = other RNA
tatttcttccccagttgcattcaat

SEQ ID NO: 94 - RNA, 25 nt, synthetic construct
  misc_feature 1..25: note = oligonucleotide
  source 1..25: mol_type = other RNA
atttcttccccagttgcattcaatg

SEQ ID NO: 95 - RNA, 25 nt, synthetic construct
  misc_feature 1..25: note = oligonucleotide
  source 1..25: mol_type = other RNA
tttcttccccagttgcattcaatgt

SEQ ID NO: 96 - RNA, 25 nt, synthetic construct
  misc_feature 1..25: note = oligonucleotide
  source 1..25: mol_type = other RNA
ttcttccccagttgcattcaatgtt

SEQ ID NO: 97 - RNA, 25 nt, synthetic construct
  misc_feature 1..25: note = oligonucleotide
  source 1..25: mol_type = other RNA
tcttccccagttgcattcaatgttc

SEQ ID NO: 98 - RNA, 25 nt, synthetic construct
  misc_feature 1..25: note = oligonucleotide
  source 1..25: mol_type = other RNA
cttccccagttgcattcaatgttct

SEQ ID NO: 99 - RNA, 25 nt, synthetic construct
  misc_feature 1..25: note = oligonucleotide
  source 1..25: mol_type = other RNA
ttccccagttgcattcaatgttctg

SEQ ID NO: 100 - RNA, 25 nt, synthetic construct
  misc_feature 1..25: note = oligonucleotide
  source 1..25: mol_type = other RNA
tccccagttgcattcaatgttctga

SEQ ID NO: 101 - RNA, 25 nt, synthetic construct
  misc_feature 1..25: note = oligonucleotide
  source 1..25: mol_type = other RNA
ccccagttgcattcaatgttctgac

SEQ ID NO: 102 - RNA, 25 nt, synthetic construct
  misc_feature 1..25: note = oligonucleotide
  source 1..25: mol_type = other RNA
cccagttgcattcaatgttctgaca

SEQ ID NO: 103 - RNA, 25 nt, synthetic construct
  misc_feature 1..25: note = oligonucleotide
  source 1..25: mol_type = other RNA
ccagttgcattcaatgttctgacaa

SEQ ID NO: 104 - RNA, 25 nt, synthetic construct
  misc_feature 1..25: note = oligonucleotide
  source 1..25: mol_type = other RNA
cagttgcattcaatgttctgacaac

SEQ ID NO: 105 - RNA, 25 nt, synthetic construct
  misc_feature 1..25: note = oligonucleotide
  source 1..25: mol_type = other RNA
agttgcattcaatgttctgacaaca

SEQ ID NO: 106 - RNA, 20 nt, synthetic construct
  misc_feature 1..20: note = oligonucleotide
  source 1..20: mol_type = other RNA
tcctgtagaatactggcatc

SEQ ID NO: 107 - RNA, 27 nt, synthetic construct
  misc_feature 1..27: note = oligonucleotide
  source 1..27: mol_type = other RNA
tgcagacctcctgccaccgcagattca

SEQ ID NO: 108 - RNA, 34 nt, synthetic construct
  misc_feature 1..34: note = oligonucleotide
  source 1..34: mol_type = other RNA
ttgcagacctcctgccaccgcagattcaggcttc

SEQ ID NO: 109 - RNA, 25 nt, synthetic construct
  misc_feature 1..25: note = oligonucleotide
  source 1..25: mol_type = other RNA
gttgcattcaatgttctgacaacag

SEQ ID NO: 110 - RNA, 25 nt, synthetic construct
  misc_feature 1..25: note = oligonucleotide
  source 1..25: mol_type = other RNA
ttgcattcaatgttctgacaacagt

SEQ ID NO: 111 - RNA, 25 nt, synthetic construct
  misc_feature 1..25: note = oligonucleotide
  source 1..25: mol_type = other RNA
tgcattcaatgttctgacaacagtt

SEQ ID NO: 112 - RNA, 25 nt, synthetic construct
  misc_feature 1..25: note = oligonucleotide
  source 1..25: mol_type = other RNA
gcattcaatgttctgacaacagttt

SEQ ID NO: 113 - RNA, 25 nt, synthetic construct
  misc_feature 1..25: note = oligonucleotide
  source 1..25: mol_type = other RNA
cattcaatgttctgacaacagtttg

SEQ ID NO: 114 - RNA, 25 nt, synthetic construct
  misc_feature 1..25: note = oligonucleotide
  source 1..25: mol_type = other RNA
attcaatgttctgacaacagtttgc

SEQ ID NO: 115 - RNA, 25 nt, synthetic construct
  misc_feature 1..25: note = oligonucleotide
  source 1..25: mol_type = other RNA
tcaatgttctgacaacagtttgccg

SEQ ID NO: 116 - RNA, 25 nt, synthetic construct
  misc_feature 1..25: note = oligonucleotide
  source 1..25: mol_type = other RNA
caatgttctgacaacagtttgccgc

SEQ ID NO: 117 - RNA, 25 nt, synthetic construct
  misc_feature 1..25: note = oligonucleotide
  source 1..25: mol_type = other RNA
aatgttctgacaacagtttgccgct

SEQ ID NO: 118 - RNA, 25 nt, synthetic construct
  misc_feature 1..25: note = oligonucleotide
  source 1..25: mol_type = other RNA
atgttctgacaacagtttgccgctg

SEQ ID NO: 119 - RNA, 25 nt, synthetic construct
  misc_feature 1..25: note = oligonucleotide
  source 1..25: mol_type = other RNA
tgttctgacaacagtttgccgctgc

SEQ ID NO: 120 - RNA, 25 nt, synthetic construct
  misc_feature 1..25: note = oligonucleotide
  source 1..25: mol_type = other RNA
gttctgacaacagtttgccgctgcc

SEQ ID NO: 121 - RNA, 25 nt, synthetic construct
  misc_feature 1..25: note = oligonucleotide
  source 1..25: mol_type = other RNA
ttctgacaacagtttgccgctgccc

SEQ ID NO: 122 - RNA, 25 nt, synthetic construct
  misc_feature 1..25: note = oligonucleotide
  source 1..25: mol_type = other RNA
tctgacaacagtttgccgctgccca

SEQ ID NO: 123 - RNA, 25 nt, synthetic construct
  misc_feature 1..25: note = oligonucleotide
  source 1..25: mol_type = other RNA
ctgacaacagtttgccgctgcccaa

SEQ ID NO: 124 - RNA, 25 nt, synthetic construct
  misc_feature 1..25: note = oligonucleotide
  source 1..25: mol_type = other RNA
tgacaacagtttgccgctgcccaat

SEQ ID NO: 125 - RNA, 25 nt, synthetic construct
  misc_feature 1..25: note = oligonucleotide
  source 1..25: mol_type = other RNA
gacaacagtttgccgctgcccaatg

SEQ ID NO: 126 - RNA, 25 nt, synthetic construct
  misc_feature 1..25: note = oligonucleotide
  source 1..25: mol_type = other RNA
acaacagtttgccgctgcccaatgc

SEQ ID NO: 127 - RNA, 25 nt, synthetic construct
  misc_feature 1..25: note = oligonucleotide
  source 1..25: mol_type = other RNA
caacagtttgccgctgcccaatgcc

SEQ ID NO: 128 - RNA, 25 nt, synthetic construct
  misc_feature 1..25: note = oligonucleotide
  source 1..25: mol_type = other RNA
aacagtttgccgctgcccaatgcca

SEQ ID NO: 129 - RNA, 25 nt, synthetic construct
  misc_feature 1..25: note = oligonucleotide
  source 1..25: mol_type = other RNA
acagtttgccgctgcccaatgccat

SEQ ID NO: 130 - RNA, 25 nt, synthetic construct
  misc_feature 1..25: note = oligonucleotide
  source 1..25: mol_type = other RNA
cagtttgccgctgcccaatgccatc

SEQ ID NO: 131 - RNA, 25 nt, synthetic construct
  misc_feature 1..25: note = oligonucleotide
  source 1..25: mol_type = other RNA
agtttgccgctgcccaatgccatcc

SEQ ID NO: 132 - RNA, 25 nt, synthetic construct
  misc_feature 1..25: note = oligonucleotide
  source 1..25: mol_type = other RNA
gtttgccgctgcccaatgccatcct

SEQ ID NO: 133 - RNA, 25 nt, synthetic construct
  misc_feature 1..25: note = oligonucleotide
  source 1..25: mol_type = other RNA
tttgccgctgcccaatgccatcctg

SEQ ID NO: 134 - RNA, 25 nt, synthetic construct
  misc_feature 1..25: note = oligonucleotide
  source 1..25: mol_type = other RNA
ttgccgctgcccaatgccatcctgg

SEQ ID NO: 135 - RNA, 25 nt, synthetic construct
  misc_feature 1..25: note = oligonucleotide
  source 1..25: mol_type = other RNA
tgccgctgcccaatgccatcctgga

SEQ ID NO: 136 - RNA, 25 nt, synthetic construct
  misc_feature 1..25: note = oligonucleotide
  source 1..25: mol_type = other RNA
gccgctgcccaatgccatcctggag

SEQ ID NO: 137 - RNA, 25 nt, synthetic construct
  misc_feature 1..25: note = oligonucleotide
  source 1..25: mol_type = other RNA
ccgctgcccaatgccatcctggagt

SEQ ID NO: 138 - RNA, 25 nt, synthetic construct
  misc_feature 1..25: note = oligonucleotide
  source 1..25: mol_type = other RNA
cgctgcccaatgccatcctggagtt

SEQ ID NO: 139 - RNA, 20 nt, synthetic construct
  misc_feature 1..20: note = oligonucleotide
  source 1..20: mol_type = other RNA
tgtttttgaggattgctgaa

SEQ ID NO: 140 - RNA, 40 nt, synthetic construct
  misc_feature 1..40: note = oligonucleotide
  source 1..40: mol_type = other RNA
tgttctgacaacagtttgccgctgcccaatgccatcctgg

SEQ ID NO: 141 - RNA, 25 nt, synthetic construct
  misc_feature 1..25: note = oligonucleotide
  source 1..25: mol_type = other RNA
ctctggcctgtcctaagacctgctc

SEQ ID NO: 142 - RNA, 25 nt, synthetic construct
  misc_feature 1..25: note = oligonucleotide
  source 1..25: mol_type = other RNA
tctggcctgtcctaagacctgctca

SEQ ID NO: 143 - RNA, 25 nt, synthetic construct
  misc_feature 1..25: note = oligonucleotide
  source 1..25: mol_type = other RNA
ctggcctgtcctaagacctgctcag

SEQ ID NO: 144 - RNA, 25 nt, synthetic construct
  misc_feature 1..25: note = oligonucleotide
  source 1..25: mol_type = other RNA
tggcctgtcctaagacctgctcagc

SEQ ID NO: 145 - RNA, 25 nt, synthetic construct
  misc_feature 1..25: note = oligonucleotide
  source 1..25: mol_type = other RNA
ggcctgtcctaagacctgctcagct

SEQ ID NO: 146 - RNA, 25 nt, synthetic construct
  misc_feature 1..25: note = oligonucleotide
  source 1..25: mol_type = other RNA
gcctgtcctaagacctgctcagctt

SEQ ID NO: 147 - RNA, 25 nt, synthetic construct
  misc_feature 1..25: note = oligonucleotide
  source 1..25: mol_type = other RNA
cctgtcctaagacctgctcagcttc

SEQ ID NO: 148 - RNA, 25 nt, synthetic construct
  misc_feature 1..25: note = oligonucleotide
  source 1..25: mol_type = other RNA
ctgtcctaagacctgctcagcttct

SEQ ID NO: 149 - RNA, 25 nt, synthetic construct
  misc_feature 1..25: note = oligonucleotide
  source 1..25: mol_type = other RNA
tgtcctaagacctgctcagcttctt

SEQ ID NO: 150 - RNA, 25 nt, synthetic construct
  misc_feature 1..25: note = oligonucleotide
  source 1..25: mol_type = other RNA
gtcctaagacctgctcagcttcttc

SEQ ID NO: 151 - RNA, 25 nt, synthetic construct
  misc_feature 1..25: note = oligonucleotide
  source 1..25: mol_type = other RNA
tcctaagacctgctcagcttcttcc

SEQ ID NO: 152 - RNA, 25 nt, synthetic construct
  misc_feature 1..25: note = oligonucleotide
  source 1..25: mol_type = other RNA
cctaagacctgctcagcttcttcct

SEQ ID NO: 153 - RNA, 25 nt, synthetic construct
  misc_feature 1..25: note = oligonucleotide
  source 1..25: mol_type = other RNA
ctaagacctgctcagcttcttcctt

SEQ ID NO: 154 - RNA, 25 nt, synthetic construct
  misc_feature 1..25: note = oligonucleotide
  source 1..25: mol_type = other RNA
taagacctgctcagcttcttcctta

SEQ ID NO: 155 - RNA, 25 nt, synthetic construct
  misc_feature 1..25: note = oligonucleotide
  source 1..25: mol_type = other RNA
aagacctgctcagcttcttccttag

SEQ ID NO: 156 - RNA, 25 nt, synthetic construct
  misc_feature 1..25: note = oligonucleotide
  source 1..25: mol_type = other RNA
agacctgctcagcttcttccttagc

SEQ ID NO: 157 - RNA, 25 nt, synthetic construct
  misc_feature 1..25: note = oligonucleotide
  source 1..25: mol_type = other RNA
gacctgctcagcttcttccttagct

SEQ ID NO: 158 - RNA, 25 nt, synthetic construct
  misc_feature 1..25: note = oligonucleotide
  source 1..25: mol_type = other RNA
acctgctcagcttcttccttagctt

SEQ ID NO: 159 - RNA, 25 nt, synthetic construct
  misc_feature 1..25: note = oligonucleotide
  source 1..25: mol_type = other RNA
cctgctcagcttcttccttagcttc

SEQ ID NO: 160 - RNA, 25 nt, synthetic construct
  misc_feature 1..25: note = oligonucleotide
  source 1..25: mol_type = other RNA
ctgctcagcttcttccttagcttcc

SEQ ID NO: 161 - RNA, 25 nt, synthetic construct
  misc_feature 1..25: note = oligonucleotide
  source 1..25: mol_type = other RNA
tgctcagcttcttccttagcttcca

SEQ ID NO: 162 - RNA, 25 nt, synthetic construct
  misc_feature 1..25: note = oligonucleotide
  source 1..25: mol_type = other RNA
gctcagcttcttccttagcttccag

SEQ ID NO: 163 - RNA, 25 nt, synthetic construct
  misc_feature 1..25: note = oligonucleotide
  source 1..25: mol_type = other RNA
ctcagcttcttccttagcttccagc

SEQ ID NO: 164 - RNA, 25 nt, synthetic construct
  misc_feature 1..25: note = oligonucleotide
  source 1..25: mol_type = other RNA
tcagcttcttccttagcttccagcc

SEQ ID NO: 165 - RNA, 25 nt, synthetic construct
  misc_feature 1..25: note = oligonucleotide
  source 1..25: mol_type = other RNA
cagcttcttccttagcttccagcca

SEQ ID NO: 166 - RNA, 25 nt, synthetic construct
  misc_feature 1..25: note = oligonucleotide
  source 1..25: mol_type = other RNA
agcttcttccttagcttccagccat

SEQ ID NO: 167 - RNA, 25 nt, synthetic construct
  misc_feature 1..25: note = oligonucleotide
  source 1..25: mol_type = other RNA
gcttcttccttagcttccagccatt

SEQ ID NO: 168 - RNA, 25 nt, synthetic construct
  misc_feature 1..25: note = oligonucleotide
  source 1..25: mol_type = other RNA
cttcttccttagcttccagccattg

SEQ ID NO: 169 - RNA, 25 nt, synthetic construct
  misc_feature 1..25: note = oligonucleotide
  source 1..25: mol_type = other RNA
ttcttccttagcttccagccattgt

SEQ ID NO: 170 - RNA, 25 nt, synthetic construct
  misc_feature 1..25: note = oligonucleotide
  source 1..25: mol_type = other RNA
tcttccttagcttccagccattgtg

SEQ ID NO: 171 - RNA, 25 nt, synthetic construct
  misc_feature 1..25: note = oligonucleotide
  source 1..25: mol_type = other RNA
cttccttagcttccagccattgtgt

SEQ ID NO: 172 - RNA, 25 nt, synthetic construct
  misc_feature 1..25: note = oligonucleotide
  source 1..25: mol_type = other RNA
ttccttagcttccagccattgtgtt

SEQ ID NO: 173 - RNA, 25 nt, synthetic construct
  misc_feature 1..25: note = oligonucleotide
  source 1..25: mol_type = other RNA
tccttagcttccagccattgtgttg

SEQ ID NO: 174 - RNA, 25 nt, synthetic construct
  misc_feature 1..25: note = oligonucleotide
  source 1..25: mol_type = other RNA
ccttagcttccagccattgtgttga

SEQ ID NO: 175 - RNA, 25 nt, synthetic construct
  misc_feature 1..25: note = oligonucleotide
  source 1..25: mol_type = other RNA
cttagcttccagccattgtgttgaa

SEQ ID NO: 176 - RNA, 25 nt, synthetic construct
  misc_feature 1..25: note = oligonucleotide
  source 1..25: mol_type = other RNA
ttagcttccagccattgtgttgaat

SEQ ID NO: 177 - RNA, 25 nt, synthetic construct
  misc_feature 1..25: note = oligonucleotide
  source 1..25: mol_type = other RNA
tagcttccagccattgtgttgaatc

SEQ ID NO: 178 - RNA, 25 nt, synthetic construct
  misc_feature 1..25: note = oligonucleotide
  source 1..25: mol_type = other RNA
agcttccagccattgtgttgaatcc

SEQ ID NO: 179 - RNA, 25 nt, synthetic construct
  misc_feature 1..25: note = oligonucleotide
  source 1..25: mol_type = other RNA
gcttccagccattgtgttgaatcct

SEQ ID NO: 180 - RNA, 25 nt, synthetic construct
  misc_feature 1..25: note = oligonucleotide
  source 1..25: mol_type = other RNA
cttccagccattgtgttgaatcctt

SEQ ID NO: 181 - RNA, 25 nt, synthetic construct
  misc_feature 1..25: note = oligonucleotide
  source 1..25: mol_type = other RNA
ttccagccattgtgttgaatccttt

SEQ ID NO: 182 - RNA, 25 nt, synthetic construct
  misc_feature 1..25: note = oligonucleotide
  source 1..25: mol_type = other RNA
tccagccattgtgttgaatccttta

SEQ ID NO: 183 - RNA, 25 nt, synthetic construct
  misc_feature 1..25: note = oligonucleotide
  source 1..25: mol_type = other RNA
ccagccattgtgttgaatcctttaa

SEQ ID NO: 184 - RNA, 25 nt, synthetic construct
  misc_feature 1..25: note = oligonucleotide
  source 1..25: mol_type = other RNA
cagccattgtgttgaatcctttaac

SEQ ID NO: 185 - RNA, 25 nt, synthetic construct
  misc_feature 1..25: note = oligonucleotide
  source 1..25: mol_type = other RNA
agccattgtgttgaatcctttaaca

SEQ ID NO: 186 - RNA, 25 nt, synthetic construct
  misc_feature 1..25: note = oligonucleotide
  source 1..25: mol_type = other RNA
gccattgtgttgaatcctttaacat

SEQ ID NO: 187 - RNA, 25 nt, synthetic construct
  misc_feature 1..25: note = oligonucleotide
  source 1..25: mol_type = other RNA
ccattgtgttgaatcctttaacatt

SEQ ID NO: 188 - RNA, 25 nt, synthetic construct
  misc_feature 1..25: note = oligonucleotide
  source 1..25: mol_type = other RNA
cattgtgttgaatcctttaacattt

SEQ ID NO: 189 - RNA, 20 nt, synthetic construct
  misc_feature 1..20: note = oligonucleotide
  source 1..20: mol_type = other RNA
tcagcttctgttagccactg

SEQ ID NO: 190 - RNA, 20 nt, synthetic construct
  misc_feature 1..20: note = oligonucleotide
  source 1..20: mol_type = other RNA
ttcagcttctgttagccact

SEQ ID NO: 191 - RNA, 21 nt, synthetic construct
  misc_feature 1..21: note = oligonucleotide
  source 1..21: mol_type = other RNA
ttcagcttctgttagccactg

SEQ ID NO: 192 - RNA, 21 nt, synthetic construct
  misc_feature 1..21: note = oligonucleotide
  source 1..21: mol_type = other RNA
tcagcttctgttagccactga

SEQ ID NO: 193 - RNA, 22 nt, synthetic construct
  misc_feature 1..22: note = oligonucleotide
  source 1..22: mol_type = other RNA
ttcagcttctgttagccactga

SEQ ID NO: 194 - RNA, 21 nt, synthetic construct
  misc_feature 1..21: note = oligonucleotide
  source 1..21: mol_type = other RNA
tcagcttctgttagccactga

SEQ ID NO: 195 - RNA, 22 nt, synthetic construct
  misc_feature 1..22: note = oligonucleotide
  source 1..22: mol_type = other RNA
ttcagcttctgttagccactga

SEQ ID NO: 196 - RNA, 22 nt, synthetic construct
  misc_feature 1..22: note = oligonucleotide
  source 1..22: mol_type = other RNA
tcagcttctgttagccactgat

SEQ ID NO: 197 - RNA, 23 nt, synthetic construct
  misc_feature 1..23: note = oligonucleotide
  source 1..23: mol_type = other RNA
ttcagcttctgttagccactgat

SEQ ID NO: 198 - RNA, 23 nt, synthetic construct
  misc_feature 1..23: note = oligonucleotide
  source 1..23: mol_type = other RNA
tcagcttctgttagccactgatt

SEQ ID NO: 199 - RNA, 24 nt, synthetic construct
  misc_feature 1..24: note = oligonucleotide
  source 1..24: mol_type = other RNA
ttcagcttctgttagccactgatt

SEQ ID NO: 200 - RNA, 24 nt, synthetic construct
  misc_feature 1..24: note = oligonucleotide
  source 1..24: mol_type = other RNA
tcagcttctgttagccactgatta

SEQ ID NO: 201 - RNA, 24 nt, synthetic construct
  misc_feature 1..24: note = oligonucleotide
  source 1..24: mol_type = other RNA
ttcagcttctgttagccactgata

SEQ ID NO: 202 - RNA, 25 nt, synthetic construct
  misc_feature 1..25: note = oligonucleotide
  source 1..25: mol_type = other RNA
tcagcttctgttagccactgattaa

SEQ ID NO: 203 - RNA, 26 nt, synthetic construct
  misc_feature 1..26: note = oligonucleotide
  source 1..26: mol_type = other RNA
ttcagcttctgttagccactgattaa

SEQ ID NO: 204 - RNA, 26 nt, synthetic construct
  misc_feature 1..26: note = oligonucleotide
  source 1..26: mol_type = other RNA
tcagcttctgttagccactgattaaa

SEQ ID NO: 205 - RNA, 27 nt, synthetic construct
  misc_feature 1..27: note = oligonucleotide
  source 1..27: mol_type = other RNA
ttcagcttctgttagccactgattaaa

SEQ ID NO: 206 - RNA, 19 nt, synthetic construct
  misc_feature 1..19: note = oligonucleotide
  source 1..19: mol_type = other RNA
cagcttctgttagccactg

SEQ ID NO: 207 - RNA, 21 nt, synthetic construct
  misc_feature 1..21: note = oligonucleotide
  source 1..21: mol_type = other RNA
cagcttctgttagccactgat

SEQ ID NO: 208 - RNA, 21 nt, synthetic construct
  misc_feature 1..21: note = oligonucleotide
  source 1..21: mol_type = other RNA
agcttctgttagccactgatt

SEQ ID NO: 209 - RNA, 22 nt, synthetic construct
  misc_feature 1..22: note = oligonucleotide
  source 1..22: mol_type = other RNA
cagcttctgttagccactgatt

SEQ ID NO: 210 - RNA, 22 nt, synthetic construct
  misc_feature 1..22: note = oligonucleotide
  source 1..22: mol_type = other RNA
agcttctgttagccactgatta

SEQ ID NO: 211 - RNA, 23 nt, synthetic construct
  misc_feature 1..23: note = oligonucleotide
  source 1..23: mol_type = other RNA
cagcttctgttagccactgatta

SEQ ID NO: 212 - RNA, 23 nt, synthetic construct
  misc_feature 1..23: note = oligonucleotide
  source 1..23: mol_type = other RNA
agcttctgttagccactgattaa

SEQ ID NO: 213 - RNA, 24 nt, synthetic construct
  misc_feature 1..24: note = oligonucleotide
  source 1..24: mol_type = other RNA
cagcttctgttagccactgattaa

SEQ ID NO: 214 - RNA, 24 nt, synthetic construct
  misc_feature 1..24: note = oligonucleotide
  source 1..24: mol_type = other RNA
agcttctgttagccactgattaaa

SEQ ID NO: 215 - RNA, 25 nt, synthetic construct
  misc_feature 1..25: note = oligonucleotide
  source 1..25: mol_type = other RNA
cagcttctgttagccactgattaaa

SEQ ID NO: 216 - RNA, 24 nt, synthetic construct
  misc_feature 1..24: note = oligonucleotide
  source 1..24: mol_type = other RNA
agcttctgttagccactgattaaa

SEQ ID NO: 217 - RNA, 20 nt, synthetic construct
  misc_feature 1..20: note = oligonucleotide
  source 1..20: mol_type = other RNA
agcttctgttagccactgat

SEQ ID NO: 218 - RNA, 20 nt, synthetic construct
  misc_feature 1..20: note = oligonucleotide
  source 1..20: mol_type = other RNA
gcttctgttagccactgatt

SEQ ID NO: 219 - RNA, 21 nt, synthetic construct
  misc_feature 1..21: note = oligonucleotide
  source 1..21: mol_type = other RNA
agcttctgttagccactgatt

SEQ ID NO: 220 - RNA, 21 nt, synthetic construct
  misc_feature 1..21: note = oligonucleotide
  source 1..21: mol_type = other RNA
gcttctgttagccactgatta

SEQ ID NO: 221 - RNA, 22 nt, synthetic construct
  misc_feature 1..22: note = oligonucleotide
  source 1..22: mol_type = other RNA
agcttctgttagccactgatta

SEQ ID NO: 222 - RNA, 22 nt, synthetic construct
  misc_feature 1..22: note = oligonucleotide
  source 1..22: mol_type = other RNA
gcttctgttagccactgattaa

SEQ ID NO: 223 - RNA, 23 nt, synthetic construct
  misc_feature 1..23: note = oligonucleotide
  source 1..23: mol_type = other RNA
agcttctgttagccactgattaa

SEQ ID NO: 224 - RNA, 23 nt, synthetic construct
  misc_feature 1..23: note = oligonucleotide
  source 1..23: mol_type = other RNA
gcttctgttagccactgattaaa

SEQ ID NO: 225 - RNA, 24 nt, synthetic construct
  misc_feature 1..24: note = oligonucleotide
  source 1..24: mol_type = other RNA
agcttctgttagccactgattaaa

SEQ ID NO: 226 - RNA, 23 nt, synthetic construct
  misc_feature 1..23: note = oligonucleotide
  source 1..23: mol_type = other RNA
gcttctgttagccactgattaaa

SEQ ID NO: 227 - RNA, 23 nt, synthetic construct
  misc_feature 1..23: note = oligonucleotide
  source 1..23: mol_type = other RNA
ccatttgtatttagcatgttccc

SEQ ID NO: 228 - RNA, 20 nt, synthetic construct
  misc_feature 1..20: note = oligonucleotide
  source 1..20: mol_type = other RNA
agataccatttgtatttagc

SEQ ID NO: 229 - RNA, 19 nt, synthetic construct
  misc_feature 1..19: note = oligonucleotide
  source 1..19: mol_type = other RNA
gccatttctcaacagatct

SEQ ID NO: 230 - RNA, 23 nt, synthetic construct
  misc_feature 1..23: note = oligonucleotide
  source 1..23: mol_type = other RNA
gccatttctcaacagatctgtca

SEQ ID NO: 231 - RNA, 23 nt, synthetic construct
  misc_feature 1..23: note = oligonucleotide
  source 1..23: mol_type = other RNA
attctcaggaatttgtgtctttc

SEQ ID NO: 232 - RNA, 21 nt, synthetic construct
  misc_feature 1..21: note = oligonucleotide
  source 1..21: mol_type = other RNA
tctcaggaatttgtgtctttc

SEQ ID NO: 233 - RNA, 18 nt, synthetic construct
  misc_feature 1..18: note = oligonucleotide
  source 1..18: mol_type = other RNA
gttcagcttctgttagcc

SEQ ID NO: 234 - RNA, 21 nt, synthetic construct
  misc_feature 1..21: note = oligonucleotide
  source 1..21: mol_type = other RNA
ctgattaaatatctttatatc

SEQ ID NO: 235 - RNA, 18 nt, synthetic construct
  misc_feature 1..18: note = oligonucleotide
  source 1..18: mol_type = other RNA
gccgccatttctcaacag

SEQ ID NO: 236 - RNA, 18 nt, synthetic construct
  misc_feature 1..18: note = oligonucleotide
  source 1..18: mol_type = other RNA
gtatttagcatgttccca

SEQ ID NO: 237 - RNA, 18 nt, synthetic construct
  misc_feature 1..18: note = oligonucleotide
  source 1..18: mol_type = other RNA
caggaatttgtgtctttc

SEQ ID NO: 238 - RNA, 25 nt, synthetic construct
  misc_feature 1..25: note = oligonucleotide
  source 1..25: mol_type = other RNA
gcttttcttttagttgctgctcttt

SEQ ID NO: 239 - RNA, 25 nt, synthetic construct
  misc_feature 1..25: note = oligonucleotide
  source 1..25: mol_type = other RNA
cttttcttttagttgctgctctttt

SEQ ID NO: 240 - RNA, 25 nt, synthetic construct
  misc_feature 1..25: note = oligonucleotide
  source 1..25: mol_type = other RNA
ttttcttttagttgctgctcttttc

SEQ ID NO: 241 - RNA, 25 nt, synthetic construct
  misc_feature 1..25: note = oligonucleotide
  source 1..25: mol_type = other RNA
tttcttttagttgctgctcttttcc

SEQ ID NO: 242 - RNA, 25 nt, synthetic construct
  misc_feature 1..25: note = oligonucleotide
  source 1..25: mol_type = other RNA
ttcttttagttgctgctcttttcca

SEQ ID NO: 243 - RNA, 25 nt, synthetic construct
  misc_feature 1..25: note = oligonucleotide
  source 1..25: mol_type = other RNA
tcttttagttgctgctcttttccag

SEQ ID NO: 244 - RNA, 25 nt, synthetic construct
  misc_feature 1..25: note = oligonucleotide
  source 1..25: mol_type = other RNA
cttttagttgctgctcttttccagg

SEQ ID NO: 245 - RNA, 25 nt, synthetic construct
  misc_feature 1..25: note = oligonucleotide
  source 1..25: mol_type = other RNA
ttttagttgctgctcttttccaggt

SEQ ID NO: 246 - RNA, 25 nt, synthetic construct
  misc_feature 1..25: note = oligonucleotide
  source 1..25: mol_type = other RNA
tttagttgctgctcttttccaggtt

SEQ ID NO: 247 - RNA, 25 nt, synthetic construct
  misc_feature 1..25: note = oligonucleotide
  source 1..25: mol_type = other RNA
ttagttgctgctcttttccaggttc

SEQ ID NO: 248 - RNA, 25 nt, synthetic construct
  misc_feature 1..25: note = oligonucleotide
  source 1..25: mol_type = other RNA
tagttgctgctcttttccaggttca

SEQ ID NO: 249 - RNA, 25 nt, synthetic construct
  misc_feature 1..25: note = oligonucleotide
  source 1..25: mol_type = other RNA
agttgctgctcttttccaggttcaa

SEQ ID NO: 250 - RNA, 25 nt, synthetic construct
  misc_feature 1..25: note = oligonucleotide
  source 1..25: mol_type = other RNA
gttgctgctcttttccaggttcaag

SEQ ID NO: 251 - RNA, 25 nt, synthetic construct
  misc_feature 1..25: note = oligonucleotide
  source 1..25: mol_type = other RNA
ttgctgctcttttccaggttcaagt

SEQ ID NO: 252 - RNA, 25 nt, synthetic construct
  misc_feature 1..25: note = oligonucleotide
  source 1..25: mol_type = other RNA
tgctgctcttttccaggttcaagtg

SEQ ID NO: 253 - RNA, 25 nt, synthetic construct
  misc_feature 1..25: note = oligonucleotide
  source 1..25: mol_type = other RNA
gctgctcttttccaggttcaagtgg

SEQ ID NO: 254 - RNA, 25 nt, synthetic construct
  misc_feature 1..25: note = oligonucleotide
  source 1..25: mol_type = other RNA
ctgctcttttccaggttcaagtggg

SEQ ID NO: 255 - RNA, 25 nt, synthetic construct
  misc_feature 1..25: note = oligonucleotide
  source 1..25: mol_type = other RNA
tgctcttttccaggttcaagtggga

SEQ ID NO: 256 - RNA, 25 nt, synthetic construct
  misc_feature 1..25: note = oligonucleotide
  source 1..25: mol_type = other RNA
gctcttttccaggttcaagtgggac

SEQ ID NO: 257 - RNA, 25 nt, synthetic construct
  misc_feature 1..25: note = oligonucleotide
  source 1..25: mol_type = other RNA
ctcttttccaggttcaagtgggata

SEQ ID NO: 258 - RNA, 25 nt, synthetic construct
  misc_feature 1..25: note = oligonucleotide
  source 1..25: mol_type = other RNA
tcttttccaggttcaagtgggatac

SEQ ID NO: 259 - RNA, 20 nt, synthetic construct
  misc_feature 1..20: note = oligonucleotide
  source 1..20: mol_type = other RNA
tcttttccaggttcaagtgg

SEQ ID NO: 260 - RNA, 25 nt, synthetic construct
  misc_feature 1..25: note = oligonucleotide
  source 1..25: mol_type = other RNA
cttttccaggttcaagtgggatact

SEQ ID NO: 261 - RNA, 25 nt, synthetic construct
  misc_feature 1..25: note = oligonucleotide
  source 1..25: mol_type = other RNA
ttttccaggttcaagtgggatacta

SEQ ID NO: 262 - RNA, 25 nt, synthetic construct
  misc_feature 1..25: note = oligonucleotide
  source 1..25: mol_type = other RNA
tttccaggttcaagtgggatactag

SEQ ID NO: 263 - RNA, 25 nt, synthetic construct
  misc_feature 1..25: note = oligonucleotide
  source 1..25: mol_type = other RNA
ttccaggttcaagtgggatactagc

SEQ ID NO: 264 - RNA, 25 nt, synthetic construct
  misc_feature 1..25: note = oligonucleotide
  source 1..25: mol_type = other RNA
tccaggttcaagtgggatactagca

SEQ ID NO: 265 - RNA, 25 nt, synthetic construct
  misc_feature 1..25: note = oligonucleotide
  source 1..25: mol_type = other RNA
ccaggttcaagtgggatactagcaa

SEQ ID NO: 266 - RNA, 25 nt, synthetic construct
  misc_feature 1..25: note = oligonucleotide
  source 1..25: mol_type = other RNA
caggttcaagtgggatactagcaat

SEQ ID NO: 267 - RNA, 25 nt, synthetic construct
  misc_feature 1..25: note = oligonucleotide
  source 1..25: mol_type = other RNA
aggttcaagtgggatactagcaatg

SEQ ID NO: 268 - RNA, 25 nt, synthetic construct
  misc_feature 1..25: note = oligonucleotide
  source 1..25: mol_type = other RNA
ggttcaagtgggatactagcaatgt

SEQ ID NO: 269 - RNA, 25 nt, synthetic construct
  misc_feature 1..25: note = oligonucleotide
  source 1..25: mol_type = other RNA
gttcaagtgggatactagcaatgtt

SEQ ID NO: 270 - RNA, 25 nt, synthetic construct
  misc_feature 1..25: note = oligonucleotide
  source 1..25: mol_type = other RNA
ttcaagtgggatactagcaatgtta

SEQ ID NO: 271 - RNA, 25 nt, synthetic construct
  misc_feature 1..25: note = oligonucleotide
  source 1..25: mol_type = other RNA
tcaagtgggatactagcaatgttat

SEQ ID NO: 272 - RNA, 25 nt, synthetic construct
  misc_feature 1..25: note = oligonucleotide
  source 1..25: mol_type = other RNA
caagtgggatactagcaatgttatc

SEQ ID NO: 273 - RNA, 25 nt, synthetic construct
  misc_feature 1..25: note = oligonucleotide
  source 1..25: mol_type = other RNA
aagtgggatactagcaatgttatct

SEQ ID NO: 274 - RNA, 25 nt, synthetic construct
  misc_feature 1..25: note = oligonucleotide
  source 1..25: mol_type = other RNA
agtgggatactagcaatgttatctg

SEQ ID NO: 275 - RNA, 25 nt, synthetic construct
  misc_feature 1..25: note = oligonucleotide
  source 1..25: mol_type = other RNA
gtgggatactagcaatgttatctgc

SEQ ID NO: 276 - RNA, 25 nt, synthetic construct
  misc_feature 1..25: note = oligonucleotide
  source 1..25: mol_type = other RNA
tgggatactagcaatgttatctgct

SEQ ID NO: 277 - RNA, 25 nt, synthetic construct
  misc_feature 1..25: note = oligonucleotide
  source 1..25: mol_type = other RNA
gggatactagcaatgttatctgctt

SEQ ID NO: 278 - RNA, 25 nt, synthetic construct
  misc_feature 1..25: note = oligonucleotide
  source 1..25: mol_type = other RNA
ggatactagcaatgttatctgcttc

SEQ ID NO: 279 - RNA, 25 nt, synthetic construct
  misc_feature 1..25: note = oligonucleotide
  source 1..25: mol_type = other RNA
gatactagcaatgttatctgcttcc

SEQ ID NO: 280 - RNA, 25 nt, synthetic construct
  misc_feature 1..25: note = oligonucleotide
  source 1..25: mol_type = other RNA
atactagcaatgttatctgcttcct

SEQ ID NO: 281 - RNA, 25 nt, synthetic construct
  misc_feature 1..25: note = oligonucleotide
  source 1..25: mol_type = other RNA
tactagcaatgttatctgcttcctc

SEQ ID NO: 282 - RNA, 25 nt, synthetic construct
  misc_feature 1..25: note = oligonucleotide
  source 1..25: mol_type = other RNA
actagcaatgttatctgcttcctcc

SEQ ID NO: 283 - RNA, 25 nt, synthetic construct
  misc_feature 1..25: note = oligonucleotide
  source 1..25: mol_type = other RNA
ctagcaatgttatctgcttcctcca

SEQ ID NO: 284 - RNA, 25 nt, synthetic construct
  misc_feature 1..25: note = oligonucleotide
  source 1..25: mol_type = other RNA
tagcaatgttatctgcttcctccaa

SEQ ID NO: 285 - RNA, 25 nt, synthetic construct
  misc_feature 1..25: note = oligonucleotide
  source 1..25: mol_type = other RNA
agcaatgttatctgcttcctccaac

SEQ ID NO: 286 - RNA, 25 nt, synthetic construct
  misc_feature 1..25: note = oligonucleotide
  source 1..25: mol_type = other RNA
gcaatgttatctgcttcctccaacc

SEQ ID NO: 287 - RNA, 25 nt, synthetic construct
  misc_feature 1..25: note = oligonucleotide
  source 1..25: mol_type = other RNA
caatgttatctgcttcctccaacca

SEQ ID NO: 288 - RNA, 25 nt, synthetic construct
  misc_feature 1..25: note = oligonucleotide
  source 1..25: mol_type = other RNA
aatgttatctgcttcctccaaccat

SEQ ID NO: 289 - RNA, 25 nt, synthetic construct
  misc_feature 1..25: note = oligonucleotide
  source 1..25: mol_type = other RNA
atgttatctgcttcctccaaccata

SEQ ID NO: 290 - RNA, 25 nt, synthetic construct
  misc_feature 1..25: note = oligonucleotide
  source 1..25: mol_type = other RNA
tgttatctgcttcctccaaccataa

SEQ ID NO: 291 - RNA, 25 nt, synthetic construct
  misc_feature 1..25: note = oligonucleotide
  source 1..25: mol_type = other RNA
agcctcttgattgctggtcttgttt

SEQ ID NO: 292 - RNA, 25 nt, synthetic construct
  misc_feature 1..25: note = oligonucleotide
  source 1..25: mol_type = other RNA
gcctcttgattgctggtcttgtttt

SEQ ID NO: 293 - RNA, 25 nt, synthetic construct
  misc_feature 1..25: note = oligonucleotide
  source 1..25: mol_type = other RNA
cctcttgattgctggtcttgttttt

SEQ ID NO: 294 - RNA, 20 nt, synthetic construct
  misc_feature 1..20: note = oligonucleotide
  source 1..20: mol_type = other RNA
cctcttgattgctggtcttg

SEQ ID NO: 295 - RNA, 25 nt, synthetic construct
  misc_feature 1..25: note = oligonucleotide
  source 1..25: mol_type = other RNA
ctcttgattgctggtcttgtttttc

SEQ ID NO: 296 - RNA, 25 nt, synthetic construct
  misc_feature 1..25: note = oligonucleotide
  source 1..25: mol_type = other RNA
tcttgattgctggtcttgtttttca

SEQ ID NO: 297 - RNA, 25 nt, synthetic construct
  misc_feature 1..25: note = oligonucleotide
  source 1..25: mol_type = other RNA
cttgattgctggtcttgtttttcaa

SEQ ID NO: 298 - RNA, 25 nt, synthetic construct
  misc_feature 1..25: note = oligonucleotide
  source 1..25: mol_type = other RNA
ttgattgctggtcttgtttttcaaa

SEQ ID NO: 299 - RNA, 25 nt, synthetic construct
  misc_feature 1..25: note = oligonucleotide
  source 1..25: mol_type = other RNA
tgattgctggtcttgtttttcaaat

SEQ ID NO: 300 - RNA, 25 nt, synthetic construct
  misc_feature 1..25: note = oligonucleotide
  source 1..25: mol_type = other RNA
gattgctggtcttgtttttcaaatt

SEQ ID NO: 301 - RNA, 20 nt, synthetic construct
  misc_feature 1..20: note = oligonucleotide
  source 1..20: mol_type = other RNA
gattgctggtcttgtttttc

SEQ ID NO: 302 - RNA, 25 nt, synthetic construct
  misc_feature 1..25: note = oligonucleotide
  source 1..25: mol_type = other RNA
attgctggtcttgtttttcaaattt

SEQ ID NO: 303 - RNA, 25 nt, synthetic construct
  misc_feature 1..25: note = oligonucleotide
  source 1..25: mol_type = other RNA
ttgctggtcttgtttttcaaatttt

SEQ ID NO: 304 - RNA, 25 nt, synthetic construct
  misc_feature 1..25: note = oligonucleotide
  source 1..25: mol_type = other RNA
tgctggtcttgtttttcaaattttg

SEQ ID NO: 305 - RNA, 25 nt, synthetic construct
  misc_feature 1..25: note = oligonucleotide
  source 1..25: mol_type = other RNA
gctggtcttgtttttcaaattttgg

SEQ ID NO: 306 - RNA, 25 nt, synthetic construct
  misc_feature 1..25: note = oligonucleotide
  source 1..25: mol_type = other RNA
ctggtcttgtttttcaaattttggg

SEQ ID NO: 307 - RNA, 25 nt, synthetic construct
  misc_feature 1..25: note = oligonucleotide
  source 1..25: mol_type = other RNA
tggtcttgtttttcaaattttgggc

SEQ ID NO: 308 - RNA, 25 nt, synthetic construct
  misc_feature 1..25: note = oligonucleotide
  source 1..25: mol_type = other RNA
ggtcttgtttttcaaattttgggca

SEQ ID NO: 309 - RNA, 25 nt, synthetic construct
  misc_feature 1..25: note = oligonucleotide
  source 1..25: mol_type = other RNA
gtcttgtttttcaaattttgggcag

SEQ ID NO: 310 - RNA, 25 nt, synthetic construct
  misc_feature 1..25: note = oligonucleotide
  source 1..25: mol_type = other RNA
tcttgtttttcaaattttgggcagc

SEQ ID NO: 311 - RNA, 25 nt, synthetic construct
  misc_feature 1..25: note = oligonucleotide
  source 1..25: mol_type = other RNA
cttgtttttcaaattttgggcagcg

SEQ ID NO: 312 - RNA, 25 nt, synthetic construct
  misc_feature 1..25: note = oligonucleotide
  source 1..25: mol_type = other RNA
ttgtttttcaaattttgggcagcgg

SEQ ID NO: 313 - RNA, 25 nt, synthetic construct
  misc_feature 1..25: note = oligonucleotide
  source 1..25: mol_type = other RNA
tgtttttcaaattttgggcagcggt

SEQ ID NO: 314 - RNA, 25 nt, synthetic construct
  misc_feature 1..25: note = oligonucleotide
  source 1..25: mol_type = other RNA
gtttttcaaattttgggcagcggta

SEQ ID NO: 315 - RNA, 25 nt, synthetic construct
  misc_feature 1..25: note = oligonucleotide
  source 1..25: mol_type = other RNA
tttttcaaattttgggcagcggtaa

SEQ ID NO: 316 - RNA, 25 nt, synthetic construct
  misc_feature 1..25: note = oligonucleotide
  source 1..25: mol_type = other RNA
ttttcaaattttgggcagcggtaat

SEQ ID NO: 317 - RNA, 25 nt, synthetic construct
  misc_feature 1..25: note = oligonucleotide
  source 1..25: mol_type = other RNA
tttcaaattttgggcagcggtaatg

SEQ ID NO: 318 - RNA, 25 nt, synthetic construct
  misc_feature 1..25: note = oligonucleotide
  source 1..25: mol_type = other RNA
ttcaaattttgggcagcggtaatga

SEQ ID NO: 319 - RNA, 25 nt, synthetic construct
  misc_feature 1..25: note = oligonucleotide
  source 1..25: mol_type = other RNA
tcaaattttgggcagcggtaatgag

SEQ ID NO: 320 - RNA, 25 nt, synthetic construct
  misc_feature 1..25: note = oligonucleotide
  source 1..25: mol_type = other RNA
caaattttgggcagcggtaatgagt

SEQ ID NO: 321 - RNA, 25 nt, synthetic construct
  misc_feature 1..25: note = oligonucleotide
  source 1..25: mol_type = other RNA
aaattttgggcagcggtaatgagtt

SEQ ID NO: 322 - RNA, 25 nt, synthetic construct
  misc_feature 1..25: note = oligonucleotide
  source 1..25: mol_type = other RNA
aattttgggcagcggtaatgagttc

SEQ ID NO: 323 - RNA, 25 nt, synthetic construct
  misc_feature 1..25: note = oligonucleotide
  source 1..25: mol_type = other RNA
attttgggcagcggtaatgagttct

SEQ ID NO: 324 - RNA, 25 nt, synthetic construct
  misc_feature 1..25: note = oligonucleotide
  source 1..25: mol_type = other RNA
ttttgggcagcggtaatgagttctt

SEQ ID NO: 325 - RNA, 25 nt, synthetic construct
  misc_feature 1..25: note = oligonucleotide
  source 1..25: mol_type = other RNA
tttgggcagcggtaatgagttcttc

SEQ ID NO: 326 - RNA, 25 nt, synthetic construct
  misc_feature 1..25: note = oligonucleotide
  source 1..25: mol_type = other RNA
ttgggcagcggtaatgagttcttcc

SEQ ID NO: 327 - RNA, 25 nt, synthetic construct
  misc_feature 1..25: note = oligonucleotide
  source 1..25: mol_type = other RNA
tgggcagcggtaatgagttcttcca

SEQ ID NO: 328 - RNA, 25 nt, synthetic construct
  misc_feature 1..25: note = oligonucleotide
  source 1..25: mol_type = other RNA
gggcagcggtaatgagttcttccaa

SEQ ID NO: 329 - RNA, 25 nt, synthetic construct
  misc_feature 1..25: note = oligonucleotide
  source 1..25: mol_type = other RNA
ggcagcggtaatgagttcttccaac

SEQ ID NO: 330 - RNA, 25 nt, synthetic construct
  misc_feature 1..25: note = oligonucleotide
  source 1..25: mol_type = other RNA
gcagcggtaatgagttcttccaact

SEQ ID NO: 331 - RNA, 25 nt, synthetic construct
  misc_feature 1..25: note = oligonucleotide
  source 1..25: mol_type = other RNA
cagcggtaatgagttcttccaactg

SEQ ID NO: 332 - RNA, 25 nt, synthetic construct
  misc_feature 1..25: note = oligonucleotide
  source 1..25: mol_type = other RNA
agcggtaatgagttcttccaactgg

SEQ ID NO: 333 - RNA, 25 nt, synthetic construct
  misc_feature 1..25: note = oligonucleotide
  source 1..25: mol_type = other RNA
gcggtaatgagttcttccaactggg

SEQ ID NO: 334 - RNA, 25 nt, synthetic construct
  misc_feature 1..25: note = oligonucleotide
  source 1..25: mol_type = other RNA
cggtaatgagttcttccaactgggg

SEQ ID NO: 335 - RNA, 25 nt, synthetic construct
  misc_feature 1..25: note = oligonucleotide
  source 1..25: mol_type = other RNA
ggtaatgagttcttccaactgggga

SEQ ID NO: 336 - RNA, 22 nt, synthetic construct
  misc_feature 1..22: note = oligonucleotide
  source 1..22: mol_type = other RNA
ggtaatgagttcttccaactgg

SEQ ID NO: 337 - RNA, 25 nt, synthetic construct
  misc_feature 1..25: note = oligonucleotide
  source 1..25: mol_type = other RNA
gtaatgagttcttccaactggggac

SEQ ID NO: 338 - RNA, 25 nt, synthetic construct
  misc_feature 1..25: note = oligonucleotide
  source 1..25: mol_type = other RNA
taatgagttcttccaactggggacg

SEQ ID NO: 339 - RNA, 25 nt, synthetic construct
  misc_feature 1..25: note = oligonucleotide
  source 1..25: mol_type = other RNA
aatgagttcttccaactggggacgc

SEQ ID NO: 340 - RNA, 25 nt, synthetic construct
  misc_feature 1..25: note = oligonucleotide
  source 1..25: mol_type = other RNA
atgagttcttccaactggggacgcc

SEQ ID NO: 341 - RNA, 25 nt, synthetic construct
  misc_feature 1..25: note = oligonucleotide
  source 1..25: mol_type = other RNA
tgagttcttccaactggggacgcct

SEQ ID NO: 342 - RNA, 25 nt, synthetic construct
  misc_feature 1..25: note = oligonucleotide
  source 1..25: mol_type = other RNA
gagttcttccaactggggacgcctc

SEQ ID NO: 343 - RNA, 25 nt, synthetic construct
  misc_feature 1..25: note = oligonucleotide
  source 1..25: mol_type = other RNA
agttcttccaactggggacgcctct

SEQ ID NO: 344 - RNA, 25 nt, synthetic construct
  misc_feature 1..25: note = oligonucleotide
  source 1..25: mol_type = other RNA
gttcttccaactggggacgcctctg

SEQ ID NO: 345 - RNA, 25 nt, synthetic construct
  misc_feature 1..25: note = oligonucleotide
  source 1..25: mol_type = other RNA
ttcttccaactggggacgcctctgt

SEQ ID NO: 346 - RNA, 25 nt, synthetic construct
  misc_feature 1..25: note = oligonucleotide
  source 1..25: mol_type = other RNA
tcttccaactggggacgcctctgtt

SEQ ID NO: 347 - RNA, 25 nt, synthetic construct
  misc_feature 1..25: note = oligonucleotide
  source 1..25: mol_type = other RNA
cttccaactggggacgcctctgttc

SEQ ID NO: 348 - RNA, 25 nt, synthetic construct
  misc_feature 1..25: note = oligonucleotide
  source 1..25: mol_type = other RNA
ttccaactggggacgcctctgttcc

SEQ ID NO: 349 - RNA, 25 nt, synthetic construct
  misc_feature 1..25: note = oligonucleotide
  source 1..25: mol_type = other RNA
tccaactggggacgcctctgttcca

SEQ ID NO: 350 - RNA, 25 nt, synthetic construct
  misc_feature 1..25: note = oligonucleotide
  source 1..25: mol_type = other RNA
ccaactggggacgcctctgttccaa

SEQ ID NO: 351 - RNA, 25 nt, synthetic construct
  misc_feature 1..25: note = oligonucleotide
  source 1..25: mol_type = other RNA
caactggggacgcctctgttccaaa

SEQ ID NO: 352 - RNA, 25 nt, synthetic construct
  misc_feature 1..25: note = oligonucleotide
  source 1..25: mol_type = other RNA
aactggggacgcctctgttccaaat

SEQ ID NO: 353 - RNA, 25 nt, synthetic construct
  misc_feature 1..25: note = oligonucleotide
  source 1..25: mol_type = other RNA
actggggacgcctctgttccaaatc

SEQ ID NO: 354 - RNA, 25 nt, synthetic construct
  misc_feature 1..25: note = oligonucleotide
  source 1..25: mol_type = other RNA
ctggggacgcctctgttccaaatcc

SEQ ID NO: 355 - RNA, 25 nt, synthetic construct
  misc_feature 1..25: note = oligonucleotide
  source 1..25: mol_type = other RNA
tggggacgcctctgttccaaatcct

SEQ ID NO: 356 - RNA, 25 nt, synthetic construct
  misc_feature 1..25: note = oligonucleotide
  source 1..25: mol_type = other RNA
ggggacgcctctgttccaaatcctg

SEQ ID NO: 357 - RNA, 25 nt, synthetic construct
  misc_feature 1..25: note = oligonucleotide
  source 1..25: mol_type = other RNA
gggacgcctctgttccaaatcctgc

SEQ ID NO: 358 - RNA, 25 nt, synthetic construct
  misc_feature 1..25: note = oligonucleotide
  source 1..25: mol_type = other RNA
ggacgcctctgttccaaatcctgca

SEQ ID NO: 359 - RNA, 25 nt, synthetic construct
  misc_feature 1..25: note = oligonucleotide
  source 1..25: mol_type = other RNA
gacgcctctgttccaaatcctgcat

SEQ ID NO: 360 - RNA, 25 nt, synthetic construct
  misc_feature 1..25: note = oligonucleotide
  source 1..25: mol_type = other RNA
ccaatagtggtcagtccaggagcta

SEQ ID NO: 361 - RNA, 25 nt, synthetic construct
  misc_feature 1..25: note = oligonucleotide
  source 1..25: mol_type = other RNA
caatagtggtcagtccaggagctag

SEQ ID NO: 362 - RNA, 25 nt, synthetic construct
  misc_feature 1..25: note = oligonucleotide
  source 1..25: mol_type = other RNA
aatagtggtcagtccaggagctagg

SEQ ID NO: 363 - RNA, 25 nt, synthetic construct
  misc_feature 1..25: note = oligonucleotide
  source 1..25: mol_type = other RNA
atagtggtcagtccaggagctaggt

SEQ ID NO: 364 - RNA, 21 nt, synthetic construct
  misc_feature 1..21: note = oligonucleotide
  source 1..21: mol_type = other RNA
atagtggtcagtccaggagct

SEQ ID NO: 365 - RNA, 25 nt, synthetic construct
  misc_feature 1..25: note = oligonucleotide
  source 1..25: mol_type = other RNA
tagtggtcagtccaggagctaggtc

SEQ ID NO: 366 - RNA, 25 nt, synthetic construct
  misc_feature 1..25: note = oligonucleotide
  source 1..25: mol_type = other RNA
agtggtcagtccaggagctaggtca

SEQ ID NO: 367 - RNA, 25 nt, synthetic construct
  misc_feature 1..25: note = oligonucleotide
  source 1..25: mol_type = other RNA
gtggtcagtccaggagctaggtcag

SEQ ID NO: 368 - RNA, 25 nt, synthetic construct
  misc_feature 1..25: note = oligonucleotide
  source 1..25: mol_type = other RNA
tggtcagtccaggagctaggtcagg

SEQ ID NO: 369 - RNA, 25 nt, synthetic construct
  misc_feature 1..25: note = oligonucleotide
  source 1..25: mol_type = other RNA
ggtcagtccaggagctaggtcaggc

SEQ ID NO: 370 - RNA, 25 nt, synthetic construct
  misc_feature 1..25: note = oligonucleotide
  source 1..25: mol_type = other RNA
gtcagtccaggagctaggtcaggct

SEQ ID NO: 371 - RNA, 25 nt, synthetic construct
  misc_feature 1..25: note = oligonucleotide
  source 1..25: mol_type = other RNA
tcagtccaggagctaggtcaggctg

SEQ ID NO: 372 - RNA, 25 nt, synthetic construct
  misc_feature 1..25: note = oligonucleotide
  source 1..25: mol_type = other RNA
cagtccaggagctaggtcaggctgc

SEQ ID NO: 373 - RNA, 25 nt, synthetic construct
  misc_feature 1..25: note = oligonucleotide
  source 1..25: mol_type = other RNA
agtccaggagctaggtcaggctgct

SEQ ID NO: 374 - RNA, 25 nt, synthetic construct
  misc_feature 1..25: note = oligonucleotide
  source 1..25: mol_type = other RNA
gtccaggagctaggtcaggctgctt

SEQ ID NO: 375 - RNA, 25 nt, synthetic construct
  misc_feature 1..25: note = oligonucleotide
  source 1..25: mol_type = other RNA
tccaggagctaggtcaggctgcttt

SEQ ID NO: 376 - RNA, 25 nt, synthetic construct
  misc_feature 1..25: note = oligonucleotide
  source 1..25: mol_type = other RNA
ccaggagctaggtcaggctgctttg

SEQ ID NO: 377 - RNA, 25 nt, synthetic construct
  misc_feature 1..25: note = oligonucleotide
  source 1..25: mol_type = other RNA
caggagctaggtcaggctgctttgc

SEQ ID NO: 378 - RNA, 25 nt, synthetic construct
  misc_feature 1..25: note = oligonucleotide
  source 1..25: mol_type = other RNA
aggagctaggtcaggctgctttgcc

SEQ ID NO: 379 - RNA, 25 nt, synthetic construct
  misc_feature 1..25: note = oligonucleotide
  source 1..25: mol_type = other RNA
ggagctaggtcaggctgctttgccc

SEQ ID NO: 380 - RNA, 25 nt, synthetic construct
  misc_feature 1..25: note = oligonucleotide
  source 1..25: mol_type = other RNA
gagctaggtcaggctgctttgccct

SEQ ID NO: 381 - RNA, 25 nt, synthetic construct
  misc_feature 1..25: note = oligonucleotide
  source 1..25: mol_type = other RNA
agctaggtcaggctgctttgccctc

SEQ ID NO: 382 - RNA, 25 nt, synthetic construct
  misc_feature 1..25: note = oligonucleotide
  source 1..25: mol_type = other RNA
gctaggtcaggctgctttgccctca

SEQ ID NO: 383 - RNA, 25 nt, synthetic construct
  misc_feature 1..25: note = oligonucleotide
  source 1..25: mol_type = other RNA
ctcagctcttgaagtaaacggttta

SEQ ID NO: 384 - RNA, 25 nt, synthetic construct
  misc_feature 1..25: note = oligonucleotide
  source 1..25: mol_type = other RNA
cagctcttgaagtaaacggtttacc

SEQ ID NO: 385 - RNA, 25 nt, synthetic construct
  misc_feature 1..25: note = oligonucleotide
  source 1..25: mol_type = other RNA
gctcttgaagtaaacggtttaccgc

SEQ ID NO: 386 - RNA, 25 nt, synthetic construct
  misc_feature 1..25: note = oligonucleotide
  source 1..25: mol_type = other RNA
ctaggtcaggctgctttgccctcag

SEQ ID NO: 387 - RNA, 25 nt, synthetic construct
  misc_feature 1..25: note = oligonucleotide
  source 1..25: mol_type = other RNA
taggtcaggctgctttgccctcagc

SEQ ID NO: 388 - RNA, 25 nt, synthetic construct
  misc_feature 1..25: note = oligonucleotide
  source 1..25: mol_type = other RNA
aggtcaggctgctttgccctcagct

SEQ ID NO: 389 - RNA, 25 nt, synthetic construct
  misc_feature 1..25: note = oligonucleotide
  source 1..25: mol_type = other RNA
ggtcaggctgctttgccctcagctc

SEQ ID NO: 390 - RNA, 25 nt, synthetic construct
  misc_feature 1..25: note = oligonucleotide
  source 1..25: mol_type = other RNA
gtcaggctgctttgccctcagctct

SEQ ID NO: 391 - RNA, 25 nt, synthetic construct
  misc_feature 1..25: note = oligonucleotide
  source 1..25: mol_type = other RNA
tcaggctgctttgccctcagctctt

SEQ ID NO: 392 - RNA, 25 nt, synthetic construct
  misc_feature 1..25: note = oligonucleotide
  source 1..25: mol_type = other RNA
caggctgctttgccctcagctcttg

SEQ ID NO: 393 - RNA, 25 nt, synthetic construct
  misc_feature 1..25: note = oligonucleotide
  source 1..25: mol_type = other RNA
aggctgctttgccctcagctcttga

SEQ ID NO: 394 - RNA, 25 nt, synthetic construct
  misc_feature 1..25: note = oligonucleotide
  source 1..25: mol_type = other RNA
ggctgctttgccctcagctcttgaa

SEQ ID NO: 395 - RNA, 25 nt, synthetic construct
  misc_feature 1..25: note = oligonucleotide
  source 1..25: mol_type = other RNA
gctgctttgccctcagctcttgaag

SEQ ID NO: 396 - RNA, 25 nt, synthetic construct
  misc_feature 1..25: note = oligonucleotide
  source 1..25: mol_type = other RNA
ctgctttgccctcagctcttgaagt

SEQ ID NO: 397 - RNA, 25 nt, synthetic construct
  misc_feature 1..25: note = oligonucleotide
  source 1..25: mol_type = other RNA
tgctttgccctcagctcttgaagta

SEQ ID NO: 398 - RNA, 25 nt, synthetic construct
  misc_feature 1..25: note = oligonucleotide
  source 1..25: mol_type = other RNA
gctttgccctcagctcttgaagtaa

SEQ ID NO: 399 - RNA, 25 nt, synthetic construct
  misc_feature 1..25: note = oligonucleotide
  source 1..25: mol_type = other RNA
ctttgccctcagctcttgaagtaaa

SEQ ID NO: 400 - RNA, 25 nt, synthetic construct
  misc_feature 1..25: note = oligonucleotide
  source 1..25: mol_type = other RNA
tttgccctcagctcttgaagtaaac

SEQ ID NO: 401 - RNA, 25 nt, synthetic construct
  misc_feature 1..25: note = oligonucleotide
  source 1..25: mol_type = other RNA
ttgccctcagctcttgaagtaaacg

SEQ ID NO: 402 - RNA, 25 nt, synthetic construct
  misc_feature 1..25: note = oligonucleotide
  source 1..25: mol_type = other RNA
tgccctcagctcttgaagtaaacgg

SEQ ID NO: 403 - RNA, 25 nt, synthetic construct
  misc_feature 1..25: note = oligonucleotide
  source 1..25: mol_type = other RNA
gccctcagctcttgaagtaaacggt

SEQ ID NO: 404 - RNA, 25 nt, synthetic construct
  misc_feature 1..25: note = oligonucleotide
  source 1..25: mol_type = other RNA
ccctcagctcttgaagtaaacggtt

SEQ ID NO: 405 - RNA, 20 nt, synthetic construct
  misc_feature 1..20: note = oligonucleotide
  source 1..20: mol_type = other RNA
cctcagctcttgaagtaaac

SEQ ID NO: 406 - RNA, 21 nt, synthetic construct
  misc_feature 1..21: note = oligonucleotide
  source 1..21: mol_type = other RNA
cctcagctcttgaagtaaacg

SEQ ID NO: 407 - RNA, 20 nt, synthetic construct
  misc_feature 1..20: note = oligonucleotide
  source 1..20: mol_type = other RNA
ctcagctcttgaagtaaacg

SEQ ID NO: 408 - RNA, 25 nt, synthetic construct
  misc_feature 1..25: note = oligonucleotide
  source 1..25: mol_type = other RNA
cctcagctcttgaagtaaacggttt

SEQ ID NO: 409 - RNA, 25 nt, synthetic construct
  misc_feature 1..25: note = oligonucleotide
  source 1..25: mol_type = other RNA
tcagctcttgaagtaaacggtttac

SEQ ID NO: 410 - RNA, 25 nt, synthetic construct
  misc_feature 1..25: note = oligonucleotide
  source 1..25: mol_type = other RNA
agctcttgaagtaaacggtttaccg

SEQ ID NO: 411 - RNA, 25 nt, synthetic construct
  misc_feature 1..25: note = oligonucleotide
  source 1..25: mol_type = other RNA
ctcttgaagtaaacggtttaccgcc

SEQ ID NO: 412 - RNA, 25 nt, synthetic construct
  misc_feature 1..25: note = oligonucleotide
  source 1..25: mol_type = other RNA
ccacaggcgttgcactttgcaatgc

SEQ ID NO: 413 - RNA, 25 nt, synthetic construct
  misc_feature 1..25: note = oligonucleotide
  source 1..25: mol_type = other RNA
cacaggcgttgcactttgcaatgct

SEQ ID NO: 414 - RNA, 25 nt, synthetic construct
  misc_feature 1..25: note = oligonucleotide
  source 1..25: mol_type = other RNA
acaggcgttgcactttgcaatgctg

SEQ ID NO: 415 - RNA, 25 nt, synthetic construct
  misc_feature 1..25: note = oligonucleotide
  source 1..25: mol_type = other RNA
caggcgttgcactttgcaatgctgc

SEQ ID NO: 416 - RNA, 25 nt, synthetic construct
  misc_feature 1..25: note = oligonucleotide
  source 1..25: mol_type = other RNA
aggcgttgcactttgcaatgctgct

SEQ ID NO: 417 - RNA, 25 nt, synthetic construct
  misc_feature 1..25: note = oligonucleotide
  source 1..25: mol_type = other RNA
ggcgttgcactttgcaatgctgctg

SEQ ID NO: 418 - RNA, 25 nt, synthetic construct
  misc_feature 1..25: note = oligonucleotide
  source 1..25: mol_type = other RNA
gcgttgcactttgcaatgctgctgt

SEQ ID NO: 419 - RNA, 25 nt, synthetic construct
  misc_feature 1..25: note = oligonucleotide
  source 1..25: mol_type = other RNA
cgttgcactttgcaatgctgctgtc

SEQ ID NO: 420 - RNA, 23 nt, synthetic construct
  misc_feature 1..23: note = oligonucleotide
  source 1..23: mol_type = other RNA
cgttgcactttgcaatgctgctg

SEQ ID NO: 421 - RNA, 25 nt, synthetic construct
  misc_feature 1..25: note = oligonucleotide
  source 1..25: mol_type = other RNA
gttgcactttgcaatgctgctgtct

SEQ ID NO: 422 - RNA, 25 nt, synthetic construct
  misc_feature 1..25: note = oligonucleotide
  source 1..25: mol_type = other RNA
ttgcactttgcaatgctgctgtctt

SEQ ID NO: 423 - RNA, 25 nt, synthetic construct
  misc_feature 1..25: note = oligonucleotide
  source 1..25: mol_type = other RNA
tgcactttgcaatgctgctgtcttc

SEQ ID NO: 424 - RNA, 25 nt, synthetic construct
  misc_feature 1..25: note = oligonucleotide
  source 1..25: mol_type = other RNA
gcactttgcaatgctgctgtcttct

SEQ ID NO: 425 - RNA, 25 nt, synthetic construct
  misc_feature 1..25: note = oligonucleotide
  source 1..25: mol_type = other RNA
cactttgcaatgctgctgtcttctt

SEQ ID NO: 426 - RNA, 25 nt, synthetic construct
  misc_feature 1..25: note = oligonucleotide
  source 1..25: mol_type = other RNA
actttgcaatgctgctgtcttcttg

SEQ ID NO: 427 - RNA, 25 nt, synthetic construct
  misc_feature 1..25: note = oligonucleotide
  source 1..25: mol_type = other RNA
ctttgcaatgctgctgtcttcttgc

SEQ ID NO: 428 - RNA, 25 nt, synthetic construct
  misc_feature 1..25: note = oligonucleotide
  source 1..25: mol_type = other RNA
tttgcaatgctgctgtcttcttgct

SEQ ID NO: 429 - RNA, 25 nt, synthetic construct
  misc_feature 1..25: note = oligonucleotide
  source 1..25: mol_type = other RNA
ttgcaatgctgctgtcttcttgcta

SEQ ID NO: 430 - RNA, 25 nt, synthetic construct
  misc_feature 1..25: note = oligonucleotide
  source 1..25: mol_type = other RNA
tgcaatgctgctgtcttcttgctat

SEQ ID NO: 431 - RNA, 25 nt, synthetic construct
  misc_feature 1..25: note = oligonucleotide
  source 1..25: mol_type = other RNA
gcaatgctgctgtcttcttgctatg

SEQ ID NO: 432 - RNA, 25 nt, synthetic construct
  misc_feature 1..25: note = oligonucleotide
  source 1..25: mol_type = other RNA
caatgctgctgtcttcttgctatga

SEQ ID NO: 433 - RNA, 25 nt, synthetic construct
  misc_feature 1..25: note = oligonucleotide
  source 1..25: mol_type = other RNA
aatgctgctgtcttcttgctatgaa

SEQ ID NO: 434 - RNA, 25 nt, synthetic construct
  misc_feature 1..25: note = oligonucleotide
  source 1..25: mol_type = other RNA
atgctgctgtcttcttgctatgaat

SEQ ID NO: 435 - RNA, 25 nt, synthetic construct
  misc_feature 1..25: note = oligonucleotide
  source 1..25: mol_type = other RNA
tgctgctgtcttcttgctatgaata

SEQ ID NO: 436 - RNA, 25 nt, synthetic construct
  misc_feature 1..25: note = oligonucleotide
  source 1..25: mol_type = other RNA
gctgctgtcttcttgctatgaataa

SEQ ID NO: 437 - RNA, 25 nt, synthetic construct
  misc_feature 1..25: note = oligonucleotide
  source 1..25: mol_type = other RNA
ctgctgtcttcttgctatgaataat

SEQ ID NO: 438 - RNA, 25 nt, synthetic construct
  misc_feature 1..25: note = oligonucleotide
  source 1..25: mol_type = other RNA
tgctgtcttcttgctatgaataatg

SEQ ID NO: 439 - RNA, 25 nt, synthetic construct
  misc_feature 1..25: note = oligonucleotide
  source 1..25: mol_type = other RNA
gctgtcttcttgctatgaataatgt

SEQ ID NO: 440 - RNA, 25 nt, synthetic construct
  misc_feature 1..25: note = oligonucleotide
  source 1..25: mol_type = other RNA
ctgtcttcttgctatgaataatgtc

SEQ ID NO: 441 - RNA, 25 nt, synthetic construct
  misc_feature 1..25: note = oligonucleotide
  source 1..25: mol_type = other RNA
tgtcttcttgctatgaataatgtca

SEQ ID NO: 442 - RNA, 25 nt, synthetic construct
  misc_feature 1..25: note = oligonucleotide
  source 1..25: mol_type = other RNA
gtcttcttgctatgaataatgtcaa

SEQ ID NO: 443 - RNA, 25 nt, synthetic construct
  misc_feature 1..25: note = oligonucleotide
  source 1..25: mol_type = other RNA
tcttcttgctatgaataatgtcaat

SEQ ID NO: 444 - RNA, 25 nt, synthetic construct
  misc_feature 1..25: note = oligonucleotide
  source 1..25: mol_type = other RNA
cttcttgctatgaataatgtcaatc

SEQ ID NO: 445 - RNA, 25 nt, synthetic construct
  misc_feature 1..25: note = oligonucleotide
  source 1..25: mol_type = other RNA
ttcttgctatgaataatgtcaatcc

SEQ ID NO: 446 - RNA, 25 nt, synthetic construct
  misc_feature 1..25: note = oligonucleotide
  source 1..25: mol_type = other RNA
tcttgctatgaataatgtcaatccg

SEQ ID NO: 447 - RNA, 25 nt, synthetic construct
  misc_feature 1..25: note = oligonucleotide
  source 1..25: mol_type = other RNA
cttgctatgaataatgtcaatccga

SEQ ID NO: 448 - RNA, 25 nt, synthetic construct
  misc_feature 1..25: note = oligonucleotide
  source 1..25: mol_type = other RNA
ttgctatgaataatgtcaatccgac

SEQ ID NO: 449 - RNA, 25 nt, synthetic construct
  misc_feature 1..25: note = oligonucleotide
  source 1..25: mol_type = other RNA
tgctatgaataatgtcaatccgacc

SEQ ID NO: 450 - RNA, 25 nt, synthetic construct
  misc_feature 1..25: note = oligonucleotide
  source 1..25: mol_type = other RNA
gctatgaataatgtcaatccgacct

SEQ ID NO: 451 - RNA, 25 nt, synthetic construct
  misc_feature 1..25: note = oligonucleotide
  source 1..25: mol_type = other RNA
ctatgaataatgtcaatccgacctg

SEQ ID NO: 452 - RNA, 25 nt, synthetic construct
  misc_feature 1..25: note = oligonucleotide
  source 1..25: mol_type = other RNA
tatgaataatgtcaatccgacctga

SEQ ID NO: 453 - RNA, 25 nt, synthetic construct
  misc_feature 1..25: note = oligonucleotide
  source 1..25: mol_type = other RNA
atgaataatgtcaatccgacctgag

SEQ ID NO: 454 - RNA, 25 nt, synthetic construct
  misc_feature 1..25: note = oligonucleotide
  source 1..25: mol_type = other RNA
tgaataatgtcaatccgacctgagc

SEQ ID NO: 455 - RNA, 25 nt, synthetic construct
  misc_feature 1..25: note = oligonucleotide
  source 1..25: mol_type = other RNA
gaataatgtcaatccgacctgagct

SEQ ID NO: 456 - RNA, 25 nt, synthetic construct
  misc_feature 1..25: note = oligonucleotide
  source 1..25: mol_type = other RNA
aataatgtcaatccgacctgagctt

SEQ ID NO: 457 - RNA, 25 nt, synthetic construct
  misc_feature 1..25: note = oligonucleotide
  source 1..25: mol_type = other RNA
ataatgtcaatccgacctgagcttt

SEQ ID NO: 458 - RNA, 25 nt, synthetic construct
  misc_feature 1..25: note = oligonucleotide
  source 1..25: mol_type = other RNA
taatgtcaatccgacctgagctttg

SEQ ID NO: 459 - RNA, 25 nt, synthetic construct
  misc_feature 1..25: note = oligonucleotide
  source 1..25: mol_type = other RNA
aatgtcaatccgacctgagctttgt

SEQ ID NO: 460 - RNA, 25 nt, synthetic construct
  misc_feature 1..25: note = oligonucleotide
  source 1..25: mol_type = other RNA
atgtcaatccgacctgagctttgtt

SEQ ID NO: 461 - RNA, 25 nt, synthetic construct
  misc_feature 1..25: note = oligonucleotide
  source 1..25: mol_type = other RNA
tgtcaatccgacctgagctttgttg

SEQ ID NO: 462 - RNA, 25 nt, synthetic construct
  misc_feature 1..25: note = oligonucleotide
  source 1..25: mol_type = other RNA
gtcaatccgacctgagctttgttgt

SEQ ID NO: 463 - RNA, 25 nt, synthetic construct
  misc_feature 1..25: note = oligonucleotide
  source 1..25: mol_type = other RNA
tcaatccgacctgagctttgttgta

SEQ ID NO: 464 - RNA, 25 nt, synthetic construct
  misc_feature 1..25: note = oligonucleotide
  source 1..25: mol_type = other RNA
caatccgacctgagctttgttgtag

SEQ ID NO: 465 - RNA, 25 nt, synthetic construct
  misc_feature 1..25: note = oligonucleotide
  source 1..25: mol_type = other RNA
aatccgacctgagctttgttgtaga

SEQ ID NO: 466 - RNA, 25 nt, synthetic construct
  misc_feature 1..25: note = oligonucleotide
  source 1..25: mol_type = other RNA
atccgacctgagctttgttgtagac

SEQ ID NO: 467 - RNA, 25 nt, synthetic construct
  misc_feature 1..25: note = oligonucleotide
  source 1..25: mol_type = other RNA
tccgacctgagctttgttgtagact

SEQ ID NO: 468 - RNA, 25 nt, synthetic construct
  misc_feature 1..25: note = oligonucleotide
  source 1..25: mol_type = other RNA
ccgacctgagctttgttgtagacta

SEQ ID NO: 469 - RNA, 20 nt, synthetic construct
  misc_feature 1..20: note = oligonucleotide
  source 1..20: mol_type = other RNA
cgacctgagctttgttgtag

SEQ ID NO: 470 - RNA, 25 nt, synthetic construct
  misc_feature 1..25: note = oligonucleotide
  source 1..25: mol_type = other RNA
cgacctgagctttgttgtagactat

SEQ ID NO: 471 - RNA, 25 nt, synthetic construct
  misc_feature 1..25: note = oligonucleotide
  source 1..25: mol_type = other RNA
gacctgagctttgttgtagactatc

SEQ ID NO: 472 - RNA, 25 nt, synthetic construct
  misc_feature 1..25: note = oligonucleotide
  source 1..25: mol_type = other RNA
acctgagctttgttgtagactatca

SEQ ID NO: 473 - RNA, 23 nt, synthetic construct
  misc_feature 1..23: note = oligonucleotide
  source 1..23: mol_type = other RNA
cctgagctttgttgtagactatc

SEQ ID NO: 474 - RNA, 20 nt, synthetic construct
  misc_feature 1..20: note = oligonucleotide
  source 1..20: mol_type = other RNA
catttttgacctacatgtgg

SEQ ID NO: 475 - RNA, 20 nt, synthetic construct
  misc_feature 1..20: note = oligonucleotide
  source 1..20: mol_type = other RNA
tttgacctacatgtggaaag

SEQ ID NO: 476 - RNA, 26 nt, synthetic construct
  misc_feature 1..26: note = oligonucleotide
  source 1..26: mol_type = other RNA
tacatttttgacctacatgtggaaag

SEQ ID NO: 477 - RNA, 17 nt, synthetic construct
  misc_feature 1..17: note = oligonucleotide
  source 1..17: mol_type = other RNA
ggtctccttacctatga

SEQ ID NO: 478 - RNA, 24 nt, synthetic construct
  misc_feature 1..24: note = oligonucleotide
  source 1..24: mol_type = other RNA
tcttacctatgactatggatgaga

SEQ ID NO: 479 - RNA, 22 nt, synthetic construct
  misc_feature 1..22: note = oligonucleotide
  source 1..22: mol_type = other RNA
atttttgacctacatgggaaag

SEQ ID NO: 480 - RNA, 23 nt, synthetic construct
  misc_feature 1..23: note = oligonucleotide
  source 1..23: mol_type = other RNA
tacgagttgattgtcggacccag

SEQ ID NO: 481 - RNA, 25 nt, synthetic construct
  misc_feature 1..25: note = oligonucleotide
  source 1..25: mol_type = other RNA
gtggtctccttacctatgactgtgg

SEQ ID NO: 482 - RNA, 24 nt, synthetic construct
  misc_feature 1..24: note = oligonucleotide
  source 1..24: mol_type = other RNA
tgtctcagtaatcttcttacctat

SEQ ID NO: 483 - RNA, 23 nt, synthetic construct
  misc_feature 1..23: note = oligonucleotide
  source 1..23: mol_type = other RNA
tgcatgttccagtcgttgtgtgg

SEQ ID NO: 484 - RNA, 25 nt, synthetic construct
  misc_feature 1..25: note = oligonucleotide
  source 1..25: mol_type = other RNA
cactattccagtcaaataggtctgg

SEQ ID NO: 485 - RNA, 25 nt, synthetic construct
  misc_feature 1..25: note = oligonucleotide
  source 1..25: mol_type = other RNA
atttaccaaccttcaggatcgagta

SEQ ID NO: 486 - RNA, 21 nt, synthetic construct
  misc_feature 1..21: note = oligonucleotide
  source 1..21: mol_type = other RNA
ggcctaaaacacatacacata

SEQ ID NO: 487 - RNA, 24 nt, synthetic construct
  misc_feature 1..24: note = oligonucleotide
  source 1..24: mol_type = other RNA
gataggtggtatcaacatctgtaa

SEQ ID NO: 488 - RNA, 21 nt, synthetic construct
  misc_feature 1..21: note = oligonucleotide
  source 1..21: mol_type = other RNA
gataggtggtatcaacatctg

SEQ ID NO: 489 - RNA, 19 nt, synthetic construct
  misc_feature 1..19: note = oligonucleotide
  source 1..19: mol_type = other RNA
cttcctggatggcttgaat

SEQ ID NO: 490 - RNA, 20 nt, synthetic construct
  misc_feature 1..20: note = oligonucleotide
  source 1..20: mol_type = other RNA
tgttgttgtttatgctcatt

SEQ ID NO: 491 - RNA, 19 nt, synthetic construct
  misc_feature 1..19: note = oligonucleotide
  source 1..19: mol_type = other RNA
gtacattaagatggacttc

SEQ ID NO: 492 - RNA, 20 nt, synthetic construct
  misc_feature 1..20: note = oligonucleotide
  source 1..20: mol_type = other RNA
ctgttgcagtaatctatgag

SEQ ID NO: 493 - RNA, 20 nt, synthetic construct
  misc_feature 1..20: note = oligonucleotide
  source 1..20: mol_type = other RNA
tgcagtaatctatgagtttc

SEQ ID NO: 494 - RNA, 18 nt, synthetic construct
  misc_feature 1..18: note = oligonucleotide
  source 1..18: mol_type = other RNA
gagtcttctaggagcctt

SEQ ID NO: 495 - RNA, 23 nt, synthetic construct
  misc_feature 1..23: note = oligonucleotide
  source 1..23: mol_type = other RNA
tgccattgtttcatcagctcttt

SEQ ID NO: 496 - RNA, 20 nt, synthetic construct
  misc_feature 1..20: note = oligonucleotide
  source 1..20: mol_type = other RNA
tcctgtaggacattggcagt

SEQ ID NO: 497 - RNA, 20 nt, synthetic construct
  misc_feature 1..20: note = oligonucleotide
  source 1..20: mol_type = other RNA
cttggagtcttctaggagcc

SEQ ID NO: 498 - RNA, 30 nt, synthetic construct
  misc_feature 1..30: note = oligonucleotide
  source 1..30: mol_type = other RNA
ccattttgtgaatgttttcttttgaacatc

SEQ ID NO: 499 - RNA, 24 nt, synthetic construct
  misc_feature 1..24: note = oligonucleotide
  source 1..24: mol_type = other RNA
cccattttgtgaatgttttctttt

SEQ ID NO: 500 - RNA, 24 nt, synthetic construct
  misc_feature 1..24: note = oligonucleotide
  source 1..24: mol_type = other RNA
gaaaattgtgcatttacccatttt

SEQ ID NO: 501 - RNA, 22 nt, synthetic construct
  misc_feature 1..22: note = oligonucleotide
  source 1..22: mol_type = other RNA
ttgtgcatttacccattttgtg

SEQ ID NO: 502 - RNA, 24 nt, synthetic construct
  misc_feature 1..24: note = oligonucleotide
  source 1..24: mol_type = other RNA
ccctgaggcattcccatcttgaat

SEQ ID NO: 503 - RNA, 20 nt, synthetic construct
  misc_feature 1..20: note = oligonucleotide
  source 1..20: mol_type = other RNA
aggacttacttgctttgttt

SEQ ID NO: 504 - RNA, 23 nt, synthetic construct
  misc_feature 1..23: note = oligonucleotide
  source 1..23: mol_type = other RNA
cttgaatttaggagattcatctg

SEQ ID NO: 505 - RNA, 23 nt, synthetic construct
  misc_feature 1..23: note = oligonucleotide
  source 1..23: mol_type = other RNA
catcttctgataattttcctgtt

SEQ ID NO: 506 - RNA, 20 nt, synthetic construct
  misc_feature 1..20: note = oligonucleotide
  source 1..20: mol_type = other RNA
ccattacagttgtctgtgtt

SEQ ID NO: 507 - RNA, 23 nt, synthetic construct
  misc_feature 1..23: note = oligonucleotide
  source 1..23: mol_type = other RNA
tgacagcctgtgaaatctgtgag

SEQ ID NO: 508 - RNA, 20 nt, synthetic construct
  misc_feature 1..20: note = oligonucleotide
  source 1..20: mol_type = other RNA
taatctgcctcttcttttgg

SEQ ID NO: 509 - RNA, 20 nt, synthetic construct
  misc_feature 1..20: note = oligonucleotide
  source 1..20: mol_type = other RNA
cagcagtagttgtcatctgc

SEQ ID NO: 510 - RNA, 27 nt, synthetic construct
  misc_feature 1..27: note = oligonucleotide
  source 1..27: mol_type = other RNA
gcctgagctgatctgctggcatcttgc

SEQ ID NO: 511 - RNA, 31 nt, synthetic construct
  misc_feature 1..31: note = oligonucleotide
  source 1..31: mol_type = other RNA
gcctgagctgatctgctggcatcttgcagtt

SEQ ID NO: 512 - RNA, 16 nt, synthetic construct
  misc_feature 1..16: note = oligonucleotide
  source 1..16: mol_type = other RNA
tctgctggcatcttgc

SEQ ID NO: 513 - RNA, 22 nt, synthetic construct
  misc_feature 1..22: note = oligonucleotide
  source 1..22: mol_type = other RNA
gccggttgacttcatcctgtgc

SEQ ID NO: 514 - RNA, 23 nt, synthetic construct
  misc_feature 1..23: note = oligonucleotide
  source 1..23: mol_type = other RNA
gtctgcatccaggaacatgggtc

SEQ ID NO: 515 - RNA, 24 nt, synthetic construct
  misc_feature 1..24: note = oligonucleotide
  source 1..24: mol_type = other RNA
tacttactgtctgtagctctttct

SEQ ID NO: 516 - RNA, 22 nt, synthetic construct
  misc_feature 1..22: note = oligonucleotide
  source 1..22: mol_type = other RNA
ctgcatccaggaacatgggtcc

SEQ ID NO: 517 - RNA, 24 nt, synthetic construct
  misc_feature 1..24: note = oligonucleotide
  source 1..24: mol_type = other RNA
gttgaagatctgatagccggttga

SEQ ID NO: 518 - RNA, 17 nt, synthetic construct
  misc_feature 1..17: note = oligonucleotide
  source 1..17: mol_type = other RNA
taggtgcctgccggctt

SEQ ID NO: 519 - RNA, 18 nt, synthetic construct
  misc_feature 1..18: note = oligonucleotide
  source 1..18: mol_type = other RNA
ttcagctgtagccacacc

SEQ ID NO: 520 - RNA, 21 nt, synthetic construct
  misc_feature 1..21: note = oligonucleotide
  source 1..21: mol_type = other RNA
ctgaactgctggaaagtcgcc

SEQ ID NO: 521 - RNA, 30 nt, synthetic construct
  misc_feature 1..30: note = oligonucleotide
  source 1..30: mol_type = other RNA
ctggcttccaaatgggacctgaaaaagaac

SEQ ID NO: 522 - RNA, 21 nt, synthetic construct
  misc_feature 1..21: note = oligonucleotide
  source 1..21: mol_type = other RNA
caatttttcccactcagtatt

SEQ ID NO: 523 - RNA, 19 nt, synthetic construct
  misc_feature 1..19: note = oligonucleotide
  source 1..19: mol_type = other RNA
ttgaagttcctggagtctt

SEQ ID NO: 524 - RNA, 22 nt, synthetic construct
  misc_feature 1..22: note = oligonucleotide
  source 1..22: mol_type = other RNA
tcctcaggaggcagctctaaat

SEQ ID NO: 525 - RNA, 16 nt, synthetic construct
  misc_feature 1..16: note = oligonucleotide
  source 1..16: mol_type = other RNA
tggctctctcccaggg

SEQ ID NO: 526 - RNA, 27 nt, synthetic construct
  misc_feature 1..27: note = oligonucleotide
  source 1..27: mol_type = other RNA
gagatggctctctcccagggaccctgg

SEQ ID NO: 527 - RNA, 17 nt, synthetic construct
  misc_feature 1..17: note = oligonucleotide
  source 1..17: mol_type = other RNA
gggcactttgtttggcg

SEQ ID NO: 528 - RNA, 19 nt, synthetic construct
  misc_feature 1..19: note = oligonucleotide
  source 1..19: mol_type = other RNA
ggtcccagcaagttgtttg

SEQ ID NO: 529 - RNA, 25 nt, synthetic construct
  misc_feature 1..25: note = oligonucleotide
  source 1..25: mol_type = other RNA
tgggatggtcccagcaagttgtttg

SEQ ID NO: 530 - RNA, 21 nt, synthetic construct
  misc_feature 1..21: note = oligonucleotide
  source 1..21: mol_type = other RNA
gtagagctctgtcattttggg

SEQ ID NO: 531 - RNA, 25 nt, synthetic construct
  misc_feature 1..25: note = oligonucleotide
  source 1..25: mol_type = other RNA
gctcaagagatccactgcaaaaaac

SEQ ID NO: 532 - RNA, 26 nt, synthetic construct
  misc_feature 1..26: note = oligonucleotide
  source 1..26: mol_type = other RNA
gccatacgtacgtatcataaacattc

SEQ ID NO: 533 - RNA, 25 nt, synthetic construct
  misc_feature 1..25: note = oligonucleotide
  source 1..25: mol_type = other RNA
tctgcaggatatccatgggctggtc

SEQ ID NO: 534 - RNA, 27 nt, synthetic construct
  misc_feature 1..27: note = oligonucleotide
  source 1..27: mol_type = other RNA
gatcctccctgttcgtcccctattatg

SEQ ID NO: 535 - RNA, 24 nt, synthetic construct
  misc_feature 1..24: note = oligonucleotide
  source 1..24: mol_type = other RNA
tgctttagactcctgtacctgata

SEQ ID NO: 536 - RNA, 18 nt, synthetic construct
  misc_feature 1..18: note = oligonucleotide
  source 1..18: mol_type = other RNA
ggcggcctttgtgttgac

SEQ ID NO: 537 - RNA, 25 nt, synthetic construct
  misc_feature 1..25: note = oligonucleotide
  source 1..25: mol_type = other RNA
ggacaggcctttatgttcgtgctgc

SEQ ID NO: 538 - RNA, 19 nt, synthetic construct
  misc_feature 1..19: note = oligonucleotide
  source 1..19: mol_type = other RNA
cctttatgttcgtgctgct

SEQ ID NO: 539 - RNA, 20 nt, synthetic construct
  misc_feature 1..20: note = oligonucleotide
  source 1..20: mol_type = other RNA
tcaaggaagatggcatttct

SEQ ID NO: 540 - RNA, 20 nt, synthetic construct
  misc_feature 1..20: note = oligonucleotide
  modified_base 5: mod_base = i
  source 1..20: mol_type = other RNA
tcaangaagatggcatttct

SEQ ID NO: 541 - RNA, 20 nt, synthetic construct
  misc_feature 1..20: note = oligonucleotide
  modified_base 6: mod_base = i
  source 1..20: mol_type = other RNA
tcaagnaagatggcatttct

SEQ ID NO: 542 - RNA, 20 nt, synthetic construct
  misc_feature 1..20: note = oligonucleotide
  modified_base 9: mod_base = i
  source 1..20: mol_type = other RNA
tcaaggaanatggcatttct

SEQ ID NO: 543 - RNA, 20 nt, synthetic construct
  misc_feature 1..20: note = oligonucleotide
  modified_base 12: mod_base = i
  source 1..20: mol_type = other RNA
tcaaggaagatngcatttct

SEQ ID NO: 544 - RNA, 20 nt, synthetic construct
  misc_feature 1..20: note = oligonucleotide
  modified_base 13: mod_base = i
  source 1..20: mol_type = other RNA
tcaaggaagatgncatttct

SEQ ID NO: 545 - RNA, 20 nt, synthetic construct
  misc_feature 1..20: note = oligonucleotide
  modified_base 1: mod_base = i
  source 1..20: mol_type = other RNA
ncaaggaagatggcatttct

SEQ ID NO: 546 - RNA, 20 nt, synthetic construct
  misc_feature 1..20: note = oligonucleotide
  modified_base 11: mod_base = i
  source 1..20: mol_type = other RNA
tcaaggaaganggcatttct

SEQ ID NO: 547 - RNA, 20 nt, synthetic construct
  misc_feature 1..20: note = oligonucleotide
  modified_base 16: mod_base = i
  source 1..20: mol_type = other RNA
tcaaggaagatggcanttct

SEQ ID NO: 548 - RNA, 20 nt, synthetic construct
  misc_feature 1..20: note = oligonucleotide
  modified_base 17: mod_base = i
  source 1..20: mol_type = other RNA
tcaaggaagatggcatntct

SEQ ID NO: 549 - RNA, 20 nt, synthetic construct
  misc_feature 1..20: note = oligonucleotide
  modified_base 18: mod_base = i
  source 1..20: mol_type = other RNA
tcaaggaagatggcattnct

SEQ ID NO: 550 - RNA, 20 nt, synthetic construct
  misc_feature 1..20: note = oligonucleotide
  modified_base 20: mod_base = i
  source 1..20: mol_type = other RNA
tcaaggaagatggcatttcn

SEQ ID NO: 551 - RNA, 20 nt, synthetic construct
  misc_feature 1..20: note = oligonucleotide
  modified_base 3: mod_base = i
  source 1..20: mol_type = other RNA
tcnaggaagatggcatttct

SEQ ID NO: 552 - RNA, 20 nt, synthetic construct
  misc_feature 1..20: note = oligonucleotide
  modified_base 4: mod_base = i
  source 1..20: mol_type = other RNA
tcanggaagatggcatttct

SEQ ID NO: 553 - RNA, 20 nt, synthetic construct
  misc_feature 1..20: note = oligonucleotide
  modified_base 7: mod_base = i
  source 1..20: mol_type = other RNA
tcaaggnagatggcatttct

SEQ ID NO: 554 - RNA, 20 nt, synthetic construct
  misc_feature 1..20: note = oligonucleotide
  modified_base 8: mod_base = i
  source 1..20: mol_type = other RNA
tcaaggangatggcatttct

SEQ ID NO: 555 - RNA, 20 nt, synthetic construct
  misc_feature 1..20: note = oligonucleotide
  modified_base 10: mod_base = i
  source 1..20: mol_type = other RNA
tcaaggaagntggcatttct

SEQ ID NO: 556 - RNA, 20 nt, synthetic construct
  misc_feature 1..20: note = oligonucleotide
  modified_base 15: mod_base = i
  source 1..20: mol_type = other RNA
tcaaggaagatggcntttct

SEQ ID NO: 557 - RNA, 25 nt, synthetic construct
  misc_feature 1..25: note = oligonucleotide
  modified_base 7: mod_base = i
  source 1..25: mol_type = other RNA
tttgccnctgcccaatgccatcctg

SEQ ID NO: 558 - RNA, 25 nt, synthetic construct
  misc_feature 1..25: note = oligonucleotide
  modified_base 10: mod_base = i
  source 1..25: mol_type = other RNA
tttgccgctncccaatgccatcctg

SEQ ID NO: 559 - RNA, 25 nt, synthetic construct
  misc_feature 1..25: note = oligonucleotide
  modified_base 17: mod_base = i
  source 1..25: mol_type = other RNA
tttgccgctgcccaatnccatcctg

SEQ ID NO: 560 - RNA, 25 nt, synthetic construct
  misc_feature 1..25: note = oligonucleotide
  modified_base 4: mod_base = i
  source 1..25: mol_type = other RNA
tttnccgctgcccaatgccatcctg

SEQ ID NO: 561 - RNA, 25 nt, synthetic construct
  misc_feature 1..25: note = oligonucleotide
  modified_base 25: mod_base = i
  source 1..25: mol_type = other RNA
tttgccgctgcccaatgccatcctn

SEQ ID NO: 562 - RNA, 25 nt, synthetic construct
  misc_feature 1..25: note = oligonucleotide
  modified_base 1: mod_base = i
  source 1..25: mol_type = other RNA
nttgccgctgcccaatgccatcctg

SEQ ID NO: 563 - RNA, 25 nt, synthetic construct
  misc_feature 1..25: note = oligonucleotide
  modified_base 2: mod_base = i
  source 1..25: mol_type = other RNA
tntgccgctgcccaatgccatcctg

SEQ ID NO: 564 - RNA, 25 nt, synthetic construct
  misc_feature 1..25: note = oligonucleotide
  modified_base 3: mod_base = i
  source 1..25: mol_type = other RNA
ttngccgctgcccaatgccatcctg

SEQ ID NO: 565 - RNA, 25 nt, synthetic construct
  misc_feature 1..25: note = oligonucleotide
  modified_base 9: mod_base = i
  source 1..25: mol_type = other RNA
tttgccgcngcccaatgccatcctg

SEQ ID NO: 566 - RNA, 25 nt, synthetic construct
  misc_feature 1..25: note = oligonucleotide
  modified_base 15: mod_base = i
  source 1..25: mol_type = other RNA
tttgccgctgcccantgccatcctg

SEQ ID NO: 567 - RNA, 25 nt, synthetic construct
  misc_feature 1..25: note = oligonucleotide
  modified_base 20: mod_base = i
  source 1..25: mol_type = other RNA
tttgccgctgcccaatgccntcctg

SEQ ID NO: 568 - RNA, 25 nt, synthetic construct
  misc_feature 1..25: note = oligonucleotide
  modified_base 4: mod_base = i
  modified_base 7: mod_base = i
  source 1..25: mol_type = other RNA
tttnccnctgcccaatgccatcctg

SEQ ID NO: 569 - RNA, 25 nt, synthetic construct
  misc_feature 1..25: note = oligonucleotide
  modified_base 16: mod_base = i
  source 1..25: mol_type = other RNA
tttgccgctgcccaangccatcctg

SEQ ID NO: 570 - RNA, 25 nt, synthetic construct
  misc_feature 1..25: note = oligonucleotide
  modified_base 21: mod_base = i
  source 1..25: mol_type = other RNA
tttgccgctgcccaatgccancctg

SEQ ID NO: 571 - RNA, 25 nt, synthetic construct
  misc_feature 1..25: note = oligonucleotide
  modified_base 24: mod_base = i
  source 1..25: mol_type = other RNA
tttgccgctgcccaatgccatccng

SEQ ID NO: 572 - RNA, 25 nt, synthetic construct
  misc_feature 1..25: note = oligonucleotide
  modified_base 14: mod_base = i
  source 1..25: mol_type = other RNA
tttgccgctgcccnatgccatcctg

SEQ ID NO: 573 - RNA, 20 nt, synthetic construct
  misc_feature 1..20: note = oligonucleotide
  modified_base 10: mod_base = i
  source 1..20: mol_type = other RNA
tcagcttctnttagccactg

SEQ ID NO: 574 - RNA, 20 nt, synthetic construct
  misc_feature 1..20: note = oligonucleotide
  modified_base 14: mod_base = i
  source 1..20: mol_type = other RNA
tcagcttctgttanccactg

SEQ ID NO: 575 - RNA, 20 nt, synthetic construct
  misc_feature 1..20: note = oligonucleotide
  modified_base 4: mod_base = i
  source 1..20: mol_type = other RNA
tcancttctgttagccactg

SEQ ID NO: 576 - RNA, 20 nt, synthetic construct
  misc_feature 1..20: note = oligonucleotide
  modified_base 20: mod_base = i
  source 1..20: mol_type = other RNA
tcagcttctgttagccactn

SEQ ID NO: 577 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

(19) United States
(12) Patent Application Publication
(10) Pub. No.: US 2024/0141339 A1
(43) Pub. Date: May 2, 2024

(54) Oligonucleotide Comprising Inosine for Treating DMD

(71) Applicant: BioMarin Technologies B.V., Leiden (NL)

(72) Inventors: Judith Christina Theodora van Deutekom, Dordrecht (NL); Josephus Johannes de Kimpe, Utrecht (NL); Gerard Johannes Platenburg, Voorschoten (NL)

(21) Appl. No.: 18/178,841

(22) Filed: Mar. 6, 2023

Related U.S. Application Data
(63) Continuation of application No. 17/302,500, filed on May 4, 2021, now Pat. No. 11,634,714.
Application No. 17/302,500 is a continuation of application No. 16/694,980, filed on Nov. 25, 2019, now Pat. No. 11,034,956.
Application No. 16/694,980 is a continuation of application No. 15/468,239, filed on Mar. 24, 2017, now Pat. No. 10,533,171.
Application No. 15/468,239 is a continuation of application No. 15/268,662, filed on Sep. 19, 2016, now abandoned.
Application No. 15/268,662 is a continuation of application No. 14/678,517, filed on Apr. 3, 2015, now abandoned.
Application No. 14/678,517 is a continuation of application No. 13/266,110, filed on Oct. 24, 2011, now abandoned.
(60) Provisional application No. 61/172,506, filed on Apr. 24, 2009.

(30) Foreign Application Priority Data
Apr. 24, 2009 (EP) 09158731.1

(51) Int. Cl. C12N 15/113; A61K 48/00; C12N 15/11

(52) U.S. Cl. CPC C12N 15/113; A61K 48/00; C12N 15/111; C12N 2310/11; C12N 2310/315; C12N 2310/3181; C12N 2310/321; C12N 2310/3231; C12N 2310/331; C12N 2310/333; C12N 2310/336; C12N 2320/33

(57) ABSTRACT

The invention provides an oligonucleotide comprising an inosine, and/or a nucleotide containing a base able to form a wobble base pair or a functional equivalent thereof, wherein the oligonucleotide, or a functional equivalent thereof, comprises a sequence which is complementary to at least part of a dystrophin pre-m RNA exon or at least part of a non-exon region of a dystrophin pre-m RNA said part being a contiguous stretch comprising at least 8 nucleotides. The invention further provides the use of said oligonucleotide for preventing or treating DMD or BMD.

### RELATED APPLICATIONS

This application is a continuation of U.S. application Ser. No. 17/302,500, filed May 4, 2021, which is a continuation of U.S. application Ser. No. 16/694,980, filed Nov. 25, 2019, now U.S. Pat. No. 11,034,956, which is a continuation of U.S. application Ser. No. 15/468,239, filed Mar. 24, 2017, now U.S. Pat. No. 10,533,171, which is a continuation of U.S. application Ser. No. 15/168,662, filed May 31, 2016, which is a continuation of U.S. application Ser. No. 14/678,517, filed Apr. 3, 2015, which is a continuation of U.S. application Ser. No. 13/266,110, filed Oct. 24, 2011, which is a 371 of PCT International Application No. PCT/NL2010/050230, filed Apr. 26, 2010, which claims priority to European application No. 09158731.1, filed Apr. 24, 2009 and U.S. provisional application No. 61/172,506, filed Apr. 24, 2009, the entire contents of each of which are incorporated herein by reference in their entirety.

### SEQUENCE LISTING

This application contains a Sequence Listing, which is being submitted herewith as an XML file named “0105 09 USCN6.xml”, created on Jul. 6, 2023, size 748,331 bytes, which is incorporated by reference herein in its entirety.

### FIELD OF THE INVENTION

The invention relates to the fields of molecular biology and medicine.

### BACKGROUND OF THE INVENTION

A muscle disorder is a disease that usually has a significant impact on the life of an individual. A muscle disorder can have either a genetic cause or a non-genetic cause. An important group of muscle diseases with a genetic cause are Becker Muscular Dystrophy (BMD) and Duchenne Muscular Dystrophy (DMD). These disorders are caused by defects in a gene for a muscle protein.

Becker Muscular Dystrophy and Duchenne Muscular Dystrophy are genetic muscular dystrophies with a relatively high incidence. In both Duchenne and Becker muscular dystrophy the muscle protein dystrophin is affected. In Duchenne dystrophin is absent, whereas in Becker some dystrophin is present but its production is most often not sufficient and/or the dystrophin present is abnormally formed. Both diseases are associated with recessive X-linked inheritance. DMD results from a frameshift mutation in the DMD gene. The frameshift in the DMD gene's transcript (mRNA) results in the production of a truncated non-functional dystrophin protein, resulting in progressive muscle wasting and weakness. BMD occurs as a mutation does not cause a frame-shift in the DMD transcript (mRNA). As in BMD some partly to largely functional dystrophin is present in contrast to DMD where dystrophin is absent, BMD has generally less severe symptoms then DMD. The onset of DMD is earlier than BMD. DMD usually manifests itself in early childhood, BMD in the teens or in early adulthood. The progression of BMD is slower and less predictable than DMD. Patients with BMD can survive into mid to late adulthood. Patients with DMD rarely survive beyond their thirties.

Dystrophin plays an important structural role in the muscle fiber, connecting the extracellular matrix and the cytoskeleton. The N-terminal region binds actin, whereas the C-terminal end is part of the dystrophin glycoprotein complex (DGC), which spans the sarcolemma. In the absence of dystrophin, mechanical stress leads to sarcolemmal ruptures, causing an uncontrolled influx of calcium into the muscle fiber interior, thereby triggering calcium-activated proteases and fiber necrosis.

For most genetic muscular dystrophies no clinically applicable and effective therapies are currently available. Exon skipping techniques are nowadays explored in order to combat genetic muscular dystrophies. Promising results have recently been reported by us and others on a genetic therapy aimed at restoring the reading frame of the dystrophin pre-mRNA in cells from the mdx mouse, the GRMD dog (reference 59) and DMD patients¹⁻¹¹. By the targeted skipping of a specific exon, a DMD phenotype (lacking dystrophin) is converted into a milder BMD phenotype (partly to largely functional dystrophin). The skipping of an exon is preferably induced by the binding of antisense oligoribonucleotides (AONs) targeting either one or both of the splice sites, or exon-internal sequences. Since an exon will only be included in the mRNA when both the splice sites are recognised by the spliceosome complex, splice sites have been considered obvious targets for AONs. More preferably, one or more AONs are used which are specific for at least part of one or more exonic sequences involved in correct splicing of the exon. Using exon-internal AONs specific for an exon 46 sequence, we were previously able to modulate the splicing pattern in cultured myotubes from two different DMD patients with an exon 45 deletion¹¹. Following AON treatment, exon 46 was skipped, which resulted in a restored reading frame and the induction of dystrophin synthesis in at least 75% of the cells. We have recently shown that exon skipping can also efficiently be induced in human control and patient muscle cells for 39 different DMD exons using exon-internal AONs^{1, 2, 11-15}.

Hence, exon skipping techniques applied on the dystrophin gene result in the generation of at least partially functional—albeit shorter—dystrophin protein in DMD patients. Since DMD is caused by a dysfunctional dystrophin protein, it would be expected that the symptoms of DMD are sufficiently alleviated once a DMD patient has been provided with functional dystrophin protein. However, the present invention provides the insight that, even though exon skipping techniques are capable of inducing dystrophin synthesis, the oligonucleotide used for exon skipping technique can be improved any further by incorporating an inosine and/or a nucleotide containing a base able to form a wobble base pair in said oligonucleotide.

### DESCRIPTION OF THE INVENTION

### Oligonucleotide

In a first aspect, there is provided an oligonucleotide comprising an inosine and/or a nucleotide containing a base able to form a wobble base pair or a functional equivalent thereof, wherein the oligonucleotide, or a functional equivalent thereof, comprises a sequence which is complementary to at least part of a dystrophin pre-mRNA exon or at least part of a non-exon region of a dystrophin pre-mRNA said part being a contiguous stretch comprising at least 8 nucleotides.

The use of an inosine and/or a nucleotide containing a base able to form a wobble base pair in an oligonucleotide of the invention is very attractive as explained below. Inosine for example is a known modified base which can pair with three bases: uracil, adenine, and cytosine. Inosine is a nucleoside that is formed when hypoxanthine is attached to a ribose ring (also known as a ribofuranose) via a ß-N9-glycosidic bond. Inosine is commonly found in tRNAs and is essential for proper translation of the genetic code in wobble base pairs. A wobble base pair is a G-U and I-U/I-A/I-C pair fundamental in RNA secondary structure. Its thermodynamic stability is comparable to that of the Watson-Crick base pair. Wobble base pairs are critical for the proper translation of the genetic code. The genetic code makes up for disparities in the number of amino acids (20) for triplet codons (64), by using modified base pairs in the first base of the anti-codon. Similarly, when designing primers for polymerase chain reaction, inosine is useful in that it will indiscriminately pair with adenine, thymine, or cytosine. A first advantage of using such a base allows one to design a primer that spans a single nucleotide polymorphism (SNP), without worry that the polymorphism will disrupt the primer's annealing efficiency. Therefore in the invention, the use of such a base allows to design an oligonucleotide that may be used for an individual having a SNP within the dystrophin pre-mRNA stretch which is targeted by an oligonucleotide of the invention. A second advantage of using an inosine and/or a base able to form a wobble base pair in an oligonucleotide of the invention is when said oligonucleotide would normally contain a CpG if one would have designed it as being complementary to at least part of a dystrophin pre-mRNA exon or at least part of a non-exon region of a dystrophin pre-mRNA said part being a contiguous stretch comprising at least 8 nucleotides. The presence of a CpG in an oligonucleotide is usually associated with an increased immunogenicity of said oligonucleotide (reference 60). This increased immunogenicity is undesired since it may induce the breakdown of muscle fibers. Replacing one, two or more CpG by the corresponding inosine and/or a base able to form a wobble base pair in said oligonucleotide is expected to provide an oligonucleotide with a decreased and/or acceptable level of immunogenicity. Immunogenicity may be assessed in an animal model by assessing the presence of CD4⁺ and/or CD8⁺ cells and/or inflammatory mononucleocyte infiltration in muscle biopsy of said animal.

Immunogenicity may also be assessed in blood of an animal or of a human being treated with an oligonucleotide of the invention by detecting the presence of a neutralizing antibody and/or an antibody recognizing said oligonucleotide using a standard immunoassay known to the skilled person.

An increase in immunogenicity preferably corresponds to a detectable increase of at least one of these cell types by comparison to the amount of each cell type in a corresponding muscle biopsy of an animal before treatment or treated with a corresponding oligonucleotide having at least one inosine and/or a base able to form a wobble base pair. Alternatively, an increase in immunogenicity may be assessed by detecting the presence or an increasing amount of a neutralizing antibody or an antibody recognizing said oligonucleotide using a standard immunoassay.

A decrease in immunogenicity preferably corresponds to a detectable decrease of at least one of these cell types by comparison to the amount of corresponding cell type in a corresponding muscle biopsy of an animal before treatment or treated with a corresponding oligonucleotide having no inosine and/or a base able to form a wobble base pair. Alternatively a decrease in immunogenicity may be assessed by the absence of or a decreasing amount of said compound and/or neutralizing antibodies using a standard immunoassay.

A third advantage of using an inosine and/or a base able to form a wobble base pair in an oligonucleotide of the invention is to avoid or decrease a potential multimerisation or aggregation of oligonucleotides. It is for example known that an oligonucleotide comprising a G-quartet motif has the tendency to form a quadruplex, a multimer or aggregate formed by the Hoogsteen base-pairing of four single-stranded oligonucleotides (reference 61), which is of course not desired: as a result the efficiency of the oligonucleotide is expected to be decreased. Multimerisation or aggregation is preferably assessed by standard polyacrylamid non-denaturing gel electrophoresis techniques known to the skilled person. In a preferred embodiment, less than 20% or 15%, 10%, 7%, 5% or less of a total amount of an oligonucleotide of the invention has the capacity to multimerise or aggregate assessed using the assay mentioned above.

A fourth advantage of using an inosine and/or a base able to form a wobble base pair in an oligonucleotide of the invention is thus also to avoid quadruplex structures which have been associated with antithrombotic activity (reference 62) as well as with the binding to, and inhibition of, the macrophage scavenger receptor (reference 63).

A fifth advantage of using an inosine and/or a base able to form a wobble base pair in an oligonucleotide of the invention is to allow to design an oligonucleotide with improved RNA binding kinetics and/or thermodynamic properties. The RNA binding kinetics and/or thermodynamic properties are at least in part determined by the melting temperature of an oligonucleotide (Tm; calculated with the oligonucleotide properties calculator (http://www.unc.edu/^{˜}cail/biotool/oligo/index.html) for single stranded RNA using the basic Tm and the nearest neighbour model), and/or the free energy of the AON-target exon complex (using RNA structure version 4.5). If a Tm is too high, the oligonucleotide is expected to be less specific. An acceptable Tm and free energy depend on the sequence of the oligonucleotide. Therefore, it is difficult to give preferred ranges for each of these parameters. An acceptable Tm may be ranged between 35 and 65° C. and an acceptable free energy may be ranged between 15 and 45 kcal/mol.

The skilled person may therefore first choose an oligonucleotide as a potential therapeutic compound. In a second step, he may use the invention to further optimise said oligonucleotide by decreasing its immunogenicity and/or avoiding aggregation and/or quadruplex formation and/or by optimizing its Tm and/or free energy of the AON-target complex. He may try to introduce at least one inosine and/or a base able to form a wobble base pair in said oligonucleotide at a suitable position and assess how the immunogenicity and/or aggregation and/or quadruplex formation and/or Tm and/or free energy of the AON-target complex have been altered by the presence of said inosine and/or a base able to form a wobble base pair. If the alteration does not provide the desired alteration or decrease of immunogenicity and/or aggregation and/or quadruplex formation and/or its Tm and/or free energy of the AON-target complex he may choose to introduce a further inosine and/or a base able to form a wobble base pair in said oligonucleotide and/or to introduce a given inosine and/or a base able to form a wobble base pair at a distinct suitable position within said oligonucleotide.

An oligonucleotide comprising an inosine and/or a base able to form a wobble base pair may be defined as an oligonucleotide wherein at least one nucleotide has been substituted with an inosine and/or a base able to form a wobble base pair. The skilled person knows how to test whether a nucleotide contains a base able to form a wobble base pair. Since for example inosine can form a base pair with uracil, adenine, and/or cytosine, it means that at least one nucleotide able to form a base pair with uracil, adenine and/or cytosine has been substituted with inosine. However, in order to safeguard specificity, the inosine containing oligonucleotide preferably comprises the substitution of at least one, two, three, four nucleotide(s) able to form a base pair with uracil or adenine or cytosine as long as an acceptable level of a functional activity of said oligonucleotide is retained as defined later herein.

An oligonucleotide comprising an inosine and/or a base able to form a wobble base pair is preferably an olignucleotide, which is still able to exhibit an acceptable level of a functional activity of a corresponding oligonucleotide not comprising an inosine and/or a base able to form a wobble base pair. A functional activity of said oligonucleotide is preferably to provide an individual with a functional dystrophin protein anchor mRNA and/or at least in part decreasing the production of an aberrant dystrophin protein anchor mRNA. Each of these features are later defined herein. An acceptable level of such a functional activity is preferably at least 50%, 60%, 70%, 80%, 90%, 95% or 100% of the functional activity of the corresponding oligonucleotide which does not comprise an inosine and/or a base able to form a wobble base pair. Such functional activity may be as measured in a muscular tissue or in a muscular cell of an individual or in vitro in a cell by comparison to the functional activity of a corresponding oligonucleotides not comprising an inosine and/or a base able to form a wobble base pair. The assessment of the functionality may be carried out at the mRNA level, preferably using RT-PCR. The assessment of the functionality may be carried out at the protein level, preferably using western blot analysis or immunofluorescence analysis of cross-sections.

Within the context of the invention, an inosine and/or a base able to form a wobble base pair as present in an oligonucleotide is/are present in a part of said oligonucleotide which is complementary to at least part of a dystrophin pre-mRNA exon or at least part of a non-exon region of a dystrophin pre-mRNA said part being a contiguous stretch comprising at least 8 nucleotides. Therefore, in a preferred embodiment, an oligonucleotide comprising an inosine and/or a nucleotide containing a base able to form a wobble base pair or a functional equivalent thereof, wherein the oligonucleotide, or a functional equivalent thereof, comprises a sequence which is complementary to at least part of a dystrophin pre-mRNA exon or at least part of a non-exon region of a dystrophin pre-mRNA said part being a contiguous stretch comprising at least 8 nucleotides and wherein said inosine and/or a nucleotide containing a base able is/are present within the oligonucleotide sequence which is complementary to at least part of a dystrophin pre-mRNA as defined in previous sentence.

However, as later defined herein such inosine and/or a base able to form a wobble base pair may also be present in a linking moiety present in an oligonucleotide of the invention. Preferred linking moieties are later defined herein.

In a preferred embodiment, such oligonucleotide is preferably a medicament. More preferably, said medicament is for preventing or treating Duchenne Muscular Dystrophy or Becker Muscular Dystrophy in an individual or a patient. As defined herein a DMD pre-mRNA preferably means the pre-mRNA of a DMD gene of a DMD or BMD patient. A patient is preferably intended to mean a patient having DMD or BMD or a patient susceptible to develop DMD or BMD due to his or her genetic background. In the case of a DMD patient, an oligonucleotide used will preferably correct at least one of the DMD mutations as present in the DMD gene of said patient and therefore will preferably create a dystrophin that will look like a BMD dystrophin: said dystrophin will preferably be a functional dystrophin as later defined herein.

In the case of a BMD patient, an oligonucleotide as used will preferably correct at least one of the BMD mutations as present in the DMD gene of said patient and therefore will preferably create a, or more of a, dystrophin, which will be more functional than the dystrophin which was originally present in said BMD patient. Even more preferably, said medicament provides an individual with a functional or more (of a) functional dystrophin protein and/or mRNA and/or at least in part decreases the production of an aberrant dystrophin protein and/or mRNA.

Preferably, a method of the invention by inducing and/or promoting skipping of at least one exon of the DMD pre-mRNA as identified herein in one or more cells, preferably muscle cells of a patient, provides said patient with an increased production of a more (of a) functional dystrophin protein and/or mRNA and/or decreases the production of an aberrant or less functional dystrophin protein and/or mRNA in said patient.

Providing a patient with a more functional dystrophin protein and/or mRNA and/or decreasing the production of an aberrant dystrophin protein and/or mRNA in said patient is typically applied in a DMD patient. Increasing the production of a more functional or functional dystrophin and/or mRNA is typically applied in a BMD patient.

Therefore a preferred method is a method, wherein a patient or one or more cells of said patient is provided with an increased production of a more functional or functional dystrophin protein and/or mRNA and/or wherein the production of an aberrant dystrophin protein and/or mRNA in said patient is decreased, wherein the level of said aberrant or more functional dystrophin protein and/or mRNA is assessed by comparison to the level of said dystrophin and/or mRNA in said patient at the onset of the method.

As defined herein, a functional dystrophin is preferably a wild type dystrophin corresponding to a protein having the amino acid sequence as identified in SEQ ID NO: 1. A functional dystrophin is preferably a dystrophin, which has an actin binding domain in its N terminal part (first 240 amino acids at the N terminus), a cystein-rich domain (amino acid 3361 till 3685) and a C terminal domain (last 325 amino acids at the C terminus) each of these domains being present in a wild type dystrophin as known to the skilled person. The amino acids indicated herein correspond to amino acids of the wild type dystrophin being represented by SEQ ID NO: 1. In another embodiment, a functional dystrophin is a dystrophin, which exhibits at least to some extent an activity of a wild type dystrophin. “At least to some extent” preferably means at least 30%, 40%, 50%, 60%, 70%, 80%, 90%, 95% or 100% of a corresponding activity of a wild type functional dystrophin. In this context, an activity of a wild type dystrophin is preferably binding to actin and to the dystrophin-associated glycoprotein complex (DGC)⁵⁶. Binding of dystrophin to actin and to the DGC complex may be visualized by either co-immunoprecipitation using total protein extracts or immunofluorescence analysis of cross-sections, from a biopsy of a muscle suspected to be dystrophic, as known to the skilled person.

Individuals suffering from Duchenne muscular dystrophy typically have a mutation in the gene encoding dystrophin that prevents synthesis of the complete protein, i.e a premature stop prevents the synthesis of the C-terminus of the protein. In Becker muscular dystrophy the dystrophin gene also comprises a mutation compared to the wild type but the mutation does typically not include a premature stop and the C-terminus of the protein is typically synthesized. As a result a functional dystrophin protein is synthesized that has at least the same activity in kind as a wild type protein, although not necessarily the same amount of activity. In a preferred embodiment, a functional dystrophin protein means an in frame dystrophin gene. The genome of a BMD individual typically encodes a dystrophin protein comprising the N terminal part (first 240 amino acids at the N terminus), a cystein-rich domain (amino acid 3361 till 3685) and a C terminal domain (last 325 amino acids at the C terminus) but its central rod shaped domain may be shorter than the one of a wild type dystrophin⁵⁶. Exon-skipping for the treatment of DMD is preferably but not exclusively directed to overcome a premature stop in the pre-mRNA by skipping an exon in the rod-domain shaped domain to correct the reading frame and allow synthesis of remainder of the dystrophin protein including the C-terminus, albeit that the protein is somewhat smaller as a result of a smaller rod domain. In a preferred embodiment, an individual having DMD and being treated using an oligonucleotide as defined herein will be provided a dystrophin, which exhibits at least to some extent an activity of a wild type dystrophin. More preferably, if said individual is a Duchenne patient or is suspected to be a Duchenne patient, a functional dystrophin is a dystrophin of an individual having BMD: preferably said dystrophin is able to interact with both actin and the DGC, but its central rod shaped domain may be shorter than the one of a wild type dystrophin (Aartsma-Rus et al (2006, ref 56). The central rod domain of wild type dystrophin comprises 24 spectrin-like repeats⁵⁶. For example, a central rod shaped domain of a dystrophin as provided herein may comprise 5 to 23, 10 to 22 or 12 to 18 spectrin-like repeats as long as it can bind to actin and to DGC. Decreasing the production of an aberrant dystrophin in said patient or in a cell of said patient may be assessed at the mRNA level and preferably means that 99%, 90%, 80%, 70%, 60%, 50%, 40%, 30%, 20%, 10%, 5% or less of the initial amount of aberrant dystrophin mRNA, is still detectable by RT PCR. An aberrant dystrophin mRNA or protein is also referred to herein as a non-functional or less to non-functional or semi-functional dystrophin mRNA or protein. A non-functional pre-mRNA dystrophin is preferably leads to an out of frame dystrophin protein, which means that no dystrophin protein will be produced and/or detected. A non functional dystrophin protein is preferably a dystrophin protein which is not able to bind actin and/or members of the DGC protein complex. A non-functional dystrophin protein or dystrophin mRNA does typically not have, or does not encode a dystrophin protein with an intact C-terminus of the protein.

Increasing the production of a functional dystrophin in said patient or in a cell of said patient may be assessed at the mRNA level (by RT-PCR analysis) and preferably means that a detectable amount of a functional or in frame dystrophin mRNA is detectable by RT PCR. In another embodiment, 1%, 5%, 10%, 20%, 30%, 40%, 50%, 60%, 70%, 80%, 90% or more of the detectable dystrophin mRNA is a functional or in frame dystrophin mRNA.

Increasing the production of a functional dystrophin in said patient or in a cell of said patient may be assessed at the protein level (by immunofluorescence and western blot analyses) and preferably means that a detectable amount of a functional dystrophin protein is detectable by immunofluorescence or western blot analysis. In another embodiment, 1%, 5%, 10%, 20%, 30%, 40%, 50%, 60%, 70%, 80%, 90% or more of the detectable dystrophin protein is a functional dystrophin protein.

An increase or a decrease is preferably assessed in a muscular tissue or in a muscular cell of an individual or a patient by comparison to the amount present in said individual or patient before treatment with said molecule or composition of the invention. Alternatively, the comparison can be made with a muscular tissue or cell of said individual or patient, which has not yet been treated with said oligonucleotide or composition in case the treatment is local.

In a preferred method, one or more symptom(s) from a DMD or a BMD patient is/are alleviated and/or one or more characteristic(s) of a muscle cell or tissue from a DMD or a BMD patient is/are alleviated using a molecule or a composition of the invention. Such symptoms may be assessed on the patient self. Such characteristics may be assessed at the cellular, tissue level of a given patient. An alleviation of one or more characteristics may be assessed by any of the following assays on a myogenic cell or muscle cell from a patient: reduced calcium uptake by muscle cells, decreased collagen synthesis, altered morphology, altered lipid biosynthesis, decreased oxidative stress, and/or improved muscle fiber function, integrity, and/or survival. These parameters are usually assessed using immunofluorescence and/or histochemical analyses of cross sections of muscle biopsies.

Alleviating one or more symptom(s) of Duchenne Muscular Dystrophy or Becker Muscular Dystrophy in an individual using a molecule or a composition of the invention may be assessed by any of the following assays: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, improvement of the quality of life. Each of these assays is known to the skilled person. As an example, the publication of Manzur at al (2008, ref 58) gives an extensive explanation of each of these assays. For each of these assays, as soon as a detectable improvement or prolongation of a parameter measured in an assay has been found, it will preferably mean that one or more symptoms of Duchenne Muscular Dystrophy or Becker Muscular Dystrophy has been alleviated in an individual using a molecule or composition of the invention. Detectable improvement or prolongation is preferably a statistically significant improvement or prolongation as described in Hodgetts et al (2006, ref 57). Alternatively, the alleviation of one or more symptom(s) of Duchenne Muscular Dystrophy or Becker Muscular Dystrophy may be assessed by measuring an improvement of a muscle fiber function, integrity and/or survival as later defined herein.

An oligonucleotide as used herein preferably comprises an antisense oligonucleotide or antisense oligoribonucleotide. In a preferred embodiment an exon skipping technique is applied. Exon skipping interferes with the natural splicing processes occurring within a eukaryotic cell. In higher eukaryotes the genetic information for proteins in the DNA of the cell is encoded in exons which are separated from each other by intronic sequences. These introns are in some cases very long. The transcription machinery of eukaryotes generates a pre-mRNA which contains both exons and introns, while the splicing machinery, often already during the production of the pre-mRNA, generates the actual coding region for the protein by splicing together the exons present in the pre-mRNA.

Exon-skipping results in mature mRNA that lacks at least one skipped exon. Thus, when said exon codes for amino acids, exon skipping leads to the expression of an altered product. Technology for exon-skipping is currently directed towards the use of antisense oligonucleotides (AONs). Much of this work is done in the mdx mouse model for Duchenne muscular dystrophy. The mdx mouse carries a nonsense mutation in exon 23. Despite the mdx mutation, which should preclude the synthesis of a functional dystrophin protein, rare, naturally occurring dystrophin positive fibers have been observed in mdx muscle tissue. These dystrophin-positive fibers are thought to have arisen from an apparently naturally occurring exon-skipping mechanism, either due to somatic mutations or through alternative splicing. AONs directed to, respectively, the 3′ and/or 5′ splice sites of introns 22 and 23 in dystrophin pre-mRNA, have been shown to interfere with factors normally involved in removal of intron 23 so that also exon 23 was removed from the mRNA^{3, 5, 6, 89, 40}.

By the targeted skipping of a specific exon, a DMD phenotype is converted into a milder BMD phenotype. The skipping of an exon is preferably induced by the binding of AONs targeting either one or both of the splice sites, or exon-internal sequences. An oligonucleotide directed toward an exon internal sequence typically exhibits no overlap with non-exon sequences. It preferably does not overlap with the splice sites at least not insofar, as these are present in the intron. An oligonucleotide directed toward an exon internal sequence preferably does not contain a sequence complementary to an adjacent intron. Further provided is thus an oligonucleotide according to the invention, wherein said oligonucleotide, or a functional equivalent thereof, is for inhibiting inclusion of an exon of a dystrophin pre-mRNA into mRNA produced from splicing of said pre-mRNA. An exon skipping technique is preferably applied such that the absence of an exon from mRNA produced from dystrophin pre-mRNA generates a coding region for a more functional—albeit shorter—dystrophin protein. In this context, inhibiting inclusion of an exon preferably means that the detection of the original, aberrant dystrophin mRNA and/or protein is decreased as earlier defined herein.

Since an exon of a dystrophin pre-mRNA will only be included into the resulting mRNA when both the splice sites are recognised by the spliceosome complex, splice sites have been obvious targets for AONs. One embodiment therefore provides an oligonucleotide, or a functional equivalent thereof, comprising a sequence which is complementary to a non-exon region of a dystrophin pre mRNA. In one embodiment an AON is used which is solely complementary to a non-exon region of a dystrophin pre mRNA. This is however not necessary: it is also possible to use an AON which comprises an intron-specific sequence as well as exon-specific sequence. Such AON comprises a sequence which is complementary to a non-exon region of a dystrophin pre mRNA, as well as a sequence which is complementary to an exon region of a dystrophin pre mRNA. Of course, an AON is not necessarily complementary to the entire sequence of a dystrophin exon or intron. AONs, which are complementary to a part of such exon or intron are preferred. An AON is preferably complementary to at least part of a dystrophin exon and/or intron, said part having at least 8, 10, 13, 15, 20 nucleotides.

Splicing of a dystrophin pre-mRNA occurs via two sequential transesterification reactions. First, the 2′OH of a specific branch-point nucleotide within the intron that is defined during spliceosome assembly performs a nucleophilic attack on the first nucleotide of the intron at the 5′ splice site forming the lariat intermediate. Second, the 3′OH of the released 5′ exon then performs a nucleophilic attack at the last nucleotide of the intron at the 3′ splice site thus joining the exons and releasing the intron lariat. The branch point and splice sites of an intron are thus involved in a splicing event. Hence, an oligonucleotide comprising a sequence, which is complementary to such branch point and/or splice site is preferably used for exon skipping. Further provided is therefore an oligonucleotide, or a functional equivalent thereof, which comprises a sequence which is complementary to a splice site and/or branch point of a dystrophin pre mRNA.

Since splice sites contain consensus sequences, the use of an oligonucleotide or a functional equivalent thereof (herein also called an AON) comprising a sequence which is complementary of a splice site involves the risk of promiscuous hybridization. Hybridization of AONs to other splice sites than the sites of the exon to be skipped could easily interfere with the accuracy of the splicing process. To overcome these and other potential problems related to the use of AONs which are complementary to an intron sequence, one preferred embodiment provides an oligonucleotide, or a functional equivalent thereof, comprising a sequence which is complementary to a dystrophin pre-mRNA exon. Preferably, said AON is capable of specifically inhibiting an exon inclusion signal of at least one exon in said dystrophin pre-mRNA. Interfering with an exon inclusion signal (EIS) has the advantage that such elements are located within the exon. By providing an AON for the interior of the exon to be skipped, it is possible to interfere with the exon inclusion signal thereby effectively masking the exon from the splicing apparatus. The failure of the splicing apparatus to recognize the exon to be skipped thus leads to exclusion of the exon from the final mRNA. This embodiment does not interfere directly with the enzymatic process of the splicing machinery (the joining of the exons). It is thought that this allows the method to be more specific and/or reliable. It is thought that an EIS is a particular structure of an exon that allows splice acceptor and donor to assume a particular spatial conformation. In this concept, it is the particular spatial conformation that enables the splicing machinery to recognize the exon. However, the invention is certainly not limited to this model. In a preferred embodiment, use is made of an oligonucleotide, which is capable of binding to an exon and is capable of inhibiting an EIS. An AON may specifically contact said exon at any point and still be able to specifically inhibit said EIS.

Within the context of the invention, a functional equivalent of an oligonucleotide preferably means an oligonucleotide as defined herein wherein one or more nucleotides have been substituted and wherein an activity of said functional equivalent is retained to at least some extent. Preferably, an activity of said functional equivalent is providing a functional dystrophin protein. Said activity of said functional equivalent is therefore preferably assessed by quantifying the amount of a functional dystrophin protein or by quantifying the amount of a functional dystrophin mRNA. A functional dystrophin protein (or a functional dystrophin mRNA) is herein preferably defined as being a dystrophin protein (or a dystrophin protein encoded by said mRNA) able to bind actin and members of the DGC protein. The assessment of said activity of an oligonucleotide is preferably done by RT-PCR (m-RNA) or by immunofluorescence or Western blot analyses (protein). Said activity is preferably retained to at least some extent when it represents at least 50%, or at least 60%, or at least 70% or at least 80% or at least 90% or at least 95% or more of corresponding activity of said oligonucleotide the functional equivalent derives from. Such activity may be measured in a muscular tissue or in a muscular cell of an individual or in vitro in a cell by comparison to an activity of a corresponding oligonucleotide of said oligonucleotide the functional equivalent derives from. Throughout this application, when the word oligonucleotide is used it may be replaced by a functional equivalent thereof as defined herein.

Hence, an oligonucleotide, or a functional equivalent thereof, comprising or consisting of a sequence which is complementary to a dystrophin pre-mRNA exon provides good DMD therapeutic results. In one preferred embodiment an oligonucleotide, or a functional equivalent thereof, is used which comprises or consists of a sequence which is complementary to at least part of either dystrophin pre-mRNA exons 2 to 75 said part having or comprising at least 13 nucleotides. However, said part may also have at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides. A part of dystrophin pre-mRNA to which an oligonucleotide is complementary may also be called a contiguous stretch of dystrophin pre-mRNA.

Most preferably an AON is used which comprises or consists of a sequence which is complementary to at least part of dystrophin pre-mRNA exon 51, 45, 53, 44, 46, 52, 50, 43, 6, 7, 8, 55, 2, 11, 17, 19, 21, 57, 59, 62, 63, 65, 66, 69, and/or 75 said part having or comprising at least 13 nucleotides. However, said part may also have at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides. More preferred oligonucleotides are represented by a sequence that comprises or consists of each of the following sequences SEQ ID NO: 2 to SEQ ID NO:539 wherein at least one inosine and/or a base able to form a wobble base pair is present in said sequence. Preferably, an inosine has been introduced in one of these sequences to replace a guanosine, adenine, or a uracil. Accordingly, an even more preferred oligonucleotide as used herein is represented by a sequence that comprises or consists of SEQ ID NO:2 to SEQ ID NO:486 or SEQ ID NO:539, even more preferably SEQ ID NO:2 to NO 237 or SEQ ID NO:539, most preferably SEQ ID NO:76 wherein at least one inosine and/or a base able to form a wobble base pair is present in said sequence. Preferably, an inosine has been introduced in one of these sequences to replace a guanosine, adenine, or a uracil.

Accordingly, in another preferred embodiment, an oligonucleotide as used herein is represented by a sequence that comprises or consists of SEQ ID NO:540 to SEQ ID NO:576. More preferably, an oligonucleotide as used herein is represented by a sequence that comprises or consists of SEQ ID NO:557.

Said exons are listed in decreasing order of patient population applicability. Hence, the use of an AON comprising a sequence, which is complementary to at least part of dystrophin pre-mRNA exon 51 is suitable for use in a larger part of the DMD patient population as compared to an AON comprising a sequence which is complementary to dystrophin pre-mRNA exon 44, et cetera.

In a preferred embodiment, an oligonucleotide of the invention, which comprises a sequence that is complementary to part of dystrophin pre-mRNA is such that the complementary part is at least 50% of the length of the oligonucleotide of the invention, more preferably at least 60%, even more preferably at least 70%, even more preferably at least 80%, even more preferably at least 90% or even more preferably at least 95%, or even more preferably 98% or even more preferably at least 99%, or even more preferably 100%. In a most preferred embodiment, the oligonucleotide of the invention consists of a sequence that is complementary to part of dystrophin pre-mRNA as defined herein. As an example, an oligonucleotide may comprise a sequence that is complementary to part of dystrophin pre-mRNA as defined herein and additional flanking sequences. In a more preferred embodiment, the length of said complementary part of said oligonucleotide is of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides. Preferably, additional flanking sequences are used to modify the binding of a protein to the oligonucleotide, or to modify a thermodynamic property of the oligonucleotide, more preferably to modify target RNA binding affinity.

One preferred embodiment provides an oligonucleotide, or a functional equivalent thereof which comprises:
a sequence which is complementary to a region of a dystrophin pre-mRNA exon that is hybridized to another part of a dystrophin pre-mRNA exon (closed structure), and
a sequence which is complementary to a region of a dystrophin pre-mRNA exon that is not hybridized in said dystrophin pre-mRNA (open structure).

For this embodiment, reference is made to WO 2004/083432, which is incorporated by reference in its entirety. RNA molecules exhibit strong secondary structures, mostly due to base pairing of complementary or partly complementary stretches within the same RNA. It has long since been thought that structures in the RNA play a role in the function of the RNA. Without being bound by theory, it is believed that the secondary structure of the RNA of an exon plays a role in structuring the splicing process. The structure of an exon is one parameter which is believed to direct its inclusion into the mRNA. However, other parameters may also play a role therein. Herein this signaling function is referred to as an exon inclusion signal. A complementary oligonucleotide of this embodiment is capable of interfering with the structure of the exon and thereby capable of interfering with the exon inclusion signal of the exon. It has been found that many complementary oligonucleotides indeed comprise this capacity, some more efficient than others. Oligonucleotides of this preferred embodiment, i.e. those with the said overlap directed towards open and closed structures in the native exon RNA, are a selection from all possible oligonucleotides. The selection encompasses oligonucleotides that can efficiently interfere with an exon inclusion signal. Without being bound by theory it is thought that the overlap with an open structure improves the invasion efficiency of the oligonucleotide and prevents the binding of splicing factors (i.e. increases the efficiency with which the oligonucleotide can enter the structure), whereas the overlap with the closed structure subsequently increases the efficiency of interfering with the secondary structure of the RNA of the exon, and thereby interfere with the exon inclusion signal. It is found that the length of the partial complementarity to both the closed and the open structure is not extremely restricted. We have observed high efficiencies with oligonucleotides with variable lengths of complementarity in either structure. The term complementarity is used herein to refer to a stretch of nucleic acids that can hybridise to another stretch of nucleic acids under physiological conditions. It is thus not absolutely required that all the bases in the region of complementarity are capable of pairing with bases in the opposing strand. For instance, when designing the oligonucleotide one may want to incorporate for instance a residue that does not base pair with the base on the complementary strand. Mismatches may, to some extent, be allowed, if under the circumstances in the cell, the stretch of nucleotides is sufficiently capable of hybridising to the complementary part. In this context, “sufficiently” preferably means that using a gel mobility shift assay as described in example 1 of EP 1 619 249, binding of an oligonucleotide is detectable. Optionally, said oligonucleotide may further be tested by transfection into muscle cells of patients. Skipping of the targeted exon may be assessed by RT-PCR (as described in EP 1 619 249). The complementary regions are preferably designed such that, when combined, they are specific for the exon in the pre-mRNA. Such specificity may be created with various lengths of complementary regions as this depends on the actual sequences in other (pre-)mRNA in the system. The risk that also one or more other pre-mRNA will be able to hybridise to the oligonucleotide decreases with increasing size of the oligonucleotide. It is clear that oligonucleotides comprising mismatches in the region of complementarity but that retain the capacity to hybridise to the targeted region(s) in the pre-mRNA, can be used in the present invention. However, preferably at least the complementary parts do not comprise such mismatches as these typically have a higher efficiency and a higher specificity, than oligonucleotides having such mismatches in one or more complementary regions. It is thought, that higher hybridisation strengths, (i.e. increasing number of interactions with the opposing strand) are favourable in increasing the efficiency of the process of interfering with the splicing machinery of the system. Preferably, the complementarity is between 90 and 100%. In general this allows for approximately 1 or 2 mismatch(es) in an oligonucleotide of around 20 nucleotides

The secondary structure is best analysed in the context of the pre-mRNA wherein the exon resides. Such structure may be analysed in the actual RNA. However, it is currently possible to predict the secondary structure of an RNA molecule (at lowest energy costs) quite well using structure-modelling programs. A non-limiting example of a suitable program is RNA mfold version 3.1 server⁴¹. A person skilled in the art will be able to predict, with suitable reproducibility, a likely structure of the exon, given the nucleotide sequence. Best predictions are obtained when providing such modelling programs with both the exon and flanking intron sequences. It is typically not necessary to model the structure of the entire pre-mRNA.

The open and closed structure to which the oligonucleotide is directed, are preferably adjacent to one another. It is thought, that in this way the annealing of the oligonucleotide to the open structure induces opening of the closed structure whereupon annealing progresses into this closed structure. Through this action the previously closed structure assumes a different conformation. The different conformation results in the disruption of the exon inclusion signal. However, when potential (cryptic) splice acceptor and/or donor sequences are present within the targeted exon, occasionally a new exon inclusion signal is generated defining a different (neo) exon, i.e. with a different 5′ end, a different 3′ end, or both. This type of activity is within the scope of the present invention as the targeted exon is excluded from the mRNA. The presence of a new exon, containing part of the targeted exon, in the mRNA does not alter the fact that the targeted exon, as such, is excluded. The inclusion of a neo-exon can be seen as a side effect, which occurs only occasionally. There are two possibilities when exon skipping is used to restore (part of) an open reading frame of dystrophin that is disrupted as a result of a mutation. One is that the neo-exon is functional in the restoration of the reading frame, whereas in the other case the reading frame is not restored. When selecting oligonucleotides for restoring dystrophin reading frames by means of exon-skipping it is of course clear that under these conditions only those oligonucleotides are selected that indeed result in exon-skipping that restores the dystrophin open reading frame, with or without a neo-exon.

Further provided is an oligonucleotide, or a functional equivalent thereof, comprising a sequence that is complementary to a binding site for a serine-arginine (SR) protein in RNA of an exon of a dystrophin pre-mRNA. In WO 2006/112705 we have disclosed the presence of a correlation between the effectivity of an exon-internal antisense oligonucleotide (AON) in inducing exon skipping and the presence of a (for example by ESE finder) predicted SR binding site in the target pre-mRNA site of said AON. Therefore, in one embodiment an oligonucleotide is generated comprising determining a (putative) binding site for an SR (Ser-Arg) protein in RNA of a dystrophin exon and producing an oligonucleotide that is complementary to said RNA and that at least partly overlaps said (putative) binding site. The term “at least partly overlaps” is defined herein as to comprise an overlap of only a single nucleotide of an SR binding site as well as multiple nucleotides of said binding site as well as a complete overlap of said binding site. This embodiment preferably further comprises determining from a secondary structure of said RNA, a region that is hybridised to another part of said RNA (closed structure) and a region that is not hybridised in said structure (open structure), and subsequently generating an oligonucleotide that at least partly overlaps said (putative) binding site and that overlaps at least part of said closed structure and overlaps at least part of said open structure. In this way we increase the chance of obtaining an oligonucleotide that is capable of interfering with the exon inclusion from the pre-mRNA into mRNA. It is possible that a first selected SR-binding region does not have the requested open-closed structure in which case another (second) SR protein binding site is selected which is then subsequently tested for the presence of an open-closed structure. This process is continued until a sequence is identified which contains an SR protein binding site as well as a(n) (partly overlapping) open-closed structure. This sequence is then used to design an oligonucleotide which is complementary to said sequence.

Such a method, for generating an oligonucleotide, is also performed by reversing the described order, i.e. first generating an oligonucleotide comprising determining, from a secondary structure of RNA from a dystrophin exon, a region that assumes a structure that is hybridised to another part of said RNA (closed structure) and a region that is not hybridised in said structure (open structure), and subsequently generating an oligonucleotide, of which at least a part of said oligonucleotide is complementary to said closed structure and of which at least another part of said oligonucleotide is complementary to said open structure. This is then followed by determining whether an SR protein binding site at least overlaps with said open/closed structure. In this way the method of WO 2004/083432 is improved. In yet another embodiment the selections are performed simultaneously.

Without wishing to be bound by any theory it is currently thought that use of an oligonucleotide directed to an SR protein binding site results in (at least partly) impairing the binding of an SR protein to the binding site of an SR protein which results in disrupted or impaired splicing.

Preferably, an open/closed structure and an SR protein binding site partly overlap and even more preferred an open/closed structure completely overlaps an SR protein binding site or an SR protein binding site completely overlaps an open/closed structure. This allows for an improved disruption of exon inclusion.

Besides consensus splice sites sequences, many (if not all) exons contain splicing regulatory sequences such as exonic splicing enhancer (ESE) sequences to facilitate the recognition of genuine splice sites by the spliceosome^{42, 43}. A subgroup of splicing factors, called the SR proteins, can bind to these ESEs and recruit other splicing factors, such as U1 and U2AF to (weakly defined) splice sites. The binding sites of the four most abundant SR proteins (SF2/ASF, SC35, SRp40 and SRp55) have been analyzed in detail and these results are implemented in ESE finder, a web source that predicts potential binding sites for these SR proteins^{42, 43} There is a correlation between the effectiveness of an AON and the presence/absence of an SF2/ASF, SC35 and SRp40 binding site. In a preferred embodiment, the invention thus provides a combination as described above, wherein said SR protein is SF2/ASF or SC35 or SRp40.

In one embodiment an oligonucleotide, or a functional equivalent thereof is capable of specifically binding a regulatory RNA sequence which is required for the correct splicing of a dystrophin exon in a transcript. Several cis-acting RNA sequences are required for the correct splicing of exons in a transcript. In particular, supplementary elements such as intronic or exonic splicing enhancers (ISEs and ESEs) or silencers (ISSs and ESEs) are identified to regulate specific and efficient splicing of constitutive and alternative exons. Using sequence-specific antisense oligonucleotides (AONs) that bind to the elements, their regulatory function is disturbed so that the exon is skipped, as shown for DMD. Hence, in one preferred embodiment an oligonucleotide or functional equivalent thereof is used which is complementary to an intronic splicing enhancer (ISE), an exonic splicing enhancer (ESE), an intronic splicing silencer (ISS) and/or an exonic splicing silencer (ESS). As already described herein before, a dystrophin exon is in one preferred embodiment skipped by an agent capable of specifically inhibiting an exon inclusion signal of said exon, so that said exon is not recognized by the splicing machinery as a part that needs to be included in the mRNA. As a result, a mRNA without said exon is formed.

An AON used herein is preferably complementary to a consecutive part or a contiguous stretch of between 8 and 50 nucleotides of dystrophin exon RNA or dystrophin intron RNA. In one embodiment an AON used herein is complementary to a consecutive part or a contiguous stretch of between 14 and 50 nucleotides of a dystrophin exon RNA or dystrophin intron RNA. Preferably, said AON is complementary to a consecutive part or contiguous stretch of between 14 and 25 nucleotides of said exon RNA. More preferably, an AON is used which comprises a sequence which is complementary to a consecutive part or a contiguous stretch of between 20 and 25 nucleotides of a dystrophin exon RNA or a dystrophin intron RNA.

Different types of nucleic acid may be used to generate an oligonucleotide. Preferably, said oligonucleotide comprises RNA, as RNA/RNA hybrids are very stable. Since one of the aims of the exon skipping technique is to direct splicing in subjects it is preferred that the oligonucleotide RNA comprises a modification providing the RNA with an additional property, for instance resistance to endonucleases, exonucleases, and RNaseH, additional hybridisation strength, increased stability (for instance in a bodily fluid), increased or decreased flexibility, reduced toxicity, increased intracellular transport, tissue-specificity, etc. Preferably, said modification comprises a 2′-O-methyl-phosphorothioate oligoribonucleotide modification. Preferably, said modification comprises a 2′-O-methyl-phosphorothioate oligo deoxyribonucleotide modification. One embodiment thus provides an oligonucleotide is used which comprises RNA which contains a modification, preferably a 2′-O-methyl modified ribose (RNA) or deoxyribose (DNA) modification.

In one embodiment the invention provides a hybrid oligonucleotide comprising an oligonucleotide comprising a 2′-O-methyl-phosphorothioate oligo(deoxy)ribonucleotide modification and locked nucleic acid. This particular oligonucleotide comprises better sequence specificity compared to an equivalent consisting of locked nucleic acid, and comprises improved effectivity when compared with an oligonucleotide consisting of 2′-O-methyl-phosphorothioate oligo(deoxy)ribonucleotide modification.

With the advent of nucleic acid mimicking technology it has become possible to generate molecules that have a similar, preferably the same hybridisation characteristics in kind not necessarily in amount as nucleic acid itself. Such functional equivalents are of course also suitable for use in the invention. Preferred examples of functional equivalents of an oligonucleotide are peptide nucleic acid and/or locked nucleic acid. Most preferably, a morpholino phosphorodiamidate is used. Suitable but non-limiting examples of equivalents of oligonucleotides of the invention can be found in 44-5°. Hybrids between one or more of the equivalents among each other and/or together with nucleic acid are of course also suitable. In a preferred embodiment locked nucleic acid is used as a functional equivalent of an oligonucleotide, as locked nucleic acid displays a higher target affinity and reduced toxicity and therefore shows a higher efficiency of exon skipping.

In one embodiment an oligonucleotide, or a functional equivalent thereof, which is capable of inhibiting inclusion of a dystrophin exon into dystrophin mRNA is combined with at least one other oligonucleotide, or functional equivalent thereof, that is capable of inhibiting inclusion of another dystrophin exon into dystrophin mRNA. This way, inclusion of two or more exons of a dystrophin pre-mRNA in mRNA produced from this pre-mRNA is prevented. This embodiment is further referred to as double- or multi-exon skipping^{2, 15}. In most cases double-exon skipping results in the exclusion of only the two targeted exons from the dystrophin pre-mRNA. However, in other cases it was found that the targeted exons and the entire region in between said exons in said pre-mRNA were not present in the produced mRNA even when other exons (intervening exons) were present in such region. This multi-exon skipping was notably so for the combination of oligonucleotides derived from the DMD gene, wherein one oligonucleotide for exon 45 and one oligonucleotide for exon 51 was added to a cell transcribing the DMD gene. Such a set-up resulted in mRNA being produced that did not contain exons 45 to 51. Apparently, the structure of the pre-mRNA in the presence of the mentioned oligonucleotides was such that the splicing machinery was stimulated to connect exons 44 and 52 to each other. Other preferred examples of multi-exon skipping are:
the use of an oligonucleotide targeting exon 17, and a second one exon 48 which may result in the skipping of said both exons or of the entire region between exon 17 and exon 48.
the use of an oligonucleotide targeting exon 17, and a second one exon 51 which may result in the skipping of said both exons or of the entire region between exon 17 and exon 51.
the use of an oligonucleotide targeting exon 42, and a second one exon 55 which may result in the skipping of said both exons or of the entire region between exon 42 and exon 55.
the use of an oligonucleotide targeting exon 43, and a second one exon 51 which may result in the skipping of said both exons or of the entire region between exon 43 and exon 51.
the use of an oligonucleotide targeting exon 43, and a second one exon 55 which may result in the skipping of said both exons or of the entire region between exon 43 and exon 55.
the use of an oligonucleotide targeting exon 45, and a second one exon 55 which may result in the skipping of said both exons or of the entire region between exon 45 and exon 55.
the use of an oligonucleotide targeting exon 45, and a second one exon 59 which may result in the skipping of said both exons or of the entire region between exon 45 and exon 59.
the use of an oligonucleotide targeting exon 48, and a second one exon 59 which may result in the skipping of said both exons or of the entire region between exon 48 and exon 59.
the use of an oligonucleotide targeting exon 50, and a second one exon 51 which may result in the skipping of said both exons.
the use of an oligonucleotide targeting exon 51, and a second one exon 52 which may result in the skipping of said both exons.

Further provided is therefore an oligonucleotide which comprises at least 8, preferably between 16 to 80, consecutive nucleotides that are complementary to a first exon of a dystrophin pre-mRNA and wherein a nucleotide sequence is used which comprises at least 8, preferably between 16 to 80, consecutive nucleotides that are complementary to a second exon of said dystrophin pre-mRNA. Said first and said second exon may be the same.

In one preferred embodiment said first and said second exon are separated in said dystrophin pre-mRNA by at least one exon to which said oligonucleotide is not complementary. Alternatively, said first and said second exon are adjacent.

It is possible to specifically promote the skipping of also the intervening exons by providing a linkage between the two complementary oligonucleotides. Hence, in one embodiment stretches of nucleotides complementary to at least two dystrophin exons are separated by a linking moiety. The at least two stretches of nucleotides are thus linked in this embodiment so as to form a single molecule. Further provided is therefore an oligonucleotide, or functional equivalent thereof which is complementary to at least two exons in a dystrophin pre-mRNA, said oligonucleotide or functional equivalent comprising at least two parts wherein a first part comprises an oligonucleotide having at least 8, preferably between 16 to 80, consecutive nucleotides that are complementary to a first of said at least two exons and wherein a second part comprises an oligonucleotide having at least 8, preferably between 16 to 80, consecutive nucleotides that are complementary to a second exon in said dystrophin pre-mRNA. The linkage may be through any means, but is preferably accomplished through a nucleotide linkage. In the latter case, the number of nucleotides that do not contain an overlap between one or the other complementary exon can be zero, but is preferably between 4 to 40 nucleotides. The linking moiety can be any type of moiety capable of linking oligonucleotides. Preferably, said linking moiety comprises at least 4 uracil nucleotides. Currently, many different compounds are available that mimic hybridisation characteristics of oligonucleotides. Such a compound, called herein a functional equivalent of an oligonucleotide, is also suitable for the present invention if such equivalent comprises similar hybridisation characteristics in kind not necessarily in amount. Suitable functional equivalents are mentioned earlier in this description. As mentioned, oligonucleotides of the invention do not have to consist of only oligonucleotides that contribute to hybridisation to the targeted exon. There may be additional material and/or nucleotides added.

The DMD gene is a large gene, with many different exons. Considering that the gene is located on the X-chromosome, it is mostly boys that are affected, although girls can also be affected by the disease, as they may receive a bad copy of the gene from both parents, or are suffering from a particularly biased inactivation of the functional allele due to a particularly biased X chromosome inactivation in their muscle cells. The protein is encoded by a plurality of exons (79) over a range of at least 2.4 Mb. Defects may occur in any part of the DMD gene. Skipping of a particular exon or particular exons can, very often, result in a restructured mRNA that encodes a shorter than normal but at least partially functional dystrophin protein. A practical problem in the development of a medicament based on exon-skipping technology is the plurality of mutations that may result in a deficiency in functional dystrophin protein in the cell. Despite the fact that already multiple different mutations can be corrected for by the skipping of a single exon, this plurality of mutations, requires the generation of a series of different pharmaceuticals as for different mutations different exons need to be skipped. An advantage of an oligonucleotide or of a composition comprising at least two distinct oligonucleotide as later defined herein capable of inducing skipping of two or more exons, is that more than one exon can be skipped with a single pharmaceutical. This property is not only practically very useful in that only a limited number of pharmaceuticals need to be generated for treating many different DMD or particular, severe BMD mutations. Another option now open to the person skilled in the art is to select particularly functional restructured dystrophin proteins and produce compounds capable of generating these preferred dystrophin proteins. Such preferred end results are further referred to as mild phenotype dystrophins.

Dose ranges of oligonucleotide according to the invention are preferably designed on the basis of rising dose studies in clinical trials (in vivo use) for which rigorous protocol requirements exist. A molecule or an oligonucleotide as defined herein may be used at a dose which is ranged between 0.1 and 20 mg/kg, preferably 0.5 and 10 mg/kg.

In a preferred embodiment, a concentration of an oligonucleotide as defined herein, which is ranged between 0.1 nM and 1 μM is used. Preferably, this range is for in vitro use in a cellular model such as muscular cells or muscular tissue. More preferably, the concentration used is ranged between 0.3 to 400 nM, even more preferably between 1 to 200 nM. If several oligonucleotides are used, this concentration or dose may refer to the total concentration or dose of oligonucleotides or the concentration or dose of each oligonucleotide added.

The ranges of concentration or dose of oligonucleotide(s) as given above are preferred concentrations or doses for in vitro or ex vivo uses. The skilled person will understand that depending on the oligonucleotide(s) used, the target cell to be treated, the gene target and its expression levels, the medium used and the transfection and incubation conditions, the concentration or dose of oligonucleotide(s) used may further vary and may need to be optimised any further.

An oligonucleotide as defined herein for use according to the invention may be suitable for administration to a cell, tissue and/or an organ in vivo of individuals affected by or at risk of developing DMD or BMD, and may be administered in vivo, ex vivo or in vitro. Said oligonucleotide may be directly or indirectly administrated to a cell, tissue and/or an organ in vivo of an individual affected by or at risk of developing DMD or BMD, and may be administered directly or indirectly in vivo, ex vivo or in vitro. As Duchenne and Becker muscular dystrophy have a pronounced phenotype in muscle cells, it is preferred that said cells are muscle cells, it is further preferred that said tissue is a muscular tissue and/or it is further preferred that said organ comprises or consists of a muscular tissue. A preferred organ is the heart. Preferably, said cells comprise a gene encoding a mutant dystrophin protein. Preferably, said cells are cells of an individual suffering from DMD or BMD.

An oligonucleotide of the invention may be indirectly administrated using suitable means known in the art. An oligonucleotide may for example be provided to an individual or a cell, tissue or organ of said individual in the form of an expression vector wherein the expression vector encodes a transcript comprising said oligonucleotide. The expression vector is preferably introduced into a cell, tissue, organ or individual via a gene delivery vehicle. In a preferred embodiment, there is provided a viral-based expression vector comprising an expression cassette or a transcription cassette that drives expression or transcription of a molecule as identified herein. A preferred delivery vehicle is a viral vector such as an adeno-associated virus vector (AAV), or a retroviral vector such as a lentivirus vector^{4, 51, 52} and the like. Also, plasmids, artificial chromosomes, plasmids suitable for targeted homologous recombination and integration in the human genome of cells may be suitably applied for delivery of an oligonucleotide as defined herein. Preferred for the current invention are those vectors wherein transcription is driven from PolIII promoters, and/or wherein transcripts are in the form fusions with U1 or U7 transcripts, which yield good results for delivering small transcripts. It is within the skill of the artisan to design suitable transcripts. Preferred are PolIII driven transcripts. Preferably, in the form of a fusion transcript with an U1 or U7 transcript^{4, 51, 52} Such fusions may be generated as described^{53, 54}. The oligonucleotide may be delivered as is. However, the oligonucleotide may also be encoded by the viral vector. Typically, this is in the form of an RNA transcript that comprises the sequence of the oligonucleotide in a part of the transcript.

Improvements in means for providing an individual or a cell, tissue, organ of said individual with an oligonucleotide and/or an equivalent thereof, are anticipated considering the progress that has already thus far been achieved. Such future improvements may of course be incorporated to achieve the mentioned effect on restructuring of mRNA using a method of the invention. An oligonucleotide and/or an equivalent thereof can be delivered as is to an individual, a cell, tissue or organ of said individual. When administering an oligonucleotide and/or an equivalent thereof, it is preferred that an oligonucleotide and/or an equivalent thereof is dissolved in a solution that is compatible with the delivery method. For intravenous, subcutaneous, intramuscular, intrathecal and/or intraventricular administration it is preferred that the solution is a physiological salt solution. Particularly preferred in the invention is the use of an excipient that will aid in delivery of each of the constituents as defined herein to a cell and/or into a cell, preferably a muscle cell. Preferred are excipients capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients comprise polyethylenimine (PEI), or similar cationic polymers, including polypropyleneimine or polyethylenimine copolymers (PECs) and derivatives, synthetic amphiphils (SAINT-18), Lipofectin™, DOTAP and/or viral capsid proteins that are capable of self assembly into particles that can deliver each constitutent as defined herein to a cell, preferably a muscle cell. Such excipients have been shown to efficiently deliver an oligonucleotide such as antisense nucleic acids to a wide variety of cultured cells, including muscle cells. Their high transfection potential is combined with an excepted low to moderate toxicity in terms of overall cell survival. The ease of structural modification can be used to allow further modifications and the analysis of their further (in vivo) nucleic acid transfer characteristics and toxicity.

Lipofectin represents an example of a liposomal transfection agent. It consists of two lipid components, a cationic lipid N-[1-(2,3 dioleoyloxy)propyl]-N,N,N-trimethylammonium chloride (DOTMA) (cp. DOTAP which is the methylsulfate salt) and a neutral lipid dioleoylphosphatidylethanolamine (DOPE). The neutral component mediates the intracellular release. Another group of delivery systems are polymeric nanoparticles.

Polycations such like diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver each constituent as defined herein, preferably an oligonucleotide across cell membranes into cells.

In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an oligonucleotide. The skilled person may select and adapt any of the above or other commercially available alternative excipients and delivery systems to package and deliver an oligonucleotide for use in the current invention to deliver it for the treatment of Duchenne Muscular Dystrophy or Becker Muscular Dystrophy in humans.

In addition, an oligonucleotide could be covalently or non-covalently linked to a targeting ligand specifically designed to facilitate the uptake in to the cell, cytoplasm and/or its nucleus. Such ligand could comprise (i) a compound (including but not limited to peptide(-like) structures) recognising cell, tissue or organ specific elements facilitating cellular uptake and/or (ii) a chemical compound able to facilitate the uptake in to cells and/or the intracellular release of an oligonucleotide from vesicles, e.g. endosomes or lysosomes.

Therefore, in a preferred embodiment, an oligonucleotide is formulated in a composition or a medicament or a composition, which is provided with at least an excipient and/or a targeting ligand for delivery and/or a delivery device thereof to a cell and/or enhancing its intracellular delivery. Accordingly, the invention also encompasses a pharmaceutically acceptable composition comprising an oligonucleotide and further comprising at least one excipient and/or a targeting ligand for delivery and/or a delivery device of said oligonucleotide to a cell and/or enhancing its intracellular delivery. It is to be understood that if a composition comprises an additional constituent such as an adjunct compound as later defined herein, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an oligonucleotide and a further adjunct compound as later defined herein.

A preferred oligonucleotide is for preventing or treating Duchenne Muscular Dystrophy (DMD) or Becker Muscular Dystrophy (BMD) in an individual. An individual, which may be treated using an oligonucleotide of the invention may already have been diagnosed as having a DMD or a BMD. Alternatively, an individual which may be treated using an oligonucleotide of the invention may not have yet been diagnosed as having a DMD or a BMD but may be an individual having an increased risk of developing a DMD or a BMD in the future given his or her genetic background. A preferred individual is a human being.

### Composition

In a further aspect, there is provided a composition comprising an oligonucleotide as defined herein. Preferably, said composition comprises at least two distinct oligonucleotide as defined herein. More preferably, these two distinct oligonucleotides are designed to skip distinct two or more exons as earlier defined herein for multi-exon skipping.

In a preferred embodiment, said composition being preferably a pharmaceutical composition said pharmaceutical composition comprising a pharmaceutically acceptable carrier, adjuvant, diluent and/or excipient. Such a pharmaceutical composition may comprise any pharmaceutically acceptable carrier, filler, preservative, adjuvant, solubilizer, diluent and/or excipient is also provided. Such pharmaceutically acceptable carrier, filler, preservative, adjuvant, solubilizer, diluent and/or excipient may for instance be found in Remington: The Science and Practice of Pharmacy, 20th Edition. Baltimore, Md.: Lippincott Williams & Wilkins, 2000. Each feature of said composition has earlier been defined herein.

If several oligonucleotides are used, concentration or dose already defined herein may refer to the total concentration or dose of all oligonucleotides used or the concentration or dose of each oligonucleotide used or added. Therefore in one embodiment, there is provided a composition wherein each or the total amount of oligonucleotide used is dosed in an amount ranged between 0.5 mg/kg and 10 mg/kg.

A preferred composition additionally comprises:
a) an adjunct compound for reducing inflammation, preferably for reducing muscle tissue inflammation, and/or
b) an adjunct compound for improving muscle fiber function, integrity and/or survival and/or
c) a compound exhibiting readthrough activity.

It has surprisingly been found that the skipping frequency of a dystrophin exon from a pre-MRNA comprising said exon, when using an oligonucleotide directed toward the exon or to one or both splice sites of said exon, is enhanced if cells expressing said pre-mRNA are also provided with an adjunct compound for reducing inflammation, preferably for reducing muscle tissue inflammation, and/or an adjunct compound for improving muscle fiber function, integrity and/or survival. The enhanced skipping frequency also increases the level of functional dystrophin protein produced in a muscle cell of a DMD or BMD individual.

According to the present invention, even when a dystrophin protein deficiency has been restored in a DMD patient by administering an oligonucleotide of the invention, the presence of tissue inflammation and damaged muscle cells still continues to contribute to the symptoms of DMD. Hence, even though the cause of DMD—i.e. a dysfunctional dystrophin protein—is alleviated, treatment of DMD is still further improved by additionally using an adjunct therapy according to the present invention. Furthermore, the present invention provides the insight that a reduction of inflammation does not result in significant reduction of AON uptake by muscle cells. This is surprising because, in general, inflammation enhances the trafficking of cells, blood and other compounds. As a result, AON uptake/delivery is also enhanced during inflammation. Hence, before the present invention it would be expected that an adjunct therapy counteracting inflammation involves the risk of negatively influencing AON therapy. This, however, appears not to be the case.

An adjunct compound for reducing inflammation comprises any therapy which is capable of at least in part reducing inflammation, preferably inflammation caused by damaged muscle cells. Said adjunct compound is most preferably capable of reducing muscle tissue inflammation. Inflammation is preferably assessed by detecting an increase in the number of infiltrating immune cells such as neutrophils and/or mast cells and/or dendritic cells and/or lymphocytes in muscle tissue suspected to be dystrophic. This assessment is preferably carried out in cross-sections of a biopsy⁵⁷ of muscle tissue suspected to be dystrophic after having specifically stained immune cells as identified above. The quantification is preferably carried out under the microscope. Reducing inflammation is therefore preferably assessed by detecting a decrease in the number of immune cells in a cross-section of muscle tissue suspected to be dystrophic. Detecting a decrease preferably means that the number of at least one sort of immune cells as identified above is decreased of at least 1%, 2%, 3%, 5%, 7%, 10%, 12%, 15%, 17%, 20%, 30%, 40%, 50%, 60%, 70%, 80%, 90% or more compared to the number of a corresponding immune cell in a same individual before treatment. Most preferably, no infiltrating immune cells are detected in cross-sections of said biopsy.

An adjunct compound for improving muscle fiber function, integrity and/or survival comprises any therapy, which is capable of measurably enhancing muscle fiber function, integrity and/or survival as compared to an otherwise similar situation wherein said adjunct compound is not present. The improvement of muscle fiber function, integrity and/or survival may be assessed using at least one of the following assays: a detectable decrease of creatine kinase in blood, a detectable decrease of necrosis of muscle fibers in a biopsy cross-section of a muscle suspected to be dystrophic, and/or a detectable increase of the homogeneity of the diameter of muscle fibers in a biopsy cross-section of a muscle suspected to be dystrophic. Each of these assays is known to the skilled person.

Creatine kinase may be detected in blood as described in 57. A detectable decrease in creatine kinase may mean a decrease of 5%, 10%, 20%, 30%, 40%, 50%, 60%, 70%, 80%, 90% or more compared to the concentration of creatine kinase in a same individual before treatment.

A detectable decrease of necrosis of muscle fibers is preferably assessed in a muscle biopsy, more preferably as described in 57 using biopsy cross-sections. A detectable decrease of necrosis may be a decrease of 5%, 10%, 20%, 30%, 40%, 50%, 60%, 70%, 80%, 90% or more of the area wherein necrosis has been identified using biopsy cross-sections. The decrease is measured by comparison to the necrosis as assessed in a same individual before treatment.

A detectable increase of the homogeneity of the diameter of a muscle fiber is preferably assessed in a muscle biopsy cross-section, more preferably as described in 57.

In one embodiment, an adjunct compound for increasing turnover of damaged muscle cells is used. An adjunct compound for increasing turnover of damaged muscle cells comprises any therapy, which is capable of at least in part inducing and/or increasing turnover of damaged muscle cells. Damaged muscle cells are muscle cells, which have significantly less clinically measurable functionality than a healthy, intact muscle cell. In the absence of dystrophin, mechanical stress leads to sarcolemmal ruptures, causing an uncontrolled influx of calcium into the muscle fiber interior, thereby triggering calcium-activated proteases and fiber necrosis, resulting in damaged muscle cells. Increasing turnover of damaged muscle cells means that damaged muscle cells are more quickly broken down and/or removed as compared to a situation wherein turnover of damaged muscle cells is not increased. Turnover of damaged muscle cells is preferably assessed in a muscle biopsy, more preferably as described in 57 using a cross-section of a biopsy. A detectable increase of turnover may be an increase of 5%, 10%, 20%, 30%, 40%, 50%, 60%, 70%, 80%, 90% or more of the area wherein turnover has been identified using a biopsy cross-section. The increase is measured by comparison to the turnover as assessed in a same individual before treatment.

Without wishing to be bound to theory, it is believed that increasing turnover of muscle cells is preferred because this reduces inflammatory responses.

According to the present invention, a composition of the invention further comprising an adjunct therapy for reducing inflammation, preferably for reducing muscle tissue inflammation in an individual, is particularly suitable for use as a medicament. Such composition is even better capable of alleviating one or more symptom(s) of Duchenne Muscular Dystrophy or Becker Muscular Dystrophy as compared to a combination not comprising said adjunct compound. This embodiment also enhances the skipping frequency of a dystrophin exon from a pre-mRNA comprising said exon, when using an oligonucleotide directed toward the exon or to one or both splice sites of said exon. The enhanced skipping frequency also increases the level of functional dystrophin protein produced in a muscle cell of a DMD or BMD individual.

Further provided is therefore a composition further comprising an adjunct compound for reducing inflammation, preferably for reducing muscle tissue inflammation in said individual, for use as a medicament, preferably for treating or preventing counteracting DMD. In one embodiment, said composition is used in order to alleviate one or more symptom(s) of a severe form of BMD wherein a very short dystrophin protein or altered or truncated dystrophin mRNA or protein is formed which is not sufficiently functional.

Preferred adjunct compound for reducing inflammation include a steroid, a TNFα inhibitor, a source of mIGF-1 and/or an antioxidant. However, any other compound able to reduce inflammation as defined herein is also encompassed within the present invention. Each of these compounds is later on extensively presented. Each of the compounds extensively presented may be used separately or in combination with each other and/or in combination with one or more of the adjunct compounds used for improving muscle fiber function, integrity and/or survival.

Furthermore, a composition comprising an adjunct therapy for improving muscle fiber function, integrity and/or survival in an individual is particularly suitable for use as a medicament, preferably for treating or preventing DMD. Such composition is even better capable of alleviating one or more symptom(s) of Duchenne Muscular Dystrophy as compared to a composition not comprising said adjunct compound.

Preferred adjunct compounds for improving muscle fiber function, integrity and/or survival include an ion channel inhibitor, a protease inhibitor, L-arginine and/or an angiotensin II type I receptor blocker. However, any other compound able to improving muscle fiber function, integrity and/or survival as defined herein is also encompassed within the present invention. Each of these compounds is later on extensively presented. Each of the compounds extensively presented may be used separately or in combination with each other and/or in combination with one or more of the adjunct compounds used for reducing inflammation.

In a particularly preferred embodiment, a composition further comprises a steroid. Such composition results in significant alleviation of DMD symptoms. This embodiment also enhances the skipping frequency of a dystrophin exon from a pre-mRNA comprising said exon, when using an oligonucleotide directed toward the exon or to one or both splice sites of said exon. The enhanced skipping frequency also increases the level of functional dystrophin protein produced in a muscle cell of a DMD or BMD individual.

In one embodiment, said composition is used in order to alleviate one or more symptom(s) of a severe form of BMD wherein a very short dystrophin protein is formed which is not sufficiently functional.

A steroid is a terpenoid lipid characterized by a carbon skeleton with four fused rings, generally arranged in a 6-6-6-5 fashion. Steroids vary by the functional groups attached to these rings and the oxidation state of the rings. Steroids include hormones and drugs, which are usually used to relieve swelling and inflammation, such as for instance prednisone, dexamethasone and vitamin D.

According to the present invention, supplemental effects of adjunct steroid therapy in DMD patients include reduction of tissue inflammation, suppression of cytotoxic cells, and improved calcium homeostasis. Most positive results are obtained in younger boys. Preferably, the steroid is a corticosteroid, more preferably, a glucocorticosteroid. Preferably, prednisone steroids such as prednisone, prednizolone or deflazacort are used in a combination according to the invention²¹. Dose ranges of steroid or of a glucocorticosteroid to be used in the therapeutic applications as described herein are designed on the basis of rising dose studies in clinical trials for which rigorous protocol requirements exist. The usual doses are 0.5-1.0 mg/kg/day, preferably 0.75 mg/kg/day for prednisone and prednisolone, and 0.4-1.4 mg/kg/day, preferably 0.9 mg/kg/day for deflazacort.

In one embodiment, a steroid is administered to said individual prior to administering a composition as earlier defined herein. In this embodiment, it is preferred that said steroid is administered at least one day, more preferred at least one week, more preferred at least two weeks, more preferred at least three weeks prior to administering said composition.

In another preferred embodiment, a combination further comprises a tumour necrosis factor-alpha (TNFα) inhibitor. Tumour necrosis factor-alpha (TNFα) is a pro-inflammatory cytokine that stimulates the inflammatory response. Pharmacological blockade of TNFα activity with the neutralising antibody infliximab (Remicade) is highly effective clinically at reducing symptoms of inflammatory diseases. In mdx mice, both infliximab and etanercept delay and reduce the necrosis of dystrophic muscle^{24, 25}, with additional physiological benefits on muscle strength, chloride channel function and reduced CK levels being demonstrated in chronically treated exercised adult mdx mice²⁶. Such highly specific anti-inflammatory drugs designed for use in other clinical conditions, are attractive alternatives to the use of steroids for DMD. In one embodiment, the use of a TNFα inhibitor is limited to periods of intensive muscle growth in boys when muscle damage and deterioration are especially pronounced.

A composition further comprising a TNFα inhibitor for use as a medicament is also provided. In one embodiment, said composition is used in order to alleviate one or more symptom(s) of a severe form of BMD wherein a very short dystrophin protein is formed which is not sufficiently functional. A preferred TNFα inhibitor is a dimeric fusion protein consisting of the extracellular ligand-binding domain of the human p75 receptor of TNFα linked to the Fc portion of human IgG1. A more preferred TNFα inhibitor is ethanercept (Amgen, America)²⁶. The usual doses of ethanercept is about 0.2 mg/kg, preferably about 0.5 mg/kg twice a week. The administration is preferably subcutaneous.

In another preferred embodiment, a composition of the invention further comprises a source of mIGF-1. As defined herein, a source of IGF-1 preferably encompasses mIGF-1 itself, a compound able of enhancing mIGF-1 expression and/or activity. Enhancing is herein synonymous with increasing. Expression of mIGF-1 is synonymous with amount of mIGF-1. mIGF-1 promotes regeneration of muscles through increase in satellite cell activity, and reduces inflammation and fibrosis²⁷. Local injury of muscle results in increased mIGF-1 expression. In transgenic mice with extra IGF-1 genes, muscle hypertrophy and enlarged muscle fibers are observed²⁷. Similarly, transgenic mdx mice show reduced muscle fiber degeneration²⁸. Upregulation of the mIGF-1 gene and/or administration of extra amounts of mIGF-1 protein or a functional equivalent thereof (especially the mIGF-1 Ea isoform (as described in 27, human homolog IGF-1 isoform 4: SEQ ID NO: 577)) thus promotes the effect of other, preferably genetic, therapies for DMD, including antisense-induced exon skipping. The additional mIGF-1 levels in the above mentioned transgenic mice do not induce cardiac problems nor promote cancer, and have no pathological side effects. As stated before, the amount of mIGF-1 is for instance increased by enhancing expression of the mIGF-1 gene and/or by administration of mIGF-1 protein and/or a functional equivalent thereof (especially the mIGF-1 Ea isoform (as described in 27, human homolog IGF-1 isoform 4: SEQ ID NO: 577)). A composition of the invention further preferably comprises mIGF-1, a compound capable of enhancing mIGF-1 expression and/or an mIGF-1 activity, for use as a medicament is also provided. Said medicament is preferably for alleviating one or more symptom(s) of DMD. In one embodiment, such composition is used in order to alleviate one or more symptom(s) of a severe form of BMD wherein a very short dystrophin protein is formed which is not sufficiently functional.

Within the context of the invention, an increased amount or activity of mIGF-1 may be reached by increasing the gene expression level of an IGF-1 gene, by increasing the amount of a corresponding IGF-1 protein and/or by increasing an activity of an IGF1-protein. A preferred mIGF-1 protein has been earlier defined herein. An increase of an activity of said protein is herein understood to mean any detectable change in a biological activity exerted by said protein or in the steady state level of said protein as compared to said activity or steady-state in a individual who has not been treated. Increased amount or activity of mIGF-1 is preferably assessed by detection of increased expression of muscle hypertrophy biomarker GATA-2 (as described in 27).

Gene expression level is preferably assessed using classical molecular biology techniques such as (real time) PCR, arrays or Northern analysis. A steady state level of a protein is determined directly by quantifying the amount of a protein. Quantifying a protein amount may be carried out by any known technique such as Western blotting or immunoassay using an antibody raised against a protein. The skilled person will understand that alternatively or in combination with the quantification of a gene expression level and/or a corresponding protein, the quantification of a substrate of a corresponding protein or of any compound known to be associated with a function or activity of a corresponding protein or the quantification of said function or activity of a corresponding protein using a specific assay may be used to assess the alteration of an activity or steady state level of a protein.

In the invention, an activity or steady-state level of a said protein may be altered at the level of the protein itself, e.g. by providing a protein to a cell from an exogenous source.

Preferably, an increase or an up-regulation of the expression level of a said gene means an increase of at least 5% of the expression level of said gene using arrays. More preferably, an increase of the expression level of said gene means an increase of at least 10%, even more preferably at least 20%, at least 30%, at least 40%, at least 50%, at least 70%, at least 90%, at least 150% or more. In another preferred embodiment, an increase of the expression level of said protein means an increase of at least 5% of the expression level of said protein using Western blotting and/or using ELISA or a suitable assay. More preferably, an increase of the expression level of a protein means an increase of at least 10%, even more preferably at least 20%, at least 30%, at least 40%, at least 50%, at least 70%, at least 90%, at least 150% or more.

In another preferred embodiment, an increase of a polypeptide activity means an increase of at least 5% of a polypeptide activity using a suitable assay. More preferably, an increase of a polypeptide activity means an increase of at least 10%, even more preferably at least 20%, at least 30%, at least 40%, at least 50%, at least 70%, at least 90%, at least 150% or more. The increase is preferably assessed by comparison to corresponding activity in the individual before treatment.

A preferred way of providing a source of mIGF1 is to introduce a transgene encoding mIGF1, preferably an mIGF-1 Ea isoform (as described in 27, human homolog IGF-1 isoform 4: SEQ ID NO: 577), more preferably in an AAV vector as later defined herein. Such source of mIGF1 is specifically expressed in muscle tissue as described in mice in 27.

In another preferred embodiment, a composition further comprises an antioxidant. Oxidative stress is an important factor in the progression of DMD and promotes chronic inflammation and fibrosis²⁹. The most prevalent products of oxidative stress, the peroxidized lipids, are increased by an average of 35% in Duchenne boys. Increased levels of the enzymes superoxide dismutase and catalase reduce the excessive amount of free radicals causing these effects. In fact, a dietary supplement Protandim® (LifeVantage) was clinically tested and found to increase levels of superoxide dismutase (up to 30%) and catalase (up to 54%), which indeed significantly inhibited the peroxidation of lipids in 29 healthy persons³⁰. Such effective management of oxidative stress thus preserves muscle quality and so promotes the positive effect of DMD therapy. Idebenone is another potent antioxidant with a chemical structure derived from natural coenzyme Q10. It protects mitochondria where adenosine triphosphate, ATP, is generated by oxidative phosphorylation. The absence of dystrophin in DMD negatively affects this process in the heart, and probably also in skeletal muscle. Idebenone was recently applied in clinical trials in the US and Europe demonstrating efficacy on neurological aspects of Friedreich's Ataxia³¹. A phase-IIa double-blind, placebo-controlled randomized clinical trial with Idebenone has recently been started in Belgium, including 21 Duchenne boys at 8 to 16 years of age. The primary objective of this study is to determine the effect of Idebenone on heart muscle function. In addition, several different tests will be performed to detect the possible functional benefit on muscle strength in the patients. When effective, Idebenone is a preferred adjunct compound for use in a combination according to the present invention in order to enhance the therapeutic effect of DMD therapy, especially in the heart. A composition further comprising an antioxidant for use as a medicament is also provided. Said medicament is preferably for alleviating one or more symptom(s) of DMD. In one embodiment, said composition is used in order to alleviate one or more symptom(s) of a severe form of BMD wherein a very short dystrophin protein is formed which is not sufficiently functional. Depending on the identity of the antioxidant, the skilled person will know which quantities are preferably used. An antioxidant may include bacoside, silymarin, curcumin and/or a polyphenol. Preferably, a polyphenol is or comprises epigallocatechin-3-gallate (EGCG). Preferably, an antioxidant is a mixture of antioxidants as the dietary supplement Protandim® (LifeVantage). A daily capsule of 675 mg of Protandim® comprises 150 mg of B. monniera (45% bacosides), 225 mg of S. marianum (70-80% silymarin), 150 mg of W. somnifera powder, 75 mg green tea (98% polyphenols wherein 45% EGCG) and 75 mg turmeric (95% curcumin).

In another preferred embodiment, a composition further comprises an ion channel inhibitor. The presence of damaged muscle membranes in DMD disturbs the passage of calcium ions into the myofibers, and the consequently disrupted calcium homeostasis activates many enzymes, e.g. proteases, that cause additional damage and muscle necrosis. Ion channels that directly contribute to the pathological accumulation of calcium in dystrophic muscle are potential targets for adjunct compounds to treat DMD. There is evidence that some drugs, such as pentoxifylline, block exercise-sensitive calcium channels³² and antibiotics that block stretch activated channels reduce myofibre necrosis in mdx mice and CK levels in DMD boys³³. A composition further comprising an ion channel inhibitor for use as a medicament is also provided. Said medicament is preferably for alleviating one or more symptom(s) of DMD. In one embodiment, said composition is used in order to alleviate one or more symptom(s) of a severe form of BMD wherein a very short dystrophin protein is formed which is not sufficiently functional.

Preferably, an ion channel inhibitor of the class of xanthines is used. More preferably, said xanthines are derivatives of methylxanthines, and most preferably, said methylxanthine derivates are chosen from the group consisting of pentoxifylline, furafylline, lisofylline, propentofylline, pentifylline, theophylline, torbafylline, albifylline, enprofylline and derivatives thereof. Most preferred is the use of pentoxifylline. Ion channel inhibitors of the class of xanthines enhance the skipping frequency of a dystrophin exon from a pre-mRNA comprising said exon, when using an oligonucleotide directed toward the exon or to one or both splice sites of said exon. The enhanced skipping frequency also increases the level of functional dystrophin protein produced in a muscle cell of a DMD or BMD individual.

Depending on the identity of the ion channel inhibitor, the skilled person will know which quantities are preferably used. Suitable dosages of pentoxifylline are between 1 mg/kg/day to 100 mg/kg/day, preferred dosages are between 10 mg/kg/day to 50 mg/kg/day. Typical dosages used in humans are 20 mg/kg/day.

In one embodiment, an ion channel inhibitor is administered to said individual prior to administering a composition comprising an oligonucleotide. In this embodiment, it is preferred that said ion channel inhibitor is administered at least one day, more preferred at least one week, more preferred at least two weeks, more preferred at least three weeks prior to administering a composition comprising an oligonucleotide.

In another preferred embodiment, a composition further comprises a protease inhibitor. Calpains are calcium-activated proteases that are increased in dystrophic muscle and account for myofiber degeneration. Calpain inhibitors such as calpastatin, leupeptin³⁴, calpeptin, calpain inhibitor III, or PD150606 are therefore applied to reduce the degeneration process. A new compound, BN 82270 (Ipsen) that has dual action as both a calpain inhibitor and an antioxidant increased muscle strength, decreased serum CK and reduced fibrosis of the mdx diaphragm, indicating a therapeutic effect with this new compound³⁵. Another compound of Leupeptin/Carnitine (Myodur) has recently been proposed for clinical trials in DMD patients.

MG132 is another proteasomal inhibitor that has shown to reduce muscle membrane damage, and to ameliorate the histopathological signs of muscular dystrophy³⁶. MG-132 (CBZ-leucyl-leucyl-leucinal) is a cell-permeable, proteasomal inhibitor (Ki=4 nM), which inhibits NFkappaB activation by preventing IkappaB degradation (1050=3 μM). In addition, it is a peptide aldehyde that inhibits ubiquitin-mediated proteolysis by binding to and inactivating 20S and 26S proteasomes. MG-132 has shown to inhibit the proteasomal degradation of dystrophin-associated proteins in the dystrophic mdx mouse model³⁶. This compound is thus also suitable for use as an adjunct pharmacological compound for DMD. A composition further comprising a protease inhibitor for use as a medicament is also provided. Said medicament is preferably for alleviating one or more symptom(s) of DMD. In one embodiment, said combination is used in order to alleviate one or more symptom(s) of a severe form of BMD wherein a very short dystrophin protein is formed which is not sufficiently functional. Depending on the identity of the protease inhibitor, the skilled person will know which quantities are preferably used.

In another preferred embodiment, a composition further comprises L-arginine. Dystrophin-deficiency is associated with the loss of the DGC-complex at the fiber membranes, including neuronal nitric oxide synthase (nNOS). Expression of a nNOS transgene in mdx mice greatly reduced muscle membrane damage. Similarly, administration of L-arginine (the substrate for nitric oxide synthase) increased NO production and upregulated utrophin expression in mdx mice. Six weeks of L-arginine treatment improved muscle pathology and decreased serum CK in mdx mice³⁷. The use of L-arginine as a further constituent in a composition of the invention has not been disclosed.

A composition further comprising L-arginine for use as a medicament is also provided. Said medicament is preferably for alleviating one or more symptom(s) of DMD. In one embodiment, said composition is used in order to alleviate one or more symptom(s) of a severe form of BMD wherein a very short dystrophin protein is formed which is not sufficiently functional.

In another preferred embodiment, a composition further comprises angiotensin II type 1 receptor blocker Losartan, which normalizes muscle architecture, repair and function, as shown in the dystrophin-deficient mdx mouse model²³. A composition further comprising angiotensin II type 1 receptor blocker Losartan for use as a medicament is also provided. Said medicament is preferably for alleviating one or more symptom(s) of DMD. In one embodiment, said composition is used in order to alleviate one or more symptom(s) of a severe form of BMD wherein a very short dystrophin protein is formed which is not sufficiently functional. Depending on the identity of the angiotensin II type 1 receptor blocker, the skilled person will know which quantities are preferably used.

In another preferred embodiment, a composition further comprises an angiotensin-converting enzyme (ACE) inhibitor, preferably perindopril. ACE inhibitors are capable of lowering blood pressure. Early initiation of treatment with perindopril is associated with a lower mortality in DMD patients²². A composition further comprising an ACE inhibitor, preferably perindopril for use as a medicament is also provided. Said medicament is preferably for alleviating one or more symptom(s) of DMD. In one embodiment, said composition is used in order to alleviate one or more symptom(s) of a severe form of BMD wherein a very short dystrophin protein is formed which is not sufficiently functional. The usual doses of an ACE inhibitor, preferably perindopril are about 2 to 4 mg/day²². In a more preferred embodiment, an ACE inhibitor is combined with at least one of the previously identified adjunct compounds.

In another preferred embodiment, a composition further comprises a compound exhibiting a readthrough activity. A compound exhibiting a readthrough activity may be any compound, which is able to suppress a stop codon. For 20% of DMD patients, the mutation in the dystrophin gene is comprising a point mutation, of which 13% is a nonsense mutation. A compound exhibiting a readthrough activity or which is able to suppress a stop codon is a compound which is able to provide an increased amount of a functional dystrophin mRNA or protein and/or a decreased amount of an aberrant or truncated dystrophin mRNA or protein. Increased preferably means increased of at least 1%, 2%, 5%, 10%, 15%, 20%, 25%, 30%, 35%, 40%, 45%, 50%, 55%, 60%, 65%, 70%, 75%, 80%, 85%, 90%, 95%, 100% or more. Decreased preferably means decreased of at least 1%, 2%, 5%, 10%, 15%, 20%, 25%, 30%, 35%, 40%, 45%, 50%, 55%, 60%, 65%, 70%, 75%, 80%, 85%, 90%, 95%, 100% or more. An increase or a decrease of said protein is preferably assessed in a muscular tissue or in a muscular cell of an individual by comparison to the amount present in said individual before treatment with said compound exhibiting a readthrough activity. Alternatively, the comparison can be made with a muscular tissue or cell of said individual, which has not yet been treated with said compound in case the treatment is local. The assessment of an amount at the protein level is preferably carried out using western blot analysis.

Preferred compounds exhibiting a readthrough activity comprise or consist of aminoglycosides, including, but not limited to, geneticin (G418), paromomycin, gentamycin and/or 3-(5-(2-fluorophenyl)-1,2,4-oxadiazol-3-yl)benzoic acid), and derivatives thereof (references 64, 65). A more preferred compound exhibiting a readthrough activity comprises or consists of PTC124™, and/or a functional equivalent thereof. PTC124™ is a registered trademark of PTC Therapeutics, Inc. South Plainfield, N.J. 3-(5-(2-fluorophenyl)-1,2,4-oxadiazol-3-yl)benzoic acid) also known as PTC124™ (references 16, 17) belongs to a new class of small molecules that mimics at lower concentrations the readthrough activity of gentamicin (reference 55). A functional equivalent of 3-(5-(2-fluorophenyl)-1,2,4-oxadiazol-3-yl)benzoic acid) or of gentamicin is a compound which is able to exhibit a readthrough activity as earlier defined herein. Most preferably, a compound exhibiting a readthrough activity comprises or consists of gentamycin and/or 3-(5-(2-fluorophenyl)-1,2,4-oxadiazol-3-yl)benzoic acid) also known as PTC124™. A composition further comprising a compound exhibiting a readthrough activity, preferably comprising or consisting of gentamycin and/or 3-(5-(2-fluorophenyl)-1,2,4-oxadiazol-3-yl)benzoic acid) for use as a medicament is also provided. Said medicament is preferably for alleviating one or more symptom(s) of DMD. In one embodiment, said composition is used in order to alleviate one or more symptom(s) of a severe form of BMD wherein a very short dystrophin protein is formed which is not sufficiently functional. The usual doses of a compound exhibiting a readthrough activity, preferably 3-(5-(2-fluorophenyl)-1,2,4-oxadiazol-3-yl)benzoic acid) or of gentamicin are ranged between 3 mg/kg/day to 200 mg/kg/day, preferred dosages are between 10 mg/kg to 50 mg/kg per day or twice a day.

In a more preferred embodiment, a compound exhibiting a readthrough activity is combined with at least one of the previously identified adjunct compounds.

In another preferred embodiment, a composition further comprises a compound, which is capable of enhancing exon skipping and/or inhibiting spliceosome assembly and/or splicing. Small chemical compounds, such as for instance specific indole derivatives, have been shown to selectively inhibit spliceosome assembly and splicing″, for instance by interfering with the binding of serine- and arginine-rich (SR) proteins to their cognate splicing enhancers (ISEs or ESEs) and/or by interfering with the binding of splicing repressors to silencer sequences (ESSs or ISSs). These compounds are therefore suitable for applying as adjunct compounds that enhance exon skipping. A composition further comprising a compound for enhancing exon skipping and/or inhibiting spliceosome assembly and/or splicing for use as a medicament is also provided. Said medicament is preferably for alleviating one or more symptom(s) of DMD. In one embodiment, said composition is used in order to alleviate one or more symptom(s) of a severe form of BMD wherein a very short dystrophin protein is formed which is not sufficiently functional. Depending on the identity of the compound, which is capable of enhancing exon skipping and/or inhibiting spliceosome assembly and/or splicing, the skilled person will know which quantities are preferably used. In a more preferred embodiment, a compound for enhancing exon skipping and/or inhibiting spliceosome assembly and/or splicing is combined with a ACE inhibitor and/or with any adjunct compounds as identified earlier herein.

The invention thus provides a composition further comprising an adjunct compound, wherein said adjunct compound comprises a steroid, an ACE inhibitor (preferably perindopril), angiotensin II type 1 receptor blocker Losartan, a tumour necrosis factor-alpha (TNFα) inhibitor, a source of mIGF-1, preferably mIGF-1, a compound for enhancing mIGF-1 expression, a compound for enhancing mIGF-1 activity, an antioxidant, an ion channel inhibitor, a protease inhibitor, L-arginine, a compound exhibiting a readthrough activity and/or inhibiting spliceosome assembly and/or splicing.

In one embodiment an individual is further provided with a functional dystrophin protein using a vector, preferably a viral vector, comprising a micro-mini-dystrophin gene. Most preferably, a recombinant adeno-associated viral (rAAV) vector is used. AAV is a single-stranded DNA parvovirus that is non-pathogenic and shows a helper-dependent life cycle. In contrast to other viruses (adenovirus, retrovirus, and herpes simplex virus), rAAV vectors have demonstrated to be very efficient in transducing mature skeletal muscle. Application of rAAV in classical DMD “gene addition” studies has been hindered by its restricted packaging limits (<5 kb). Therefore, rAAV is preferably applied for the efficient delivery of a much smaller micro- or mini-dystrophin gene. Administration of such micro- or mini-dystrophin gene results in the presence of an at least partially functional dystrophin protein. Reference is madeto¹⁸⁻²⁰.

Each constituent of a composition can be administered to an individual in any order. In one embodiment, each constituent is administered simultaneously (meaning that each constituent is administered within 10 hours, preferably within one hour). This is however not necessary. In one embodiment at least one adjunct compound is administered to an individual in need thereof before administration of an oligonucleotide. Alternatively, an oligonucleotide is administered to an individual in need thereof before administration of at least one adjunct compound.

### Use

In a further aspect, there is provided the use of a oligonucleotide or of a composition as defined herein for the manufacture of a medicament for preventing or treating Duchenne Muscular Dystrophy or Becker Muscular Dystrophy in an individual. Each feature of said use has earlier been defined herein.

A treatment in a use or in a method according to the invention is at least one week, at least one month, at least several months, at least one year, at least 2, 3, 4, 5, 6 years or more. Each molecule or oligonucleotide or equivalent thereof as defined herein for use according to the invention may be suitable for direct administration to a cell, tissue and/or an organ in vivo of individuals affected by or at risk of developing DMD or BMD, and may be administered directly in vivo, ex vivo or in vitro. The frequency of administration of an oligonucleotide, composition, compound or adjunct compound of the invention may depend on several parameters such as the age of the patient, the mutation of the patient, the number of molecules (i.e. dose), the formulation of said molecule. The frequency may be ranged between at least once in two weeks, or three weeks or four weeks or five weeks or a longer time period.

### Method

In a further aspect, there is provided a method for alleviating one or more symptom(s) of Duchenne Muscular Dystrophy or Becker Muscular Dystrophy in an individual or alleviate one or more characteristic(s) of a myogenic or muscle cell of said individual, the method comprising administering to said individual an oligonucleotide or a composition as defined herein.

There is further provided a method for enhancing, inducing or promoting skipping of an exon from a dystrophin pre-mRNA in a cell expressing said pre-mRNA in an individual suffering from Duchenne Muscular Dystrophy or Becker Muscular Dystrophy, the method comprising administering to said individual an oligonucleotide or a composition as defined herein. Further provided is a method for increasing the production of a functional dystrophin protein and/or decreasing the production of an aberrant dystrophin protein in a cell, said cell comprising a pre-mRNA of a dystrophin gene encoding an aberrant dystrophin protein, the method comprising providing said cell with an oligonucleotide or composition of the invention and allowing translation of mRNA produced from splicing of said pre-mRNA. In one embodiment, said method is performed in vitro, for instance using a cell culture. Preferably, said method is in vivo.

In this context, increasing the production of a functional dystrophin protein has been earlier defined herein.

Unless otherwise indicated each embodiment as described herein may be combined with another embodiment as described herein.

In this document and in its claims, the verb “to comprise” and its conjugations is used in its non-limiting sense to mean that items following the word are included, but items not specifically mentioned are not excluded. In addition the verb “to consist” may be replaced by “to consist essentially of meaning that a compound or adjunct compound as defined herein may comprise additional component(s) than the ones specifically identified, said additional component(s) not altering the unique characteristic of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “approximately” or “about” when used in association with a numerical value (approximately 10, about 10) preferably means that the value may be the given value of 10 more or less 1% of the value.

All patent and literature references cited in the present specification are hereby incorporated by reference in their entirety. Each embodiment as identified herein may be combined together unless otherwise indicated.

The invention is further explained in the following examples. These examples do not limit the scope of the invention, but merely serve to clarify the invention.

### BRIEF DESCRIPTION OF THE DRAWINGS

FIG. 1. In human control myotubes, PS220 and PS305 both targeting an identical sequence within exon 45, were directly compared for relative skipping efficiencies. PS220 reproducibly induced highest levels of exon 45 skipping (up to 73%), whereas with PS305 maximum exon 45 skipping levels of up to 46% were obtained. No exon 45 skipping was observed in non-treated cells. (M: DNA size marker; NT: non-treated cells)

FIG. 2. Graph showing relative exon 45 skipping levels of inosine-containing AONs as assessed by RT-PCR analysis. In human control myotubes, a series of new AONs, all targeting exon 45 and containing one inosine for guanosine substitution were tested for relative exon 45 skipping efficiencies when compared with PS220 and PS305 (see FIG. 1). All new inosine-containing AONs were effective, albeit at variable levels (between 4% and 25%). PS220 induced highest levels of exon 45 skipping (up to 72%), whereas with PS305 maximum exon 45 skipping levels of up to 63% were obtained. No exon 45 skipping was observed in non-treated cells. (M: DNA size marker; NT: non-treated cells).

### EXAMPLE 1

### Materials and Methods

AON design was based on (partly) overlapping open secondary structures of the target exon RNA as predicted by the m-fold program, on (partly) overlapping putative SR-protein binding sites as predicted by the ESE-finder software. AONs were synthesized by Prosensa Therapeutics B.V. (Leiden, Netherlands), and contain 2′-O-methyl RNA and full-length phosphorothioate (PS) backbones.

### Tissue Culturing, Transfection and RT-PCR Analysis

Myotube cultures derived from a healthy individual (“human control”) (examples 1, 3, and 4; exon 43, 50, 52 skipping) or a DMD patient carrying an exon 45 deletion (example 2; exon 46 skipping) were processed as described previously (Aartsma-Rus et al., Neuromuscul. Disord. 2002; 12: S71-77 and Hum Mol Genet 2003; 12(8): 907-14). For the screening of AONs, myotube cultures were transfected with 200 nM for each AON (PS220 and PS305). Transfection reagent UNIFectylin (Prosensa Therapeutics BV, Netherlands) was used, with 2 μl UNIFectylin per μg AON. Exon skipping efficiencies were determined by nested RT-PCR analysis using primers in the exons flanking the targeted exon 45. PCR fragments were isolated from agarose gels for sequence verification. For quantification, the PCR products were analyzed using the DNA 1000 LabChip Kit on the Agilent 2100 bioanalyzer (Agilent Technologies, USA).

### Results

DMD Exon 45 Skipping.

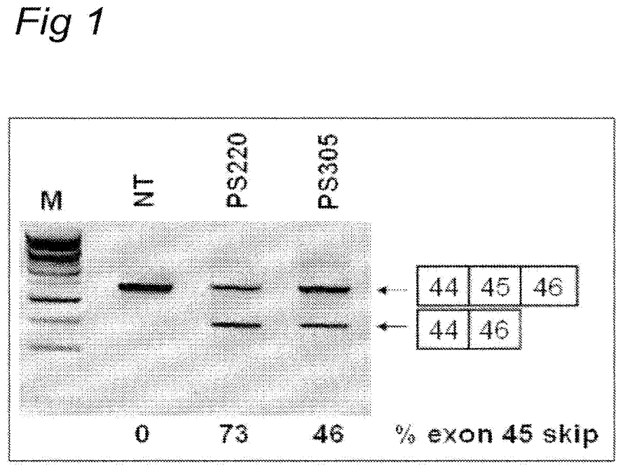

Two AONs, PS220 (SEQ ID NO: 76; 5′-UUUGCCGCUGCCCAAUGCCAUCCUG-3′) and PS305 (SEQ ID NO: 557; 5′-UUUGCCICUGCCCAAUGCCAUCCUG-3′) both targeting an identical sequence within exon 45, were directly compared for relative skipping efficiencies in healthy control myotube cultures. Subsequent RT-PCR and sequence analysis of isolated RNA demonstrated that both AONs were indeed capable of inducing exon 45 skipping. PS220, consisting a GCCGC stretch, reproducibly induced highest levels of exon 45 skipping (up to 73%), as shown in FIG. 1. However, PS305, which is identical to PS220 but containing an inosine for a G substitution at position 4 within that stretch is also effective and leading to exon 45 skipping levels of up to 46%. No exon 45 skipping was observed in non-treated cells (NT).

### EXAMPLE 2

### Materials and Methods

AON design was based on (partly) overlapping open secondary structures of the target exon 45 RNA as predicted by the m-fold program, on (partly) overlapping putative SR-protein binding sites as predicted by the ESE-finder software. AONs were synthesized by Prosensa Therapeutics B.V. (Leiden, Netherlands), and contain 2′-O-methyl RNA, full-length phosphorothioate (PS) backbones and one inosine for guanosine substitution.

### Tissue Culturing, Transfection and RT-PCR Analysis

Myotube cultures derived from a healthy individual (“human control”) were processed as described previously (Aartsma-Rus et al., Neuromuscul. Disord. 2002; 12: S71-77 and Hum Mol Genet 2003; 12(8): 907-14). For the screening of AONs, myotube cultures were transfected with 200 nM for each AON. Transfection reagent UNIFectylin (Prosensa Therapeutics BV, Netherlands) was used, with 2 μl UNIFectylin per 1.tg AON. Exon skipping efficiencies were determined by nested RT-PCR analysis using primers in the exons flanking the targeted exon 45. PCR fragments were isolated from agarose gels for sequence verification. For quantification, the PCR products were analyzed using the DNA 1000 Lab Chip Kit on the Agilent 2100 bioanalyzer (Agilent Technologies, USA).

### Results

DMD Exon 45 Skipping.

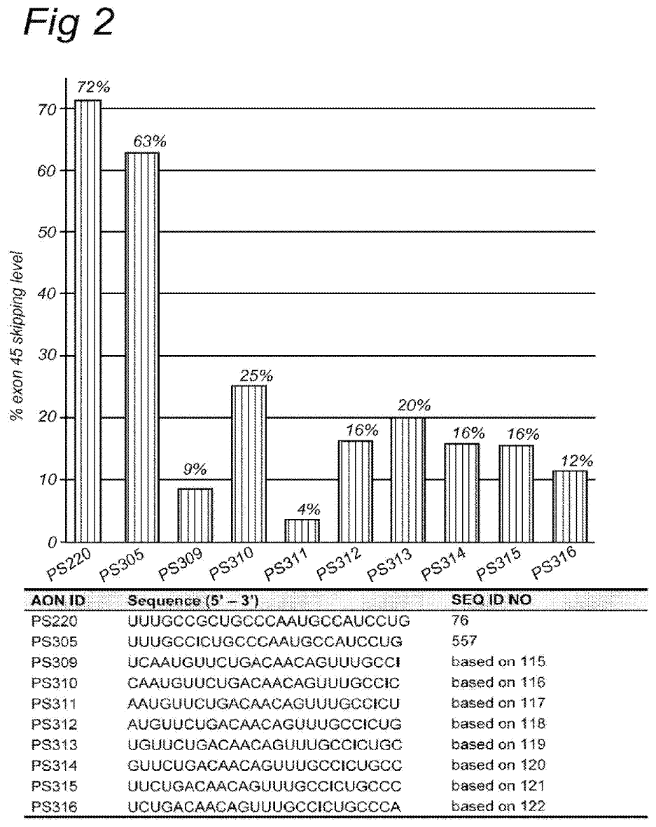

An additional series of AONs targeting exon 45 and containing one inosine-substitution were tested in healthy control myotube cultures for exon 45 skipping efficiencies, and directly compared to PS220 (without inosine; SEQ ID NO: 76) and PS305 (identical sequence as PS220 but with inosine substitution; SEQ ID NO: 557). Subsequent RT-PCR and sequence analysis of isolated RNA demonstrated that all new AONs (PS309 to PS316) were capable of inducing exon 45 skipping between 4% (PS311) and 25% (PS310) as shown in FIG. 2. When compared to PS220 and PS305, PS220 induced highest levels of exon 45 skipping (up to 72%). Of the new inosine-containing AONs PS305 was most effective, showing exon 45 skipping levels of up to 63%. No exon 45 skipping was observed in non-treated cells (NT).

### REFERENCES

1. Aartsma-Rus A, Janson A A, Kaman W E, et al. Therapeutic antisense-induced exon skipping in cultured muscle cells from six different DMD patients. Hum Mol Genet 2003; 12(8):907-14.
2. Aartsma-Rus A, Janson A A, Kaman W E, et al. Antisense-induced multi-exon skipping for Duchenne muscular dystrophy makes more sense. Am J Hum Genet 2004; 74(1):83-92.
3. Alter J, Lou F, Rabinowitz A, et al. Systemic delivery of morpholino oligonucleotide restores dystrophin expression bodywide and improves dystrophic pathology. Nat Med 2006; 12(2):175-7.
4. Goyenvalle A, Vulin A, Fougerousse F, et al. Rescue of dystrophic muscle through U7 snRNA-mediated exon skipping. Science 2004; 306(5702):1796-9.
5. Lu Q L, Mann C J, Lou F, et al. Functional amounts of dystrophin produced by skipping the mutated exon in the mdx dystrophic mouse. Nat Med 2003; 6:6.
6. Lu Q L, Rabinowitz A, Chen Y C, et al. Systemic delivery of antisense oligoribonucleotide restores dystrophin expression in body-wide skeletal muscles. Proc Natl Acad Sci USA 2005; 102(1):198-203.
7. McClorey G, Fall A M, Moulton H M, et al. Induced dystrophin exon skipping in human muscle explants. Neuromuscul Disord 2006; 16(9-10):583-90.
8. McClorey G, Moulton H M, Iversen P L, et al. Antisense oligonucleotide-induced exon skipping restores dystrophin expression in vitro in a canine model of DMD. Gene Ther 2006; 13(19):1373-81.
9. Pramono Z A, Takeshima Y, Alimsardj ono H, Ishii A, Takeda S, Matsuo M. Induction of exon skipping of the dystrophin transcript in lymphoblastoid cells by transfecting an antisense oligodeoxynucleotide complementary to an exon recognition sequence. Biochem Biophys Res Commun 1996; 226(2):445-9.
10. Takeshima Y, Yagi M, Wada H, et al. Intravenous infusion of an antisense oligonucleotide results in exon skipping in muscle dystrophin mRNA of Duchenne muscular dystrophy. Pediatr Res 2006; 59(5):690-4.
11. van Deutekom J C, Bremmer-Bout M, Janson A A, et al. Antisense-induced exon skipping restores dystrophin expression in DMD patient derived muscle cells. Hum Mol Genet 2001; 10(15):1547-54.
12. Aartsma-Rus A, Bremmer-Bout M, Janson A, den Dunnen J, van Ommen G, van Deutekom J. Targeted exon skipping as a potential gene correction therapy for Duchenne muscular dystrophy. Neuromuscul Disord 2002; 12 Suppl: S71-S77.
13. Aartsma-Rus A, De Winter C L, Janson A A, et al. Functional analysis of 114 exon-internal AONs for targeted DMD exon skipping: indication for steric hindrance of S R protein binding sites. Oligonucleotides 2005; 15(4):284-97.
14. Aartsma-Rus A, Janson A A, Heemskerk J A, C L de Winter, G J Van Ommen, J C Van Deutekom. Therapeutic Modulation of DMD Splicing by Blocking Exonic Splicing Enhancer Sites with Antisense Oligonucleotides. Annals of the New York Academy of Sciences 2006; 1082:74-6.
15. Aartsma-Rus A, Kaman W E, Weij R, den Dunnen J T, van Ommen G J, van Deutekom J C. Exploring the frontiers of therapeutic exon skipping for Duchenne muscular dystrophy by double targeting within one or multiple exons. Mol Ther 2006; 14(3):401-7.
16. Welch E M, Barton E R, Zhuo J, et al. PTC124 targets genetic disorders caused by nonsense mutations. Nature 2007; 447(7140):87-91.
17. Hirawat S, Welch E M, Elfring G L, et al. Safety, tolerability, and pharmacokinetics of PTC124, a nonaminoglycoside nonsense mutation suppressor, following single- and multiple-dose administration to healthy male and female adult volunteers. Journal of clinical pharmacology 2007; 47(4):430-44.
18. Wang B, Li J, Xiao X. Adeno-associated virus vector carrying human minidystrophin genes effectively ameliorates muscular dystrophy in mdx mouse model. Proc Natl Acad Sci USA 2000; 97(25):13714-9.
19. Fabb S A, Wells D J, Serpente P, Dickson G. Adeno-associated virus vector gene transfer and sarcolemmal expression of a 144 kDa micro-dystrophin effectively restores the dystrophin-associated protein complex and inhibits myofibre degeneration in nude/mdx mice. Hum Mol Genet 2002; 11(7):733-41.
20. Wang Z, Kuhr C S, Allen J M, et al. Sustained AAV-mediated dystrophin expression in a canine model of Duchenne muscular dystrophy with a brief course of immunosuppression. Mol Ther 2007; 15(6):1160-6.
21. Manzur A Y, Kuntzer T, Pike M, Swan A. Glucocorticoid corticosteroids for Duchenne muscular dystrophy. Cochrane Database Syst Rev 2004; 2.
22. Duboc D, Meune C, Pierre B, et al. Perindopril preventive treatment on mortality in Duchenne muscular dystrophy: 10 years' follow-up. American heart journal 2007; 154(3):596-602.
23. Cohn R D, van Erp C, Habashi J P, et al. Angiotensin II type 1 receptor blockade attenuates TGF-beta-induced failure of muscle regeneration in multiple myopathic states. Nat Med 2007; 13(2):204-10.
24. Grounds M D, Torrisi J. Anti-TNFalpha (Remicade) therapy protects dystrophic skeletal muscle from necrosis. Faseb J 2004; 18(6):676-82.
25. Hodgetts S, Radley H, Davies M, Grounds M D. Reduced necrosis of dystrophic muscle by depletion of host neutrophils, or blocking TNFalpha function with Etanercept in mdx mice. Neuromuscul Disord 2006; 16(9-10):591-602.
26. Pierno S, Nico B, Burdi R, et al. Role of tumour necrosis factor alpha, but not of cyclo-oxygenase-2-derived eicosanoids, on functional and morphological indices of dystrophic progression in mdx mice: a pharmacological approach. Neuropathology and applied neurobiology 2007; 33(3):344-59.
27. Musaro A, McCullagh K, Paul A, et al. Localized Igf-1 transgene expression sustains hypertrophy and regeneration in senescent skeletal muscle. Nat Genet 2001; 27(2):195-200.
28. Barton E R, Morris L, Musaro A, Rosenthal N, Sweeney H L. Muscle-specific expression of insulin-like growth factor I counters muscle decline in mdx mice. J Cell Biol 2002; 157(1):137-48.
29. Disatnik M H, Dhawan J, Yu Y, et al. Evidence of oxidative stress in mdx mouse muscle: studies of the pre-necrotic state. J Neurol Sci 1998; 161(1):77-84.
30. Nelson S K, Bose S K, Grunwald G K, Myhill P, McCord J M. The induction of human superoxide dismutase and catalase in vivo: a fundamentally new approach to antioxidant therapy. Free radical biology & medicine 2006; 40(2):341-7.
31. Hart P E, Lodi R, Rajagopalan B, et al. Antioxidant treatment of patients with Friedreich ataxia: four-year follow-up. Archives of neurology 2005; 62(4):621-6.
32. Rolland J F, De Luca A, Burdi R, Andreetta F, Confalonieri P, Conte Camerino D. Overactivity of exercise-sensitive cation channels and their impaired modulation by IGF-1 in mdx native muscle fibers: beneficial effect of pentoxifylline. Neurobiol Dis 2006; 24(3):466-74.
33. Whitehead N P, Streamer M, Lusambili L I, Sachs F, Allen D G. Streptomycin reduces stretch-induced membrane permeability in muscles from mdx mice. Neuromuscul Disord 2006; 16(12):845-54.
34. Badalamente M A, Stracher A. Delay of muscle degeneration and necrosis in mdx mice by calpain inhibition. Muscle Nerve 2000; 23(1):106-11.
35. Burdi R, Didonna M P, Pignol B, et al. First evaluation of the potential effectiveness in muscular dystrophy of a novel chimeric compound, BN 82270, acting as calpain-inhibitor and anti-oxidant. Neuromuscul Disord 2006; 16(4):237-48.
36. Bonuccelli G, Sotgia F, Schubert W, et al. Proteasome inhibitor (MG-132) treatment of mdx mice rescues the expression and membrane localization of dystrophin and dystrophin-associated proteins. Am J Pathol 2003; 163(4):1663-75.
37. Voisin V, Sebrie C, Matecki S, et al. L-arginine improves dystrophic phenotype in mdx mice. Neurobiol Dis 2005; 20(1):123-30.
38. Soret J, Bakkour N, Maire S, et al. Selective modification of alternative splicing by indole derivatives that target serine-arginine-rich protein splicing factors. Proc Natl Acad Sci USA 2005; 102(24):8764-9.
39. Mann C J, Honeyman K, McClorey G, Fletcher S, Wilton S D. Improved antisense oligonucleotide induced exon skipping in the mdx mouse model of muscular dystrophy. J Gene Med 2002; 4(6):644-54.
40. Graham I R, Hill V J, Manoharan M, Inamati G B, Dickson G. Towards a therapeutic inhibition of dystrophin exon 23 splicing in mdx mouse muscle induced by antisense oligoribonucleotides (splicomers): target sequence optimisation using oligonucleotide arrays. J Gene Med 2004; 6(10):1149-58.
41. Mathews D H, Sabina J, Zuker M, Turner D H. Expanded sequence dependence of thermodynamic parameters improves prediction of RNA secondary structure. J Mol Biol 1999; 288(5):911-40.
42. Cartegni L, Chew S L, Krainer A R. Listening to silence and understanding nonsense: exonic mutations that affect splicing. Nat Rev Genet 2002; 3(4):285-98.
43. Cartegni L, Wang J, Zhu Z, Zhang M Q, Krainer A R. ESEfinder: A web resource to identify exonic splicing enhancers. Nucleic Acids Res 2003; 31(13):3568-71.
44. Braasch D A, Corey D R. Locked nucleic acid (LNA): fine-tuning the recognition of DNA and RNA. Chem Biol 2001; 8(1):1-7.
45. Braasch D A, Corey D R. Novel antisense and peptide nucleic acid strategies for controlling gene expression. Biochemistry 2002; 41(14):4503-10.
46. Elayadi A N, Corey D R. Application of PNA and LNA oligomers to chemotherapy. Curr Opin Investig Drugs 2001; 2(4):558-61.
47. Larsen H J, Bentin T, Nielsen P E. Antisense properties of peptide nucleic acid. Biochim Biophys Acta 1999; 1489(1):159-66.
48. Summerton J. Morpholino antisense oligomers: the case for an RNase H-independent structural type. Biochim Biophys Acta 1999; 1489(1):141-58.
49. Summerton J, Weller D. Morpholino antisense oligomers: design, preparation, and properties. Antisense Nucleic Acid Drug Dev 1997; 7(3):187-95.
50. Wahlestedt C, Salmi P, Good L, et al. Potent and nontoxic antisense oligonucleotides containing locked nucleic acids. Proc Natl Acad Sci USA 2000; 97(10):5633-8.
51. De Angelis F G, Sthandier O, Berarducci B, et al. Chimeric snRNA molecules carrying antisense sequences against the splice junctions of exon 51 of the dystrophin pre-mRNA induce exon skipping and restoration of a dystrophin synthesis in Delta 48-50 DMD cells. Proc Natl Acad Sci USA 2002; 99(14):9456-61.
52. Denti M A, Rosa A, D′Antona G, et al. Chimeric adeno-associated virus/antisense U1 small nuclear RNA effectively rescues dystrophin synthesis and muscle function by local treatment of mdx mice. Hum Gene Ther 2006; 17(5):565-74.
53. Gorman L, Suter D, Emerick V, Schumperli D, Kole R. Stable alteration of pre-mRNA splicing patterns by modified U7 small nuclear RNAs. Proc Natl Acad Sci USA 1998; 95(9):4929-34.
54. Suter D, Tomasini R, Reber U, Gorman L, Kole R, Schumperli D. Double-target antisense U7 snRNAs promote efficient skipping of an aberrant exon in three human beta-thalassemic mutations. Hum Mol Genet 1999; 8(13):2415-23.
55. Wagner K R, Hamed S, Hadley D W, et al. Gentamicin treatment of Duchenne and Becker muscular dystrophy due to nonsense mutations. Ann Neurol 2001; 49(6):706-11.
56. Aartsma-Rus A et al, (2006), Entries in the leiden Duchenne Muscular Dystrophy mutation database: an overview of mutation types and paradoxical cases that confirm the reading-frame rule, Muscle Nerve, 34: 135-144.
57. Hodgetts S., et al, (2006), Neuromuscular Disorders, 16: 591-602.
58. Manzur A Y et al, (2008), Glucocorticoid corticosteroids for Duchenne muscular dystrophy (review), Wiley publishers, The Cochrane collaboration.
59. Yokota T. et al., Mar. 13, 2009, e-publication: Efficacy of systemic morpholino exon-skipping in duchennes dystrophy dogs. Ann. Neurol. 2009
60. Dorn and Kippenberger, Curr Opin Mol Ther 2008 10(1) 10-20
61. Cheng and Van Dyke, Gene. 1997 Sep. 15; 197(1-2):253-60
62. Macaya et al., Biochemistry. 1995 4; 34(13):4478-92.
63. Suzuki et al., Eur J Biochem. 1999, 260(3):855-6
64. Howard et al., Ann Neurol 2004 55(3): 422-6;
65. Nudelman et al., 2006, Bioorg Med Chem Lett 16(24), 6310-5

Sequence listing
DMD gene amino acid sequence
SEQ ID NO 1:
MLWWEEVEDCYEREDVQKKTFTKWVNAQFSKFGKQHIENLFSDLQDGRRLLDLLEGL
TCQKLPKEKGSTRVHALNNVNKALRVLQNNNVDLVNIGSTDIVDGNHKLTLGLIWNIIL
HWQVKNVMKNIMAGLQQTNSEKILLSWVRQSTRNYPQVNVINFITSWSDGLALNALIH
SHRPDLFDWNSVVCQQSATQRLEHAFNIARYQLGIEKLLDPEDVDTTYPDKKSILMYIT
SLFQVLPQQVSIEAIQEVEMLPRPPKVTKEEHFQLHHQMHYSQQITVSLAQGYERTSSP
KPRFKSYAYTQAAYVTTSDPTRSPFPSQHLEAPEDKSFGSSLMESEVNLDRYQTALEEV
LSWLLSAEDTLQAQGEISNDVEVVKDQFHTHEGYMMDLTAHQGRVGNILQLGSKLIGT
GKLSEDEETEVQEQMNLLNSRWECLRVASMEKQSNLHRVLMDLQNQKLKELNDWLT
KTEERTRKMEEEPLGPDLEDLKRQVQQHKVLQEDLEQEQVRVNSLTHMVVVVDESSG
DHATAALEEQLKVLGDRWANICRWTEDRWVLLQDILLKWQRLTEEQCLFSAWLSEKE
DAVNKIHTTGFKDQNEMLSSLQKLAVLKADLEKKKQSMGKLYSLKQDLLSTLKNKSVT
QKTEAWLDNFARCWDNLVQKLEKSTAQISQAVTTTQPSLTQTTVMETVTTVTTREQILV
KHAQEELPPPPPQKKRQITVDSEIRKRLDVDITELHSWITRSEAVLQSPEFAIFRKEGNF
SDLKEKVNAIEREKAEKFRKLQDASRSAQALVEQMVNEGVNADSIKQASEQLNSRWIE
FCQLLSERLNWLEYQNNIIAFYNQLQQLEQMTTTAENWLKIQPTTPSEPTAIKSQLKIC
KDEVNRLSGLQPQIERLKIQSIALKEKGQGPMFLDADFVAFTNHFKQVFSDVQAREKEL
QTIFDTLPPMRYQETMSAIRTWVQQSETKLSIPQLSVTDYEIMEQRLGELQALQSSLQE
QQSGLYYLSTTVKEMSKKAPSEISRKYQSEFEEIEGRWKKLSSQLVEHCQKLEEQMNK
LRKIQNHIQTLKKWMAEVDVFLKEEWPALGDSEILKKQLKQCRLLVSDIQTIQPSLNSV
NEGGQKIKNEAEPEFASRLETELKELNTQWDHMCQQVYARKEALKGGLEKTVSLQKD
LSEMHEWMTQAEEEYLERDFEYKTPDELQKAVEEMKRAKEEAQQKEAKVKLLTESV
NSVIAQAPPVAQEALKKELETLTTNYQWLCTRLNGKCKTLEEVWACWHELLSYLEKAN
KWLNEVEFKLKTTENIPGGAEEISEVLDSLENLMRHSEDNPNQIRILAQTLTDGGVMD
ELINEELETFNSRWRELHEEAVRRQKLLEQSIQSAQETEKSLHLIQESLTFIDKQLAAYI
ADKVDAAQMPQEAQKIQSDLTSHEISLEEMKKHNQGKEAAQRVLSQIDVAQKKLQDVS
MKFRLFQKPANFEQRLQESKMILDEVKMHLPALETKSVEQEVVQSQLNHCVNLYKSLS
EVKSEVEMVIKTGRQIVQKKQTENPKELDERVTALKLHYNELGAKVTERKQQLEKCLK
LSRKMRKEMNVLTEWLAATDMELTKRSAVEGMPSNLDSEVAWGKATQKEIEKQKVH
LKSITEVGEALKTVLGKKETLVEDKLSLLNSNWIAVTSRAEEWLNLLLEYQKHMETFD
QNVDHITKWIIQADTLLDESEKKKPQQKEDVLKRLKAELNDIRPKVDSTRDQAANLMA
NRGDHCRKLVE PQISELNHRFAAISHRIKTGKASIPLKELEQFNSDIQKLLEPLEAEIQQ
GVNLKEEDFNKDMNEDNEGTVKELLQRGDNLQQRITDERKREEIKIKQQLLQTKHNA
LKDLRSQRRKKALEISHQWYQYKRQADDLLKCLDDIEKKLASLPEPRDERKIKEIDREL
QKKKEELNAVRRQAEGLSEDGAAMAVEPTQIQLSKRWREIESKFAQFRRLNFAQIHTV
REETMMVMTEDMPLEISYVPSTYLTEITHVSQALLEVEQLLNAPDLCAKDFEDLFKQE
ESLKNIKDSLQQSSGRIDIIHSKKTAALQSATPVERVKLQEALSQLDFQWEKVNKMYKD
RQGRFDRSVEKWRRFHYDIKIFNQWLTEAEQFLRKTQIPENWEHAKYKWYLKELQDGI
CQRQTVVRTLNATGEEIIQQSSKTDASILQEKLGSLNLRWQEVCKQLSDRKKRLEEQKN
ILSEFQRDLNEFVLWLEEADNIASIPLEPGKEQQLKEKLEQVKLLVEELPLRQGILKQL
NETGGPVLVSAPISPEEQDKLENKLKQTNLQWIKVSRALPEKQGEIEAQIKDLGQLEKK
LEDLEEQLNHLLLWLSPIRNQLEIYNQPNQEGPFDVQETEIAVQAKQPDVEEILSKGQH
LYKEKPATQPVKRKLEDLSSEWKAVNRLLQELRAKQPDLAPGLTTIGASPTQTVTLVTQ
PVVTKETAISKLEMPSSLMLEVPALADFNRAWTELTDWLSLLDQVIKSQRVMVGDLEDI
NEMIIKQKATMQDLEQRRPQLEELITAAQNLKNKTSNQEARTIITDRIERIQNQWDEVQ
EHLQNRRQQLNEMLKDSTQWLEAKEEAEQVLGQARAKLESWKEGPYTVDAIQKKITE
TKQLAKDLRQWQTNVDVANDLALKLLRDYSADDTRKVHMITENINASWRSIHKRVSER
EAALEETHRLLQQFPLDLEKFLAWLTEAETTANVLQDATRKERLLEDSKGVKELMKQ
WQDLQGEIEAHTDVYHNLDENSQKILRSLEGSDDAVLLQRRLDNMNFKWSELRKKSL
NIRSHLEASSDQWKRLHLSLQELLVWLQLKDDELSRQAPIGGDFPAVQKQNDVHRAFK
RELKTKEPVIMSTLETVRIFLTEQPLEGLEKLYQEPRELPPEERAQNVTRLLRKQAEEV
NTEWEKLNLHSADWQRKIDETLERLQELQEATDELDLKLRQAEVIKGSWQPVGDLLID
SLQDHLEKVKALRGEIAPLKENVSHVNDLARQLTTLGIQLSPYNLSTLEDLNTRWKLLQ
VAVEDRVRQLHEAHRDFGPASQHFLSTSVQGPWERAISPNKVPYYINHETQTTCWDHP
KMTELYQSLADLNNVRFSAYRTAMKLRRLQKALCLDLLSLSAACDALDQHNLKQNDQ
PMDILQIINCLTTIYDRLEQEHNNLVNVPLCVDMCLNWLLNVYDTGRTGRIRVLSFKTG
IISLCKAHLEDKYRYLFKQVASSTGFCDQRRLGLLLHDSIQIPRQLGEVASFGGSNIEPSV
RSCFQFANNKPEIEAALFLDWMRLEPQSMVWLPVLHRVAAAETAKHQAKCNICKECPI
IGFRYRSLKHFNYDICQSCFFSGRVAKGHKMHYPMVEYCTPTTSGEDVRDFAKVLKNK
FRTKRYFAKHPRMGYLPVQTVLEGDNMETPVTLINFWPVDSAPASSPQLSHDDTHSRI
EHYASRLAEMENSNGSYLNDSISPNESIDDEHLLIQHYCQSLNQDSPLSQPRSPAQILIS
LESEERGELERILADLEEENRNLQAEYDRLKQQHEHKGLSPLPSPPEMMPTSPQSPRD
AELIAEAKLLRQHKGRLEARMQILEDHNKQLESQLHRLRQLLEQPQAEAKVNGTTVSS
PSTSLQRSDSSQPMLLRVVGSQTSDSMGEEDLLSPPQDTSTGLEEVMEQLNNSFPSSRG
RNTPGKPMREDTM
DMD Gene Exon 51

| | | | |
|---|---|---|---|
| SEQ ID | GUACCUCCAACAUCAAGGAAGAUGG | SEQ ID | GAGAUGGCAGUUUCCUUAGUAACCA |
| NO 2 | | NO 39 | |
| SEQ ID | UACCUCCAACAUCAAGGAAGAUGGC | SEQ ID | AGAUGGCAGUUUCCUUAGUAACCAC |
| NO 3 | | NO 40 | |
| SEQ ID | ACCUCCAACAUCAAGGAAGAUGGCA | SEQ ID | GAUGGCAGUUUCCUUAGUAACCACA |
| NO 4 | | NO 41 | |
| SEQ ID | CCUCCAACAUCAAGGAAGAUGGCAU | SEQ ID | AUGGCAGUUUCCUUAGUAACCACAG |
| NO 5 | | NO 42 | |
| SEQ ID | CUCCAACAUCAAGGAAGAUGGCAUU | SEQ ID | UGGCAGUUUCCUUAGUAACCACAGG |
| NO 6 | | NO 43 | |
| SEQ ID | UCCAACAUCAAGGAAGAUGGCAUUU | SEQ ID | GGCAGUUUCCUUAGUAACCACAGGU |
| NO 7 | | NO 44 | |
| SEQ ID | CCAACAUCAAGGAAGAUGGCAUUUC | SEQ ID | GCAGUUUCCUUAGUAACCACAGGUU |
| NO 8 | | NO 45 | |
| SEQ ID | CAACAUCAAGGAAGAUGGCAUUUCU | SEQ ID | CAGUUUCCUUAGUAACCACAGGUUG |
| NO 9 | | NO 46 | |
| SEQ ID | AACAUCAAGGAAGAUGGCAUUUCUA | SEQ ID | AGUUUCCUUAGUAACCACAGGUUGU |
| NO 10 | | NO 47 | |
| SEQ ID | ACAUCAAGGAAGAUGGCAUUUCUAG | SEQ ID | GUUUCCUUAGUAACCACAGGUUGUG |
| NO 11 | | NO 48 | |
| SEQ ID | CAUCAAGGAAGAUGGCAUUUCUAGU | SEQ ID | UUUCCUUAGUAACCACAGGUUGUGU |
| NO 12 | | NO 49 | |
| SEQ ID | AUCAAGGAAGAUGGCAUUUCUAGUU | SEQ ID | UUCCUUAGUAACCACAGGUUGUGUC |
| NO 13 | | NO 50 | |
| SEQ ID | UCAAGGAAGAUGGCAUUUCUAGUUU | SEQ ID | UCCUUAGUAACCACAGGUUGUGUCA |
| NO 14 | | NO 51 | |
| SEQ ID | CAAGGAAGAUGGCAUUUCUAGUUUG | SEQ ID | CCUUAGUAACCACAGGUUGUGUCAC |
| NO 15 | | NO 52 | |
| SEQ ID | AAGGAAGAUGGCAUUUCUAGUUUGG | SEQ ID | CUUAGUAACCACAGGUUGUGUCACC |
| NO 16 | | NO 53 | |
| SEQ ID | AGGAAGAUGGCAUUUCUAGUUUGGA | SEQ ID | UUAGUAACCACAGGUUGUGUCACCA |
| NO 17 | | NO 54 | |
| SEQ ID | GGAAGAUGGCAUUUCUAGUUUGGAG | SEQTD | UAGUAACCACAGGUUGUGUCACCAG |
| NO 18 | | NO 55 | |
| SEQ ID | GAAGAUGGCAUUUCUAGUUUGGAGA | SEQ ID | AGUAACCACAGGUUGUGUCACCAGA |
| NO 19 | | NO 56 | |
| SEQ ID | AAGAUGGCAUUUCUAGUUUGGAGAU | SEQ ID | GUAACCACAGGUUGUGUCACCAGAG |
| NO 20 | | NO 57 | |
| SEQ ID | AGAUGGCAUUUCUAGUUUGGAGAUG | SEQ ID | UAACCACAGGUUGUGUCACCAGAGU |
| NO 21 | | NO 58 | |
| SEQ ID | GAUGGCAUUUCUAGUUUGGAGAUGG | SEQ ID | AACCACAGGUUGUGUCACCAGAGUA |
| NO 22 | | NO 59 | |
| SEQ ID | AUGGCAUUUCUAGUUUGGAGAUGGC | SEQ ID | ACCACAGGUUGUGUCACCAGAGUAA |
| NO 23 | | NO 60 | |
| SEQ ID | UGGCAUUUCUAGUUUGGAGAUGGCA | SEQ ID | CCACAGGUUGUGUCACCAGAGUAAC |
| NO 24 | | NO 61 | |
| SEQ ID | GGCAUUUCUAGUUUGGAGAUGGCAG | SEQ ID | CACAGGUUGUGUCACCAGAGUAACA |
| NO 25 | | NO 62 | |
| SEQ ID | GCAUUUCUAGUUUGGAGAUGGCAGU | SEQ ID | ACAGGUUGUGUCACCAGAGUAACAG |
| NO 26 | | NO 63 | |
| SEQ ID | CAUUUCUAGUUUGGAGAUGGCAGUU | SEQ ID | CAGGUUGUGUCACCAGAGUAACAGU |
| NO 27 | | NO 64 | |
| SEQ ID | AUUUCUAGUUUGGAGAUGGCAGUUU | SEQ ID | AGGUUGUGUCACCAGAGUAACAGUC |
| NO 28 | | NO 65 | |
| SEQ ID | UUUCUAGUUUGGAGAUGGCAGUUUC | SEQ ID | GGUUGUGUCACCAGAGUAACAGUCU |
| NO 29 | | NO 66 | |
| SEQ ID | UUCUAGUUUGGAGAUGGCAGUUUCC | SEQ ID | GUUGUGUCACCAGAGUAACAGUCUG |
| NO 30 | | NO 67 | |
| SEQ ID | UCUAGUUUGGAGAUGGCAGUUUCCU | SEQ ID | UUGUGUCACCAGAGUAACAGUCUGA |
| NO 31 | | NO 68 | |
| SEQ ID | CUAGUUUGGAGAUGGCAGUUUCCUU | SEQ ID | UGUGUCACCAGAGUAACAGUCUGAG |
| NO 32 | | NO 69 | |
| SEQ ID | UAGUUUGGAGAUGGCAGUUUCCUUA | SEQ ID | GUGUCACCAGAGUAACAGUCUGAGU |
| NO 33 | | NO 70 | |
| SEQ ID | AGUUUGGAGAUGGCAGUUUCCUUAG | SEQ ID | UGUCACCAGAGUAACAGUCUGAGUA |
| NO 34 | | NO 71 | |
| SEQ ID | GUUUGGAGAUGGCAGUUUCCUUAGU | SEQ ID | GUCACCAGAGUAACAGUCUGAGUAG |
| NO 35 | | NO 72 | |
| SEQ ID | UUUGGAGAUGGCAGUUUCCUUAGUA | SEQ ID | UCACCAGAGUAACAGUCUGAGUAGG |
| NO 36 | | NO 73 | |
| SEQ ID | UUGGAGAUGGCAGUUUCCUUAGUAA | SEQ ID | CACCAGAGUAACAGUCUGAGUAGGA |
| NO 37 | | NO 74 | |
| SEQ ID | UGGAGAUGGCAGUUUCCUUAGUAAC | SEQ ID | ACCAGAGUAACAGUCUGAGUAGGAG |
| NO 38 | | NO 75 | |
| SEQ ID | UCAAGGAAGAUGGCAUUUCU | SEQ ID | UCAAGGAAGAUGGCAUIUCU |
| NO 539 | | NO 548 | |
| SEQ ID | UCAAIGAAGAUGGCAUUUCU | SEQ ID | UCAAGGAAGAUGGCAUUICU |
| NO 540 | | NO 549 | |
| SEQ ID | UCAAGIAAGAUGGCAUUUCU | SEQ ID | UCAAGGAAGAUGGCAUUUCI |
| NO 541 | | NO 550 | |
| SEQ ID | UCAAGGAAIAUGGCAUUUCU | SEQ ID | UCIAGGAAGAUGGCAUUUCU |
| NO 542 | | NO 551 | |
| SEQ ID | UCAAGGAAGAUIGCAUUUCU | SEQ ID | UCAIGGAAGAUGGCAUUUCU |
| NO 543 | | NO 552 | |
| SEQ ID | UCAAGGAAGAUGICAUUUCU | SEQ ID | UCAAGGIAGAUGGCAUUUCU |
| NO 544 | | NO 553 | |
| SEQ ID | ICAAGGAAGAUGGCAUUUCU | SEQ ID | UCAAGGAIGAUGGCAUUUCU |
| NO 545 | | NO 554 | |
| SEQ ID | UCAAGGAAGAIGGCAUUUCU | SEQ ID | UCAAGGAAGIUGGCAUUUCU |
| NO 546 | | NO 555 | |
| SEQ ID | UCAAGGAAGAUGGCAIUUCU | SEQ ID | UCAAGGAAGAUGGCIUUUCU |
| NO 547 | | NO 556 | |

DMD Gene Exon 45

| | | | |
|---|---|---|---|
| SEQ ID | UUUGCCGCUGCCCAAUGCCAUCCUG | SEQ ID | GUUGCAUUCAAUGUUCUGACAACAG |
| NO 76 | | NO 109 | |
| PS220 | | | |
| SEQ ID | AUUCAAUGUUCUGACAACAGUUUGC | SEQ ID | UUGCAUUCAAUGUUCUGACAACAGU |
| NO 77 | | NO 110 | |
| SEQ ID | CCAGUUGCAUUCAAUGUUCUGACAA | SEQ ID | UGCAUUCAAUGUUCUGACAACAGUU |
| NO 78 | | NO 111 | |
| SEQ ID | CAGUUGCAUUCAAUGUUCUGAC | SEQ ID | GCAUUCAAUGUUCUGACAACAGUUU |
| NO 79 | | NO 112 | |
| SEQ ID | AGUUGCAUUCAAUGUUCUGA | SEQ ID | CAUUCAAUGUUCUGACAACAGUUUG |
| NO 80 | | NO 113 | |
| SEQ ID | GAUUGCUGAAUUAUUUCUUCC | SEQ ID | AUUCAAUGUUCUGACAACAGUUUGC |
| NO 81 | | NO 114 | |
| SEQ ID | GAUUGCUGAAUUAUUUCUUCCCCAG | SEQ ID | UCAAUGUUCUGACAACAGUUUGCCG |
| NO 82 | | NO 115 | |
| SEQ ID | AUUGCUGAAUUAUUUCUUCCCCAGU | SEQ ID | CAAUGUUCUGACAACAGUUUGCCGC |
| NO 83 | | NO 116 | |
| SEQ ID | UUGCUGAAUUAUUUCUUCCCCAGUU | SEQ ID | AAUGUUCUGACAACAGUUUGCCGCU |
| NO 84 | | NO 117 | |
| SEQ ID | UGCUGAAUUAUUUCUUCCCCAGUUG | SEQ ID | AUGUUCUGACAACAGUUUGCCGCUG |
| NO 85 | | NO 118 | |
| SEQ ID | GCUGAAUUAUUUCUUCCCCAGUUGC | SEQ ID | UGUUCUGACAACAGUUUGCCGCUGC |
| NO 86 | | NO 119 | |
| SEQ ID | CUGAAUUAUUUCUUCCCCAGUUGCA | SEQ ID | GUUCUGACAACAGUUUGCCGCUGCC |
| NO 87 | | NO 120 | |
| SEQ ID | UGAAUUAUUUCUUCCCCAGUUGCAU | SEQ ID | UUCUGACAACAGUUUGCCGCUGCCC |
| NO 88 | | NO 121 | |
| SEQ ID | GAAUUAUUUCUUCCCCAGUUGCAUU | SEQ ID | UCUGACAACAGUUUGCCGCUGCCCA |
| NO 89 | | NO 122 | |
| SEQ ID | AAUUAUUUCUUCCCCAGUUGCAUUC | SEQ ID | CUGACAACAGUUUGCCGCUGCCCAA |
| NO 90 | | NO 123 | |
| SEQ ID | AUUAUUUCUUCCCCAGUUGCAUUCA | SEQ ID | UGACAACAGUUUGCCGCUGCCCAAU |
| NO 91 | | NO 124 | |
| SEQ ID | UUAUUUCUUCCCCAGUUGCAUUCAA | SEQ ID | GACAACAGUUUGCCGCUGCCCAAUG |
| NO 92 | | NO 125 | |
| SEQ ID | UAUUUCUUCCCCAGUUGCAUUCAAU | SEQ ID | ACAACAGUUUGCCGCUGCCCAAUGC |
| NO 93 | | NO 126 | |
| SEQ ID | AUUUCUUCCCCAGUUGCAUUCAAUG | SEQ ID | CAACAGUUUGCCGCUGCCCAAUGCC |
| NO 94 | | NO 127 | |
| SEQ ID | UUUCUUCCCCAGUUGCAUUCAAUGU | SEQ ID | AACAGUUUGCCGCUGCCCAAUGCCA |
| NO 95 | | NO 128 | |
| SEQ ID | UUCUUCCCCAGUUGCAUUCAAUGUU | SEQ ID | ACAGUUUGCCGCUGCCCAAUGCCAU |
| NO 96 | | NO 129 | |
| SEQ ID | UCUUCCCCAGUUGCAUUCAAUGUUC | SEQ ID | CAGUUUGCCGCUGCCCAAUGCCAUC |
| NO 97 | | NO 130 | |
| SEQ ID | CUUCCCCAGUUGCAUUCAAUGUUCU | SEQ ID | AGUUUGCCGCUGCCCAAUGCCAUCC |
| NO 98 | | NO 131 | |
| SEQ ID | UUCCCCAGUUGCAUUCAAUGUUCUG | SEQ ID | GUUUGCCGCUGCCCAAUGCCAUCCU |
| NO 99 | | NO 132 | |
| SEQ ID | UCCCCAGUUGCAUUCAAUGUUCUGA | SEQ ID | UUUGCCGCUGCCCAAUGCCAUCCUG |
| NO 100 | | NO 133 | |
| SEQ ID | CCCCAGUUGCAUUCAAUGUUCUGAC | SEQ ID | UUGCCGCUGCCCAAUGCCAUCCUGG |
| NO 101 | | NO 134 | |
| SEQ ID | CCCAGUUGCAUUCAAUGUUCUGACA | SEQ ID | UGCCGCUGCCCAAUGCCAUCCUGGA |
| NO 102 | | NO 135 | |
| SEQ ID | CCAGUUGCAUUCAAUGUUCUGACAA | SEQ ID | GCCGCUGCCCAAUGCCAUCCUGGAG |
| NO 103 | | NO 136 | |
| SEQ ID | CAGUUGCAUUCAAUGUUCUGACAAC | SEQ ID | CCGCUGCCCAAUGCCAUCCUGGAGU |

| | |
|---|---|
| NO 104 | NO 137 |

| | | | |
|---|---|---|---|
| SEQ ID | AGUUGCAUUCAAUGUUCUGACAACA | SEQ ID | CGCUGCCCAAUGCCAUCCUGGAGUU |
| NO 105 | | NO 138 | |
| SEQ ID | UCC UGU AGA AUA CUG GCA UC | SEQ ID | UGUUUUUGAGGAUUGCUGAA |
| NO 106 | | NO 139 | |
| SEQ ID | UGCAGACCUCCUGCCACCGCAGAUU | SEQ ID | UGUUCUGACAACAGUUUGCCGCUGCC |
| NO 107 | CA | NO 140 | CAAUGCCAUCCUGG |
| SEQ ID | UUGCAGACCUCCUGCCACCGCAGAU | SEQ ID | UUUGCCICUGCCCAAUGCCAUCCUG |
| NO 108 | UCAGGCUUC | NO 557 | |
| | | PS305 | |
| SEQ ID | UUUGCCGCUICCCAAUGCCAUCCUG | SEQ ID | UUUGCCGCUGCCCAIUGCCAUCCUG |
| NO 558 | | NO 566 | |
| SEQ ID | UUUGCCGCUGCCCAAUICCAUCCUG | SEQ ID | UUUGCCGCUGCCCAAUGCCIUCCUG |
| NO 559 | | NO 567 | |
| SEQ ID | UUUICCGCUGCCCAAUGCCAUCCUG | SEQ ID | UUUICCICUGCCCAAUGCCAUCCUG |
| NO 560 | | NO 568 | |
| SEQ ID | UUUGCCGCUGCCCAAUGCCAUCCUI | SEQ ID | UUUGCCGCUGCCCAAIGCCAUCCUG |
| NO 561 | | NO 569 | |
| SEQ ID | IUUGCCGCUGCCCAAUGCCAUCCUG | SEQ ID | UUUGCCGCUGCCCAAUGCCAICCUG |
| NO 562 | | NO 570 | |
| SEQ ID | UIUGCCGCUGCCCAAUGCCAUCCUG | SEQ ID | UUUGCCGCUGCCCAAUGCCAUCCIG |
| NO 563 | | NO 571 | |
| SEQ ID | UUIGCCGCUGCCCAAUGCCAUCCUG | SEQ ID | UUUGCCGCUGCCCIAUGCCAUCCUG |
| NO 564 | | NO 572 | |
| SEQ ID | UUUGCCGCIGCCCAAUGCCAUCCUG | | |
| NO 565 | | | |

DMD Gene Exon 53

| | | | |
|---|---|---|---|
| SEQ ID | CUCUGGCCUGUCCUAAGACCUGCUC | SEQ ID | CAGCUUCUUCCUUAGCUUCCAGCCA |
| NO 141 | | NO 165 | |
| SEQ ID | UCUGGCCUGUCCUAAGACCUGCUCA | SEQ ID | AGCUUCUUCCUUAGCUUCCAGCCAU |
| NO 142 | | NO 166 | |
| SEQ ID | CUGGCCUGUCCUAAGACCUGCUCAG | SEQ ID | GCUUCUUCCUUAGCUUCCAGCCAUU |
| NO 143 | | NO 167 | |
| SEQ ID | UGGCCUGUCCUAAGACCUGCUCAGC | SEQ ID | CUUCUUCCUUAGCUUCCAGCCAUUG |
| NO 144 | | NO 168 | |
| SEQ ID | GGCCUGUCCUAAGACCUGCUCAGCU | SEQ ID | UUCUUCCUUAGCUUCCAGCCAUUGU |
| NO 145 | | NO 169 | |
| SEQ ID | GCCUGUCCUAAGACCUGCUCAGCUU | SEQ ID | UCUUCCUUAGCUUCCAGCCAUUGUG |
| NO 146 | | NO 170 | |
| SEQ ID | CCUGUCCUAAGACCUGCUCAGCUUC | SEQ ID | CUUCCUUAGCUUCCAGCCAUUGUGU |
| NO 147 | | NO 171 | |
| SEQ ID | CUGUCCUAAGACCUGCUCAGCUUCU | SEQ ID | UUCCUUAGCUUCCAGCCAUUGUGUU |
| NO 148 | | NO 172 | |
| SEQ ID | UGUCCUAAGACCUGCUCAGCUUCUU | SEQ ID | UCCUUAGCUUCCAGCCAUUGUGUUG |
| NO 149 | | NO 173 | |
| SEQ ID | GUCCUAAGACCUGCUCAGCUUCUUC | SEQ ID | CCUUAGCUUCCAGCCAUUGUGUUGA |
| NO 150 | | NO 174 | |
| SEQ ID | UCCUAAGACCUGCUCAGCUUCUUCC | SEQ ID | CUUAGCUUCCAGCCAUUGUGUUGAA |
| NO 151 | | NO 175 | |
| SEQ ID | CCUAAGACCUGCUCAGCUUCUUCCU | SEQ ID | UUAGCUUCCAGCCAUUGUGUUGAAU |
| NO 152 | | NO 176 | |
| SEQ ID | CUAAGACCUGCUCAGCUUCUUCCUU | SEQ ID | UAGCUUCCAGCCAUUGUGUUGAAUC |
| NO 153 | | NO 177 | |
| SEQ ID | UAAGACCUGCUCAGCUUCUUCCUUA | SEQ ID | AGCUUCCAGCCAUUGUGUUGAAUCC |
| NO 154 | | NO 178 | |
| SEQ ID | AAGACCUGCUCAGCUUCUUCCUUAG | SEQ ID | GCUUCCAGCCAUUGUGUUGAAUCCU |
| NO 155 | | NO 179 | |
| SEQ ID | AGACCUGCUCAGCUUCUUCCUUAGC | SEQ ID | CUUCCAGCCAUUGUGUUGAAUCCUU |
| NO 156 | | NO 180 | |
| SEQ ID | GACCUGCUCAGCUUCUUCCUUAGCU | SEQ ID | UUCCAGCCAUUGUGUUGAAUCCUUU |
| NO 157 | | NO 181 | |
| SEQ ID | ACCUGCUCAGCUUCUUCCUUAGCUU | SEQ ID | UCCAGCCAUUGUGUUGAAUCCUUUA |
| NO 158 | | NO 182 | |
| SEQ ID | CCUGCUCAGCUUCUUCCUUAGCUUC | SEQ ID | CCAGCCAUUGUGUUGAAUCCUUUAA |
| NO 159 | | NO 183 | |
| SEQ ID | CUGCUCAGCUUCUUCCUUAGCUUCC | SEQ ID | CAGCCAUUGUGUUGAAUCCUUUAAC |
| NO 160 | | NO 184 | |
| SEQ ID | UGCUCAGCUUCUUCCUUAGCUUCCA | SEQ ID | AGCCAUUGUGUUGAAUCCUUUAACA |
| NO 161 | | NO 185 | |
| SEQ ID | GCUCAGCUUCUUCCUUAGCUUCCAG | SEQ ID | GCCAUUGUGUUGAAUCCUUUAACAU |
| NO 162 | | NO 186 | |
| SEQ ID | CUCAGCUUCUUCCUUAGCUUCCAGC | SEQ ID | CCAUUGUGUUGAAUCCUUUAACAUU |
| NO 163 | | NO 187 | |
| SEQ ID | UCAGCUUCUUCCUUAGCUUCCAGCC | SEQ ID | CAUUGUGUUGAAUCCUUUAACAUUU |
| NO 164 | | NO 188 | |

DMD Gene Exon 44

| | | | |
|---|---|---|---|
| SEQ ID | UCAGCUUCUGUUAGCCACUG | SEQ ID | AGCUUCUGUUAGCCACUGAUUAAA |
| NO 189 | | NO 214 | |
| SEQ ID | UUCAGCUUCUGUUAGCCACU | SEQ ID | CAGCUUCUGUUAGCCACUGAUUAA |
| NO 190 | | NO 215 | A |
| SEQ ID | UUCAGCUUCUGUUAGCCACUG | SEQ ID | AGCUUCUGUUAGCCACUGAUUAAA |
| NO 191 | | NO 216 | |
| SEQ ID | UCAGCUUCUGUUAGCCACUGA | SEQ ID | AGCUUCUGUUAGCCACUGAU |
| NO 192 | | NO 217 | |
| SEQ ID | UUCAGCUUCUGUUAGCCACUGA | SEQ ID | GCUUCUGUUAGCCACUGAUU |
| NO 193 | | NO 218 | |
| SEQ ID | UCAGCUUCUGUUAGCCACUGA | SEQ ID | AGCUUCUGUUAGCCACUGAUU |
| NO 194 | | NO 219 | |
| SEQ ID | UUCAGCUUCUGUUAGCCACUGA | SEQ ID | GCUUCUGUUAGCCACUGAUUA |
| NO 195 | | NO 220 | |
| SEQ ID | UCAGCUUCUGUUAGCCACUGAU | SEQ ID | AGCUUCUGUUAGCCACUGAUUA |
| NO 196 | | NO 221 | |
| SEQ ID | UUCAGCUUCUGUUAGCCACUGAU | SEQ ID | GCUUCUGUUAGCCACUGAUUAA |
| NO 197 | | NO 222 | |
| SEQ ID | UCAGCUUCUGUUAGCCACUGAUU | SEQ ID | AGCUUCUGUUAGCCACUGAUUAA |
| NO 198 | | NO 223 | |
| SEQ ID | UUCAGCUUCUGUUAGCCACUGAUU | SEQ ID | GCUUCUGUUAGCCACUGAUUAAA |
| NO 199 | | NO 224 | |
| SEQ ID | UCAGCUUCUGUUAGCCACUGAUUA | SEQ ID | AGCUUCUGUUAGCCACUGAUUAAA |
| NO 200 | | NO 225 | |
| SEQ ID | UUCAGCUUCUGUUAGCCACUGAUA | SEQ ID | GCUUCUGUUAGCCACUGAUUAAA |
| NO 201 | | NO 226 | |
| SEQ ID | UCAGCUUCUGUUAGCCACUGAUUAA | SEQ ID | CCAUUUGUAUUUAGCAUGUUCCC |
| NO 202 | | NO 227 | |
| SEQ ID | UUCAGCUUCUGUUAGCCACUGAUUAA | SEQ ID | AGAUACCAUUUGUAUUUAGC |
| NO 203 | | NO 228 | |
| SEQ ID | UCAGCUUCUGUUAGCCACUGAUUAAA | SEQ ID | GCCAUUUCUCAACAGAUCU |
| NO 204 | | NO 229 | |
| SEQ ID | UUCAGCUUCUGUUAGCCACUGAUUAAA | SEQ ID | GCCAUUUCUCAACAGAUCUGUCA |
| NO 205 | | NO 230 | |
| SEQ ID | CAGCUUCUGUUAGCCACUG | SEQ ID | AUUCUCAGGAAUUUGUGUCUUUC |
| NO 206 | | NO 231 | |
| SEQ ID | CAGCUUCUGUUAGCCACUGAU | SEQ ID | UCUCAGGAAUUUGUGUCUUUC |
| NO 207 | | NO 232 | |
| SEQ ID | AGCUUCUGUUAGCCACUGAUU | SEQ ID | GUUCAGCUUCUGUUAGCC |
| NO 208 | | NO 233 | |
| SEQ ID | CAGCUUCUGUUAGCCACUGAUU | SEQ ID | CUGAUUAAAUAUCUUUAUAU C |
| NO 209 | | NO 234 | |
| SEQ ID | AGCUUCUGUUAGCCACUGAUUA | SEQ ID | GCCGCCAUUUCUCAACAG |
| NO 210 | | NO 235 | |
| SEQ ID | CAGCUUCUGUUAGCCACUGAUUA | SEQ ID | GUAUUUAGCAUGUUCCCA |
| NO 211 | | NO 236 | |
| SEQ ID | AGCUUCUGUUAGCCACUGAUUAA | SEQ ID | CAGGAAUUUGUGUCUUUC |
| NO 212 | | NO 237 | |
| SEQ ID | CAGCUUCUGUUAGCCACUGAUUAA | SEQ ID | UCAICUUCUGUUAGCCACUG |
| NO 213 | | NO 575 | |
| SEQ ID | UCAGCUUCUIUUAGCCACUG | SEQ ID | UCAGCUUCUGUUAGCCACUI |
| NO 573 | | NO 576 | |
| SEQ ID | UCAGCUUCUGUUAICCACUG | | |
| NO 574 | | | |

DMD Gene Exon 46

| | | | |
|---|---|---|---|
| SEQ ID | GCUUUUCUUUUAGUUGCUGCUCUUU | SEQ ID | CCAGGUUCAAGUGGGAUACUAGCAA |
| NO 238 | | NO 265 | |
| SEQ ID | CUUUUCUUUUAGUUGCUGCUCUUUU | SEQ ID | CAGGUUCAAGUGGGAUACUAGCAAU |
| NO 239 | | NO 266 | |
| SEQ ID | UUUUCUUUUAGUUGCUGCUCUUUUC | SEQ ID | AGGUUCAAGUGGGAUACUAGCAAUG |
| NO 240 | | NO 267 | |
| SEQ ID | UUUCUUUUAGUUGCUGCUCUUUUCC | SEQ ID | GGUUCAAGUGGGAUACUAGCAAUGU |
| NO 241 | | NO 268 | |
| SEQ ID | UUCUUUUAGUUGCUGCUCUUUUCCA | SEQ ID | GUUCAAGUGGGAUACUAGCAAUGUU |
| NO 242 | | NO 269 | |
| SEQ ID | UCUUUUAGUUGCUGCUCUUUUCCAG | SEQ ID | UUCAAGUGGGAUACUAGCAAUGUUA |
| NO 243 | | NO 270 | |
| SEQ ID | CUUUUAGUUGCUGCUCUUUUCCAGG | SEQ ID | UCAAGUGGGAUACUAGCAAUGUUAU |
| NO 244 | | NO 271 | |
| SEQ ID | UUUUAGUUGCUGCUCUUUUCCAGGU | SEQ ID | CAAGUGGGAUACUAGCAAUGUUAUC |
| NO 245 | | NO 272 | |
| SEQ ID | UUUAGUUGCUGCUCUUUUCCAGGUU | SEQ ID | AAGUGGGAUACUAGCAAUGUUAUCU |
| NO 246 | | NO 273 | |
| SEQ ID | UUAGUUGCUGCUCUUUUCCAGGUUC | SEQ ID | AGUGGGAUACUAGCAAUGUUAUCUG |
| NO 247 | | NO 274 | |
| SEQ ID | UAGUUGCUGCUCUUUUCCAGGUUCA | SEQ ID | GUGGGAUACUAGCAAUGUUAUCUGC |
| NO 248 | | NO 275 | |
| SEQ ID | AGUUGCUGCUCUUUUCCAGGUUCAA | SEQ ID | UGGGAUACUAGCAAUGUUAUCUGCU |
| NO 249 | | NO 276 | |
| SEQ ID | GUUGCUGCUCUUUUCCAGGUUCAAG | SEQ ID | GGGAUACUAGCAAUGUUAUCUGCUU |
| NO 250 | | NO 277 | |
| SEQ ID | UUGCUGCUCUUUUCCAGGUUCAAGU | SEQ ID | GGAUACUAGCAAUGUUAUCUGCUUC |
| NO 251 | | NO 278 | |
| SEQ ID | UCCUGCUCUUUUCCAGGUUCAAGUG | SEQ ID | GAUACUAGCAAUGUUAUCUGCUUCC |
| NO 252 | | NO 279 | |
| SEQ ID | GCUGCUCUUUUCCAGGUUCAAGUGG | SEQ ID | AUACUAGCAAUGUUAUCUGCUUCCU |
| NO 253 | | NO 280 | |
| SEQ ID | CUGCUCUUUUCCAGGUUCAAGUGGG | SEQ ID | UACUAGCAAUGUUAUCUGCUUCCUC |
| NO 254 | | NO 281 | |
| SEQ ID | UGCUCUUUUCCAGGUUCAAGUGGGA | SEQ ID | ACUAGCAAUGUUAUCUGCUUCCUCC |
| NO 255 | | NO 282 | |
| SEQ ID | GCUCUUUUCCAGGUUCAAGUGGGAC | SEQ ID | CUAGCAAUGUUAUCUGCUUCCUCCA |
| NO 256 | | NO 283 | |
| SEQ ID | CUCUUUUCCAGGUUCAAGUGGGAUA | SEQ ID | UAGCAAUGUUAUCUGCUUCCUCCAA |
| NO 257 | | NO 284 | |
| SEQ ID | UCUUUUCCAGGUUCAAGUGGGAUAC | SEQ ID | AGCAAUGUUAUCUGCUUCCUCCAAC |
| NO 258 | | NO 285 | |
| SEQ ID | UCUUUUCCAGGUUCAAGUGG | SEQ ID | GCAAUGUUAUCUGCUUCCUCCAACC |
| NO 259 | | NO 286 | |
| SEQ ID | CUUUUCCAGGUUCAAGUGGGAUACU | SEQ ID | CAAUGUUAUCUGCUUCCUCCAACCA |
| NO 260 | | NO 287 | |
| SEQ ID | UUUUCCAGGUUCAAGUGGGAUACUA | SEQ ID | AAUGUUAUCUGCUUCCUCCAACCAU |
| NO 261 | | NO 288 | |
| SEQ ID | UUUCCAGGUUCAAGUGGGAUACUAG | SEQ ID | AUGUUAUCUGCUUCCUCCAACCAUA |
| NO 262 | | NO 289 | |
| SEQ ID | UUCCAGGUUCAAGUGGGAUACUAGC | SEQ ID | UGUUAUCUGCUUCCUCCAACCAUAA |
| NO 263 | | NO 290 | |
| SEQ ID | UCCAGGUUCAAGUGGGAUACUAGCA | | |
| NO 264 | | | |

DMD Gene Exon 52

| | | | |
|---|---|---|---|
| SEQ ID | AGCCUCUUGAUUGCUGGUCUUGUUU | SEQ ID | UUGGGCAGCGGUAAUGAGUUCUUCC |
| NO 291 | | NO 326 | |
| SEQ ID | GCCUCUUGAUUGCUGGUCUUGUUUU | SEQ ID | UGGGCAGCGGUAAUGAGUUCUUCCA |
| NO 292 | | NO 327 | |
| SEQ ID | CCUCUUGAUUGCUGGUCUUGUUUUU | SEQ ID | GGGCAGCGGUAAUGAGUUCUUCCAA |
| NO 293 | | NO 328 | |
| SEQ ID | CCUCUUGAUUGCUGGUCUUG | SEQ ID | GGCAGCGGUAAUGAGUUCUUCCAAC |
| NO 294 | | NO 329 | |
| SEQ ID | CUCUUGAUUGCUGGUCUUGUUUUUC | SEQ ID | GCAGCGGUAAUGAGUUCUUCCAACU |
| NO 295 | | NO 330 | |
| SEQ ID | UCUUGAUUGCUGGUCUUGUUUUUCA | SEQ ID | CAGCGGUAAUGAGUUCUUCCAACUG |
| NO 296 | | NO 331 | |
| SEQ ID | CUUGAUUGCUGGUCUUGUUUUUCAA | SEQ ID | AGCGGUAAUGAGUUCUUCCAACUGG |
| NO 297 | | NO 332 | |
| SEQ ID | UUGAUUGCUGGUCUUGUUUUUCAAA | SEQ ID | GCGGUAAUGAGUUCUUCCAACUGGG |
| NO 298 | | NO 333 | |
| SEQ ID | UGAUUGCUGGUCUUGUUUUUCAAAU | SEQ ID | CGGUAAUGAGUUCUUCCAACUGGGG |
| NO 299 | | NO 334 | |
| SEQ ID | GAUUGCUGGUCUUGUUUUUCAAAUU | SEQ ID | GGUAAUGAGUUCUUCCAACUGGGGA |
| NO 300 | | NO 335 | |
| SEQ ID | GAUUGCUGGUCUUGUUUUUC | SEQ ID | GGUAAUGAGUUCUUCCAACUGG |
| NO 301 | | NO 336 | |
| SEQ ID | AUUGCUGGUCUUGUUUUUCAAAUUU | SEQ ID | GUAAUGAGUUCUUCCAACUGGGGAC |
| NO 302 | | NO 337 | |
| SEQ ID | UUGCUGGUCUUGUUUUUCAAAUUUU | SEQ ID | UAAUGAGUUCUUCCAACUGGGGACG |
| NO 303 | | NO 338 | |
| SEQ ID | UGCUGGUCUUGUUUUUCAAAUUUUG | SEQ ID | AAUGAGUUCUUCCAACUGGGGACGC |
| NO 304 | | NO 339 | |
| SEQ ID | GCUGGUCUUGUUUUUCAAAUUUUGG | SEQ ID | AUGAGUUCUUCCAACUGGGGACGCC |
| NO 305 | | NO 340 | |
| SEQ ID | CUGGUCUUGUUUUUCAAAUUUUGGG | SEQ ID | UGAGUUCUUCCAACUGGGGACGCCU |
| NO 306 | | NO 341 | |
| SEQ ID | UGGUCUUGUUUUUCAAAUUUUGGGC | SEQ ID | GAGUUCUUCCAACUGGGGACGCCUC |
| NO 307 | | NO 342 | |
| SEQ ID | GGUCUUGUUUUUCAAAUUUUGGGCA | SEQ ID | AGUUCUUCCAACUGGGGACGCCUCU |
| NO 308 | | NO 343 | |
| SEQ ID | GUCUUGUUUUUCAAAUUUUGGGCAG | SEQ ID | GUUCUUCCAACUGGGGACGCCUCUG |
| NO 309 | | NO 344 | |
| SEQ ID | UCUUGUUUUUCAAAUUUUGGGCAGC | SEQ ID | UUCUUCCAACUGGGGACGCCUCUGU |
| NO 310 | | NO 345 | |
| SEQ ID | CUUGUUUUUCAAAUUUUGGGCAGCG | SEQ ID | UCUUCCAACUGGGGACGCCUCUGUU |
| NO 311 | | NO 346 | |
| SEQ ID | UUGUUUUUCAAAUUUUGGGCAGCGG | SEQ ID | CUUCCAACUGGGGACGCCUCUGUUC |
| NO 312 | | NO 347 | |
| SEQ ID | UGUUUUUCAAAUUUUGGGCAGCGGU | SEQ ID | UUCCAACUGGGGACGCCUCUGUUCC |
| NO 313 | | NO 348 | |
| SEQ ID | GUUUUUCAAAUUUUGGGCAGCGGUA | SEQ ID | UCCAACUGGGGACGCCUCUGUUCCA |
| NO 314 | | NO 349 | |
| SEQ ID | UUUUUCAAAUUUUGGGCAGCGGUAA | SEQ ID | CCAACUGGGGACGCCUCUGUUCCAA |
| NO 315 | | NO 350 | |
| SEQ ID | UUUUCAAAUUUUGGGCAGCGGUAAU | SEQ ID | CAACUGGGGACGCCUCUGUUCCAAA |
| NO 316 | | NO 351 | |
| SEQ ID | UUUCAAAUUUUGGGCAGCGGUAAUG | SEQ ID | AACUGGGGACGCCUCUGUUCCAAAU |
| NO 317 | | NO 352 | |
| SEQ ID | UUCAAAUUUUGGGCAGCGGUAAUGA | SEQ ID | ACUGGGGACGCCUCUGUUCCAAAUC |
| NO 318 | | NO 353 | |
| SEQ ID | UCAAAUUUUGGGCAGCGGUAAUGAG | SEQ ID | CUGGGGACGCCUCUGUUCCAAAUCC |
| NO 319 | | NO 354 | |
| SEQ ID | CAAAUUUUGGGCAGCGGUAAUGAGU | SEQ ID | UGGGGACGCCUCUGUUCCAAAUCCU |
| NO 320 | | NO 355 | |
| SEQ ID | AAAUUUUGGGCAGCGGUAAUGAGUU | SEQ ID | GGGGACGCCUCUGUUCCAAAUCCUG |
| NO 321 | | NO 356 | |
| SEQ ID | AAUUUUGGGCAGCGGUAAUGAGUUC | SEQ ID | GGGACGCCUCUGUUCCAAAUCCUGC |
| NO 322 | | NO 357 | |
| SEQ ID | AUUUUGGGCAGCGGUAAUGAGUUCU | SEQ ID | GGACGCCUCUGUUCCAAAUCCUGCA |
| NO 323 | | NO 358 | |
| SEQ ID | UUUUGGGCAGCGGUAAUGAGUUCUU | SEQ ID | GACGCCUCUGUUCCAAAUCCUGCAU |
| NO 324 | | NO 359 | |
| SEQ ID | UUUGGGCAGCGGUAAUGAGUUCUUC | | |
| NO 325 | | | |

DMD Gene Exon 50

| | | | |
|---|---|---|---|
| SEQ ID | CCAAUAGUGGUCAGUCCAGGAGCUA | SEQ ID | CUAGGUCAGGCUGCUUUGCCCUCAG |
| NO 360 | | NO 386 | |
| SEQ ID | CAAUAGUGGUCAGUCCAGGAGCUAG | SEQ ID | UAGGUCAGGCUGCUUUGCCCUCAGC |
| NO 361 | | NO 387 | |
| SEQ ID | AAUAGUGGUCAGUCCAGGAGCUAGG | SEQ ID | AGGUCAGGCUGCUUUGCCCUCAGCU |
| NO 362 | | NO 388 | |
| SEQ ID | AUAGUGGUCAGUCCAGGAGCUAGGU | SEQ ID | GGUCAGGCUGCUUUGCCCUCAGCUC |
| NO 363 | | NO 389 | |
| SEQ ID | AUAGUGGUCAGUCCAGGAGCU | SEQ ID | GUCAGGCUGCUUUGCCCUCAGCUCU |
| NO 364 | | NO 390 | |
| SEQ ID | UAGUGGUCAGUCCAGGAGCUAGGUC | SEQ ID | UCAGGCUGCUUUGCCCUCAGCUCUU |
| NO 365 | | NO 391 | |
| SEQ ID | AGUGGUCAGUCCAGGAGCUAGGUCA | SEQ ID | CAGGCUGCUUUGCCCUCAGCUCUUG |
| NO 366 | | NO 392 | |
| SEQ ID | GUGGUCAGUCCAGGAGCUAGGUCAG | SEQ ID | AGGCUGCUUUGCCCUCAGCUCUUGA |
| NO 367 | | NO 393 | |
| SEQ ID | UGGUCAGUCCAGGAGCUAGGUCAGG | SEQ ID | GGCUGCUUUGCCCUCAGCUCUUGAA |
| NO 368 | | NO 394 | |
| SEQ ID | GGUCAGUCCAGGAGCUAGGUCAGGC | SEQ ID | GCUGCUUUGCCCUCAGCUCUUGAAG |
| NO 369 | | NO 395 | |
| SEQ ID | GUCAGUCCAGGAGCUAGGUCAGGCU | SEQ ID | CUGCUUUGCCCUCAGCUCUUGAAGU |
| NO 370 | | NO 396 | |
| SEQ ID | UCAGUCCAGGAGCUAGGUCAGGCUG | SEQ ID | UGCUUUGCCCUCAGCUCUUGAAGUA |
| NO 371 | | NO 397 | |
| SEQ ID | CAGUCCAGGAGCUAGGUCAGGCUGC | SEQ ID | GCUUUGCCCUCAGCUCUUGAAGUAA |
| NO 372 | | NO 398 | |
| SEQ ID | AGUCCAGGAGCUAGGUCAGGCUGCU | SEQ ID | CUUUGCCCUCAGCUCUUGAAGUAAA |
| NO 373 | | NO 399 | |
| SEQ ID | GUCCAGGAGCUAGGUCAGGCUGCUU | SEQ ID | UUUGCCCUCAGCUCUUGAAGUAAAC |
| NO 374 | | NO 400 | |
| SEQ ID | UCCAGGAGCUAGGUCAGGCUGCUUU | SEQ ID | UUGCCCUCAGCUCUUGAAGUAAACG |
| NO 375 | | NO 401 | |
| SEQ ID | CCAGGAGCUAGGUCAGGCUGCUUUG | SEQ ID | UGCCCUCAGCUCUUGAAGUAAACGG |
| NO 376 | | NO 402 | |
| SEQ ID | CAGGAGCUAGGUCAGGCUGCUUUGC | SEQ ID | GCCCUCAGCUCUUGAAGUAAACGGU |
| NO 377 | | NO 403 | |
| SEQ ID | AGGAGCUAGGUCAGGCUGCUUUGCC | SEQ ID | CCCUCAGCUCUUGAAGUAAACGGUU |
| NO 378 | | NO 404 | |
| SEQ ID | GGAGCUAGGUCAGGCUGCUUUGCCC | SEQ ID | CCUCAGCUCUUGAAGUAAAC |
| NO 379 | | NO 405 | |
| SEQ ID | GAGCUAGGUCAGGCUGCUUUGCCCU | SEQ ID | CCUCAGCUCUUGAAGUAAACG |
| NO 380 | | NO 406 | |
| SEQ ID | AGCUAGGUCAGGCUGCUUUGCCCUC | SEQ ID | CUCAGCUCUUGAAGUAAACG |
| NO 381 | | NO 407 | |
| SEQ ID | GCUAGGUCAGGCUGCUUUGCCCUCA | SEQ ID | CCUCAGCUCUUGAAGUAAACGGUUU |
| NO 382 | | NO 408 | |
| SEQ ID | CUCAGCUCUUGAAGUAAACGGUUUA | SEQ ID | UCAGCUCUUGAAGUAAACGGUUUAC |
| NO 383 | | NO 409 | |
| SEQ ID | CAGCUCUUGAAGUAAACGGUUUACC | SEQ ID | AGCUCUUGAAGUAAACGGUUUACCG |
| NO 384 | | NO 410 | |
| SEQ ID | GCUCUUGAAGUAAACGGUUUACCGC | SEQ ID | CUCUUGAAGUAAACGGUUUACCGCC |
| NO 385 | | NO 411 | |

DMD Gene Exon 43

| | | | |
|---|---|---|---|
| SEQ ID | CCACAGGCGUUGCACUUUGCAAUGC | SEQ ID | UCUUCUUGCUAUGAAUAAUGUCAAU |
| NO 412 | | NO 443 | |
| SEQ ID | CACAGGCGUUGCACUUUGCAAUGCU | SEQ ID | CUUCUUGCUAUGAAUAAUGUCAAUC |
| NO 413 | | NO 444 | |
| SEQ ID | ACAGGCGUUGCACUUUGCAAUGCUG | SEQ ID | UUCUUGCUAUGAAUAAUGUCAAUCC |
| NO 414 | | NO 445 | |
| SEQ ID | CAGGCGUUGCACUUUGCAAUGCUGC | SEQ ID | UCUUGCUAUGAAUAAUGUCAAUCCG |
| NO 415 | | NO 446 | |
| SEQ ID | AGGCGUUGCACUUUGCAAUGCUGCU | SEQ ID | CUUGCUAUGAAUAAUGUCAAUCCGA |
| NO 416 | | NO 447 | |
| SEQ ID | GGCGUUGCACUUUGCAAUGCUGCUG | SEQ ID | UUGCUAUGAAUAAUGUCAAUCCGAC |
| NO 417 | | NO 448 | |
| SEQ ID | GCGUUGCACUUUGCAAUGCUGCUGU | SEQ ID | UGCUAUGAAUAAUGUCAAUCCGACC |
| NO 418 | | NO 449 | |
| SEQ ID | CGUUGCACUUUGCAAUGCUGCUGUC | SEQ ID | GCUAUGAAUAAUGUCAAUCCGACCU |
| NO 419 | | NO 450 | |
| SEQ ID | CGUUGCACUUUGCAAUGCUGCUG | SEQ ID | CUAUGAAUAAUGUCAAUCCGACCUG |
| NO 420 | | NO 451 | |
| SEQ ID | GUUGCACUUUGCAAUGCUGCUGUCU | SEQ ID | UAUGAAUAAUGUCAAUCCGACCUGA |
| NO 421 | | NO 452 | |
| SEQ ID | UUGCACUUUGCAAUGCUGCUGUCUU | SEQ ID | AUGAAUAAUGUCAAUCCGACCUGAG |
| NO 422 | | NO 453 | |
| SEQ ID | UGCACUUUGCAAUGCUGCUGUCUUC | SEQ ID | UGAAUAAUGUCAAUCCGACCUGAGC |
| NO 423 | | NO 454 | |
| SEQ ID | GCACUUUGCAAUGCUGCUGUCUUCU | SEQ ID | GAAUAAUGUCAAUCCGACCUGAGCU |
| NO 424 | | NO 455 | |
| SEQ ID | CACUUUGCAAUGCUGCUGUCUUCUU | SEQ ID | AAUAAUGUCAAUCCGACCUGAGCUU |
| NO 425 | | NO 456 | |
| SEQ ID | ACUUUGCAAUGCUGCUGUCUUCUUG | SEQ ID | AUAAUGUCAAUCCGACCUGAGCUUU |
| NO 426 | | NO 457 | |
| SEQ ID | CUUUGCAAUGCUGCUGUCUUCUUGC | SEQ ID | UAAUGUCAAUCCGACCUGAGCUUUG |
| NO 427 | | NO 458 | |
| SEQ ID | UUUGCAAUGCUGCUGUCUUCUUGCU | SEQ ID | AAUGUCAAUCCGACCUGAGCUUUGU |
| NO 428 | | NO 459 | |
| SEQ ID | UUGCAAUGCUGCUGUCUUCUUGCUA | SEQ ID | AUGUCAAUCCGACCUGAGCUUUGUU |
| NO 429 | | NO 460 | |
| SEQ ID | UGCAAUGCUGCUGUCUUCUUGCUAU | SEQ ID | UGUCAAUCCGACCUGAGCUUUGUUG |
| NO 430 | | NO 461 | |
| SEQ ID | GCAAUGCUGCUGUCUUCUUGCUAUG | SEQ ID | GUCAAUCCGACCUGAGCUUUGUUGU |
| NO 431 | | NO 462 | |
| SEQ ID | CAAUGCUGCUGUCUUCUUGCUAUGA | SEQ ID | UCAAUCCGACCUGAGCUUUGUUGUA |
| NO 432 | | NO 463 | |
| SEQ ID | AAUGCUGCUGUCUUCUUGCUAUGAA | SEQ ID | CAAUCCGACCUGAGCUUUGUUGUAG |
| NO 433 | | NO 464 | |
| SEQ ID | AUGCUGCUGUCUUCUUGCUAUGAAU | SEQ ID | AAUCCGACCUGAGCUUUGUUGUAGA |
| NO 434 | | NO 465 | |
| SEQ ID | UGCUGCUGUCUUCUUGCUAUGAAUA | SEQ ID | AUCCGACCUGAGCUUUGUUGUAGAC |
| NO 435 | | NO 466 | |
| SEQ ID | GCUGCUGUCUUCUUGCUAUGAAUAA | SEQ ID | UCCGACCUGAGCUUUGUUGUAGACU |
| NO 436 | | NO 467 | |
| SEQ ID | CUGCUGUCUUCUUGCUAUGAAUAAU | SEQ ID | CCGACCUGAGCUUUGUUGUAGACUA |
| NO 437 | | NO 468 | |
| SEQ ID | UGCUGUCUUCUUGCUAUGAAUAAUG | SEQ ID | CGACCUGAGCUUUGUUGUAG |
| NO 438 | | NO 469 | |
| SEQ ID | GCUGUCUUCUUGCUAUGAAUAAUGU | SEQ ID | CGACCUGAGCUUUGUUGUAGACUAU |
| NO 439 | | NO 470 | |
| SEQ ID | CUGUCUUCUUGCUAUGAAUAAUGUC | SEQ ID | GACCUGAGCUUUGUUGUAGACUAUC |
| NO 440 | | NO 471 | |
| SEQ ID | UGUCUUCUUGCUAUGAAUAAUGUCA | SEQ ID | ACCUGAGCUUUGUUGUAGACUAUCA |
| NO 441 | | NO 472 | |
| SEQ ID | GUCUUCUUGCUAUGAAUAAUGUCAA | SEQ ID | CCUGAGCUUUGUUGUAGACUAUC |
| NO 442 | | NO 473 | |

DMD Gene Exon 6

| | | | |
|---|---|---|---|
| SEQ ID | CAUUUUUGACCUACAUGUGG | SEQ ID | AUUUUUGACCUACAUGGGAAA G |
| NO 474 | | NO 479 | |
| SEQ ID | UUUGACCUACAUGUGGAAAG | SEQ ID | UACGAGUUGAUUGUCGGACCCAG |
| NO 475 | | NO 480 | |
| SEQ ID | UACAUUUUUGACCUACAUGUGGAAA | SEQ ID | GUGGUCUCCUUACCUAUGACUGUGG |
| NO 476 | G | NO 481 | |
| SEQ ID | GGUCUCCUUACCUAUGA | SEQ ID | UGUCUCAGUAAUCUUCUUACCUAU |
| NO 477 | | NO 482 | |
| SEQ ID | UCUUACCUAUGACUAUGGAUGAGA | | |
| NO 478 | | | |

DMD Gene Exon 7

| | | | |
|---|---|---|---|
| SEQ ID | UGCAUGUUCCAGUCGUUGUGUGG | SEQ ID | AUUUACCAACCUUCAGGAUCGAGUA |
| NO 483 | | NO 485 | |
| SEQ ID | CACUAUUCCAGUCAAAUAGGUCUGG | SEQ ID | GGCCUAAAACACAUACACAUA |
| NO 484 | | NO 486 | |

DMD Gene Exon 8

| | | | |
|---|---|---|---|
| SEQ ID | GAUAGGUGGUAUCAACAUCUGUAA | SEQ ID | UGUUGUUGUUUAUGCUCAUU |
| NO 487 | | NO 490 | |
| SEQ ID | GAUAGGUGGUAUCAACAUCUG | SEQ ID | GUACAUUAAGAUGGACUUC |
| NO 488 | | NO 491 | |
| SEQ ID | CUUCCUGGAUGGCUUGAAU | | |
| NO 489 | | | |

DMD Gene Exon 55

| | | | |
|---|---|---|---|
| SEQ ID | CUGUUGCAGUAAUCUAUGAG | SEQ ID | UGCCAUUGUUUCAUCAGCUCUUU |
| NO 492 | | NO 495 | |
| SEQ ID | UGCAGUAAUCUAUGAGUUUC | SEQ ID | UCCUGUAGGACAUUGGCAGU |
| NO 493 | | NO 496 | |
| SEQ ID | GAGUCUUCUAGGAGCCUU | SEQ ID | CUUGGAGUCUUCUAGGAGCC |
| NO 494 | | NO 497 | |

DMD Gene Exon 2

| | | | |
|---|---|---|---|
| SEQ ID | CCAUUUUGUGAAUGUUUUCUUUUG | SEQ ID | GAAAAUUGUGCAUUUACCCAUUUU |
| NO 498 | AACAUC | NO 500 | |
| SEQ ID | CCCAUUUUGUGAAUGUUUUCUUUU | SEQ ID | UUGUGCAUUUACCCAUUUUGUG |
| NO 499 | | NO 501 | |

DMD Gene Exon 11

| | | | |
|---|---|---|---|
| SEQ ID | CCCUGAGGCAUUCCCAUCUUGAAU | SEQ ID | CUUGAAUUUAGGAGAUUCAUCUG |
| NO 502 | | NO 504 | |
| SEQ ID | AGGACUUACUUGCUUUGUUU | SEQ ID | CAUCUUCUGAUAAUUUUCCUGUU |
| NO 503 | | NO 505 | |

DMD Gene Exon 17

| | | | |
|---|---|---|---|
| SEQ ID | CCAUUACAGUUGUCUGUGUU | SEQ ID | UAAUCUGCCUCUUCUUUUGG |
| NO 506 | | NO 508 | |
| SEQ ID | UGACAGCCUGUGAAAUCUGUGAG | | |
| NO 507 | | | |

DMD Gene Exon 19

| | | | |
|---|---|---|---|
| SEQ ID | CAGCAGUAGUUGUCAUCUGC | SEQ ID | GCCUGAGCUGAUCUGCUGGCAUCUUG |
| NO 509 | | NO 511 | CAGUU |
| SEQ ID | GCCUGAGCUGAUCUGCUGGCAUCUU | SEQ ID | UCUGCUGGCAUCUUGC |
| NO 510 | GC | NO 512 | |

DMD Gene Exon 21

| | | | |
|---|---|---|---|
| SEQ ID | GCCGGUUGACUUCAUCCUGUGC | SEQ ID | CUGCAUCCAGGAACAUGGGUCC |
| NO 513 | | NO 516 | |
| SEQ ID | GUCUGCAUCCAGGAACAUGGGUC | SEQ ID | GUUGAAGAUCUGAUAGCCGGUUGA |
| NO 514 | | NO 517 | |
| SEQ ID | UACUUACUGUCUGUAGCUCUUUCU | | |
| NO 515 | | | |

DMD Gene Exon 57

| | | | |
|---|---|---|---|
| SEQ ID | UAGGUGCCUGCCGGCUU | SEQ ID | CUGAACUGCUGGAAAGUCGCC |
| NO 518 | | NO 520 | |
| SEQ ID | UUCAGCUGUAGCCACACC | SEQ ID | CUGGCUUCCAAAUGGGACCUGAAAAA |
| NO 519 | | NO 521 | GAAC |

DMD Gene Exon 59

| | |
|---|---|
| SEQ ID | CAAUUUUUCCCACUCAGUAUU |

| | | | |
|---|---|---|---|
| NO 522 | | SEQ ID | UCCUCAGGAGGCAGCUCUAAAU |
| SEQ ID | UUGAAGUUCCUGGAGUCUU | NO 524 | |
| NO 523 | | | |

DMD Gene Exon 62

| | | | |
|---|---|---|---|
| SEQ ID | UGGCUCUCUCCCAGGG | SEQ ID | GGGCACUUUGUUUGGCG |
| NO 525 | | NO 527 | |
| SEQ ID | GAGAUGGCUCUCUCCCAGGGACCCU | | |
| NO 526 | GG | | |

DMD Gene Exon 63

| | | | |
|---|---|---|---|
| SEQ ID | GGUCCCAGCAAGUUGUUUG | SEQ ID | GUAGAGCUCUGUCAUUUUGGG |
| NO 528 | | NO 530 | |
| SEQ ID | UGGGAUGGUCCCAGCAAGUUGUUUG | | |
| NO 529 | | | |

DMD Gene Exon 65

| | | | |
|---|---|---|---|
| SEQ ID | GCUCAAGAGAUCCACUGCAAAAAAC | SEQ ID | UCUGCAGGAUAUCCAUGGGCUGGUC |
| NO 531 | | NO 533 | |
| SEQ ID | GCCAUACGUACGUAUCAUAAACAUU | | |
| NO 532 | C | | |

DMD Gene Exon 66

| | |
|---|---|
| SEQ ID | GAUCCUCCCUGUUCGUCCCCUAUUA |
| NO 534 | UG |

DMD Gene Exon 69

| | |
|---|---|
| SEQ ID | UGCUUUAGACUCCUGUACCUGAUA |
| NO 535 | |

DMD Gene Exon 75

| | | | |
|---|---|---|---|
| SEQ ID | GGCGGCCUUUGUGUUGAC | SEQ ID | CCUUUAUGUUCGUGCUGCU |
| NO 536 | | NO 538 | |
| SEQ ID | GGACAGGCCUUUAUGUUCGUGCUGC | | |
| NO 537 | | | |

Human IGF-1 Isoform 4 amino acid sequence
SEQ ID NO 577:
MGKISSLPTQLFKCCFCDFLKVKMHTMSSSHLFYLALCLLTFTSSATAGPETLCGAELV
DALQFVCGDRGFYFNKPTGYGSSSRRAPQTGIVDECCFRSCDLRRLEMYCAPLKPAKSA
RSVRAQRHTDMPKTQKEVHLKNASRGSAGNKNYRM

What is claimed is:

1. An isolated antisense oligonucleotide fully complementary to a contiguous sequence of an of exon of dystrophin pre-mRNA, wherein said contiguous sequence is fully complementary to at least 21 nucleotides of a base sequence, said oligonucleotide comprises at least one position wherein a guanosine base is substituted with an inosine base, wherein said oligonucleotide comprises a modification and is capable of inducing skipping of said exon of human dystrophin pre-mRNA.

2. The oligonucleotide of claim 1, said oligonucleotide being 25-50 nucleotides in length.

3. The oligonucleotide of claim 1, said oligonucleotide being 25-30 nucleotides in length.

4. The oligonucleotide of claim 1, said oligonucleotide comprising from one to four inosine bases.

5. The oligonucleotide of claim 1, further comprising a base and/or sugar modification.

6. The oligonucleotide of claim 1, wherein said oligonucleotide is a 2′-O-methyl phosphorothioate oligonucleotide.

7. The oligonucleotide of claim 1, wherein said exon is selected from the group consisting of 51, 45, 53, 44, 46, 52, 50, 43 and 55.

8. The oligonucleotide of claim 1, wherein said base sequence is selected from the group consisting of: SEQ ID NO: 2-473, 539-576.

9. The oligonucleotide of claim 8, further comprising a base and/or sugar modification.

10. The oligonucleotide of claim 1, wherein said oligonucleotide is a 2′-O-methyl phosphorothioate oligonucleotide.

11. The oligonucleotide of claim 1, which is a locked nucleic acid oligonucleotide (LNA).

12. The oligonucleotide of claim 1, which is a peptide nucleic acid oligonucleotide (PNA).

13. The oligonucleotide of claim 1, which is a morpholino phosphorodiamidate oligonucleotide (PMO).

14. The oligonucleotide of claim 1, further comprising modified internucleoside linkage.

15. A pharmaceutical composition comprising the oligonucleotide of claim 1 and a pharmaceutically acceptable carrier.

16. A method for inducing skipping of an exon of human dystrophin pre-mRNA in a muscle cell, the method comprising contacting said cell with an oligonucleotide of claim 1 for a time and under conditions which permit exon skipping.

17. A method for inducing skipping of an exon of human dystrophin pre-mRNA in a human subject, the method comprising administering an oligonucleotide of claim 1 to said subject in an amount and for a time which is effective to induce exon skipping.

18. A method for alleviating one or more symptom(s) of Duchenne Muscular Dystrophy or Becker Muscular Dystrophy in an individual, the method comprising administering to said individual an oligonucleotide of claim 1.

19. A method for inducing and/or promoting skipping of exon 43, 44, 45, 46, 50, 51, 52 or 53 of the dystrophin pre-mRNA in a patient, the method comprising administering an oligonucleotide of claim 3 to said patient.
